SEQUENCE LISTING (101 sequences)

SEQ ID NO: 1 - protein, 92 aa, synthetic construct
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - DNA, 276 bp, synthetic construct
  source 1..276: mol_type = other DNA
gctcctgatgtccaggactgtcctgagtgcaccctgcaggagaaccccttctttagccagcctggtgcccccatcctgcagtgcatgggctgctgcttctccagagcatatcccacacccctgaggagcaagaagaccatgctggtgcagaagaatgtgacgagtgaatccacttgttgcgtagctaaaagctataaccgggtaacagtgatggggggctttaaagttgaaaaccatactgcatgccactgtagcacttgctactaccacaagtct

SEQ ID NO: 3 - DNA, 276 bp, synthetic construct
  source 1..276: mol_type = other DNA
gcacccgatgtccaagactgccccgagtgcacattgcaagaaaatccctttttctctcagccaggggctcctatcttgcagtgtatgggctgttgcttctcaagagcctatcccacaccccttcgatcaaaaaaaacaatgcttgttcaaaaaaatgtgacctctgagtcaacctgctgcgtggctaaatcctacaaccgcgtaacagttatggggggattcaaagtagaaaatcatacagcatgccactgttccacttgctactatcataagagt

SEQ ID NO: 4 - protein, 111 aa, synthetic construct
NSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 5 - DNA, 333 bp, synthetic construct
  source 1..333: mol_type = other DNA
aatagctgtgagctgactaacataaccatagccattgagaaggaagagtgtagattctgcattagcattaacaccacctggtgtgctggctactgctacacccgtgacctagtctataaggaccctgctaggcctaagatccagaagacctgcacctttaaagaactagtgtatgagacagtgagggtgcctggctgtgcccaccatgctgatagcctgtacacatacccagtagccacccagtgtcactgtggtaaatgtgatagtgatagcactgactgcactgtgagaggactaggccctagctactgtagctttggtgagatgaaggag

SEQ ID NO: 6 - protein, 223 aa, synthetic construct
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 7 - DNA, 669 bp, synthetic construct
  source 1..669: mol_type = other DNA
caggtgcagcttgtccagtccggaggcggccctgtgaagcctggggggtcattgagattgtcctgtgcagcatccggatttatgttcagggcttactccatgaactgggtgcgccaggctccagggaaggggttggaatgggtgtccagcatcagcagctctggaaggtacatccactacgccgactccgtgaagggcaggttcaccatttcccgggacaacgctaagaacagcctgtacctccagatgaactccctccgggctgaggacaccgccgtgtactactgcgcccgggagacagtgatggccgggaaagccctggattactggggccaaggcactctggtcacagtcagctcagcctctacaaaaggccctagtgtgtttcctctggccccatcaagtaagagcacctcagaggggaccgccgccctgggctgcctggtcaaagattactttccagagcccgtgacagtgagttggaacagtggggctctgacaagcggggtccatacatttcccgccgtgctgcagagcagcgggctgtatagcctgagcagcgtcgtcactgtgcccagctcttctctcggcacacagacttacatttgcaatgtgaaccacaagcccagcaacactaaggtcgataagaaggtggagcccaagtcctcc

SEQ ID NO: 8 - DNA, 669 bp, synthetic construct
  source 1..669: mol_type = other DNA
caagtgcaactggttcaatctggcggcggccccgtaaagccaggtggtagcctgcgtctgagttgtgccgctagtgggttcatgttccgagcctactctatgaattgggtgaggcaggccccagggaaaggtttggagtgggtttcctcaatcagctcaagtggtcgatatattcactatgctgactcagtcaagggtcgtttcaccatcagtcgggataatgccaagaacagcttgtatcttcaaatgaacagcttgagagctgaagataccgctgtgtactattgtgctagggaaactgtcatggctggcaaggccttggactactgggggcagggtactcttgtaactgttagtagcgcaagcactaaaggcccatcagtttttccattggcaccatcatccaaatctacatctgagggcactgcagcattggggtgtctcgtcaaagattattttcccgagccagtcacagtatcctggaatagcggggctttgacatctggggtgcatacatttcctgctgttttgcaatccagcggactctactctctgagttctgttgtaacagtccctagcagttctctgggaacccaaacttacatttgtaacgtgaatcacaagccttccaacacaaaggtcgataaaaaggtcgagcctaaatcatcc

SEQ ID NO: 9 - DNA, 333 bp, synthetic construct
  source 1..333: mol_type = other DNA
aattcctgtgaactgacaaacataacaatcgcaattgaaaaggaagagtgcaggttctgtatctctataaacaccacctggtgtgctgggtactgctacacccgcgacctggtttataaagatcccgcacggcccaaaatacagaaaacctgcacttttaaagagttggtttacgaaactgtcagagtcccaggctgtgcccatcacgcagatagcctctacacctaccctgttgccacccaatgtcattgcggaaagtgtgattctgatagtactgattgcacagttcgaggcctgggaccatcttattgttcctttggagagatgaaggag

SEQ ID NO: 10 - DNA, 60 bp, synthetic construct
  source 1..60: mol_type = other DNA
atggagacccacagccaggtgttcgtgtacatgctgctgtggctgagcggggtggagggg

SEQ ID NO: 11 - DNA, 669 bp, synthetic construct
  misc_feature 1..669: note = SL335H
  source 1..669: mol_type = other DNA
caagtgcaattggtgcagtcaggtggcggcccagtaaaacccggtgggtctctgagattgagctgcgctgcatccggatttatgttccgtgcctacagcatgaattgggtgcggcaagctcctggaaagggactcgaatgggtttccagtattagcagttctgggaggtatatacattatgctgactcagtgaaagggaggttcacaattagccgggacaatgccaaaaactctctctatctgcagatgaacagccttcgcgccgaagataccgccgtttattactgcgctcgggagactgtgatggccggtaaggctcttgactattggggacagggaactttggtgactgtttcatctgcctctacaaagggacccagcgtttttccattggcacctagtagcaagtccacatctgaaggtacagctgctttggggtgtttggtgaaagactacttccccgaaccagtgacagtttcatggaactccggcgctttgactagtggagtgcataccttcccagctgttcttcagagtagtgggctttatagtttgagtagcgtcgtgactgtcccatcatcctctctcggcacacaaacctatatctgcaatgtaaaccataagccatcaaacaccaaagtagacaagaaagttgagccaaagtccagc

SEQ ID NO: 12 - DNA, 669 bp, synthetic construct
  misc_feature 1..669: note = SL335H
  source 1..669: mol_type = other DNA
caagtccagcttgtgcagtccggcggtggaccagtgaagcctgggggatcactgcgcctctcctgtgccgcttcggggttcatgttccgggcatactcgatgaactgggttagacaggctcccggaaagggcctggaatgggtgtccagtatctcaagctcgggccgctacattcattatgcggacagcgtgaagggcagattcaccattagccgggacaatgccaagaactccctgtacttgcaaatgaactccctgagggccgaggatactgcggtgtactattgtgctcgggagactgtgatggccggaaaggccctggactactggggccagggcacccttgtgaccgtgtcctccgcctcgaccaagggaccgtcggtgtttcctctggcgccgtcctcgaagtcaacctccgagggaaccgccgccctgggttgcctcgtgaaagactacttccctgaacccgtgactgtgtcctggaacagcggtgccctgacctccggggtgcatactttccctgcggtgctgcagtcgtccggactctactccctgtcatccgtcgtgaccgtgccttcctcctccctgggaacccagacctacatctgcaacgtgaaccacaagccctccaacaccaaggtcgacaagaaagtcgagcccaagtccagc

SEQ ID NO: 13 - protein, 215 aa, synthetic construct
  REGION 1..215: note = SL335L
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 14 - DNA, 645 bp, synthetic construct
  misc_feature 1..645: note = SL335L
  source 1..645: mol_type = other DNA
gacatcgtcctgacccagagccccgggacactgagcctgtcccccggggagacagccacactgagctgcagggccagccagtccgtcggctccaacctggcctggtaccagcagaagcccgggcaggcccccaggctgctgatttacggcgccagcacaggcgccacaggggtgcccgccaggtttagcggcagccggagcggcacagattttacactgacaatcacatccctgcagcccgaggattttgccacatactactgccagcagtactactccttcctggccaagacattcggccagggcacccagctggagatcaagcggaccgtggccgcccccagcgtgtttattttcccccccagcgatgagcagctgaagtccggcaccgcctccgtcgtgtgcctgctgaacaacttctacccccgggaggccaaggtccagtggaaggtcgataacgccctgcagagcgggaactcccaggagtccgtgaccgagcaggactccaaggacagcacctactccctgtccaacaccctgaccctgagcaaggccgactacgagaagcacaaggtctacgcctgcgaggtgacccaccagggcctgtcctcccccgtgaccaagagctttaaccggggggagtcc

SEQ ID NO: 15 - DNA, 645 bp, synthetic construct
  misc_feature 1..645: note = SL335L
  source 1..645: mol_type = other DNA
gatatcgtgctgactcagtcgcctggtaccctttccctgtcacccggagaaactgccaccctgagctgtagagcgagccagtccgtgggctccaacctggcctggtatcaacagaagcctgggcaggcccctcgcctgttgatctacggcgccagcactggtgccaccggcgtgccagctcggttctccggctcccggtcggggactgacttcaccctcaccattacgagcctgcagcccgaagatttcgcgacctactactgccaacagtactactcattcctggctaagactttcggacaaggcacccagctcgagatcaagcggaccgtggcagcaccgtccgtgttcattttcccgccgtccgacgagcaacttaagtccggaaccgcctctgtcgtgtgcctcctcaacaacttctacccgcgcgaggccaaggtccagtggaaggtcgacaacgcgctgcagtccggaaattcacaggaaagcgtgaccgaacaggactccaaggattcgacctactccctgtccaacactctgaccctgtcgaaagccgactacgagaagcacaaagtgtacgcctgcgaagtgacacatcagggactcagctcgcccgtgaccaagagcttcaacaggggagagtcg

SEQ ID NO: 16 - protein, 8 aa, synthetic construct
  REGION 1..8: note = Linker 1
GSAPAPGS

SEQ ID NO: 17 - DNA, 24 bp, synthetic construct
  misc_feature 1..24: note = Linker 1
  source 1..24: mol_type = other DNA
gggtcagcccctgccccaggtagc

SEQ ID NO: 18 - DNA, 24 bp, synthetic construct
  misc_feature 1..24: note = Linker 1
  source 1..24: mol_type = other DNA
ggctcggccccggcaccgggcagc

SEQ ID NO: 19 - protein, 322 aa, synthetic construct
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSSDVPSGPGAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 20 - DNA, 966 bp, synthetic construct
  source 1..966: mol_type = other DNA
caggtgcagcttgtccagtccggaggcggccctgtgaagcctggggggtcattgagattgtcctgtgcagcatccggatttatgttcagggcttactccatgaactgggtgcgccaggctccagggaaggggttggaatgggtgtccagcatcagcagctctggaaggtacatccactacgccgactccgtgaagggcaggttcaccatttcccgggacaacgctaagaacagcctgtacctccagatgaactccctccgggctgaggacaccgccgtgtactactgcgcccgggagacagtgatggccgggaaagccctggattactggggccaaggcactctggtcacagtcagctcagcctctacaaaaggccctagtgtgtttcctctggccccatcaagtaagagcacctcagaggggaccgccgccctgggctgcctggtcaaagattactttccagagcccgtgacagtgagttggaacagtggggctctgacaagcggggtccatacatttcccgccgtgctgcagagcagcgggctgtatagcctgagcagcgtcgtcactgtgcccagctcttctctcggcacacagacttacatttgcaatgtgaaccacaagcccagcaacactaaggtcgataagaaggtggagcccaagtcctccgatgtgcctagtggccctggagctcctgatgtccaggactgtcctgagtgcaccctgcaggagaaccccttctttagccagcctggtgcccccatcctgcagtgcatgggctgctgcttctccagagcatatcccacacccctgaggagcaagaagaccatgctggtgcagaagaatgtgacgagtgaatccacttgttgcgtagctaaaagctataaccgggtaacagtgatggggggctttaaagttgaaaaccatactgcatgccactgtagcacttgctactaccacaagtct

SEQ ID NO: 21 - protein, 341 aa, synthetic construct
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGESGGGGSGGGGSGGGGSNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 22 - protein, 5 aa, synthetic construct
  REGION 1..5: note = Heavy chain variable domain CDR1
SYGIS

SEQ ID NO: 23 - protein, 17 aa, synthetic construct
  REGION 1..17: note = Heavy chain variable domain CDR2
WINTYSGGTKYAQKFQG

SEQ ID NO: 24 - protein, 15 aa, synthetic construct
  REGION 1..15: note = Heavy chain variable domain CDR3
LGHCQRGICSDALDT

SEQ ID NO: 25 - protein, 17 aa, synthetic construct
  REGION 1..17: note = Heavy chain variable domain CDR2
RINTYNGNTGYAQRLQG

SEQ ID NO: 26 - protein, 5 aa, synthetic construct
  REGION 1..5: note = Heavy chain variable domain CDR1
NYGIH

SEQ ID NO: 27 - protein, 17 aa, synthetic construct
  REGION 1..17: note = Heavy chain variable domain CDR2
SISYDGSNKYYADSVKG

SEQ ID NO: 28 - protein, 16 aa, synthetic construct
  REGION 1..16: note = Heavy chain variable domain CDR3
DVHYYGSGSYYNAFDI

SEQ ID NO: 29 - protein, 5 aa, synthetic construct
  REGION 1..5: note = Heavy chain variable domain CDR1
SYAMS

SEQ ID NO: 30 - protein, 17 aa, synthetic construct
  REGION 1..17: note = Heavy chain variable domain CDR2
VISHDGGFQYYADSVKG

SEQ ID NO: 31 - protein, 11 aa, synthetic construct
  REGION 1..11: note = Heavy chain variable domain CDR3
AGWLRQYGMDV

SEQ ID NO: 32 - protein, 5 aa, synthetic construct
  REGION 1..5: note = Heavy chain variable domain CDR1
AYWIA

SEQ ID NO: 33 - protein, 17 aa, synthetic construct
  REGION 1..17: note = Heavy chain variable domain CDR2
MIWPPDADARYSPSFQG

SEQ ID NO: 34 - protein, 8 aa, synthetic construct
  REGION 1..8: note = Heavy chain variable domain CDR3
LYSGSYSP

SEQ ID NO: 35 - protein, 5 aa, synthetic construct
  REGION 1..5: note = Heavy chain variable domain CDR1
AYSMN

SEQ ID NO: 36 - protein, 17 aa, synthetic construct
  REGION 1..17: note = Heavy chain variable domain CDR2
SISSSGRYIHYADSVKG

SEQ ID NO: 37 - protein, 11 aa, synthetic construct
  REGION 1..11: note = Heavy chain variable domain CDR3
ETVMAGKALDY

SEQ ID NO: 38 - protein, 11 aa, synthetic construct
  REGION 1..11: note = Light chain variable domain CDR1
RASQSISRYLN

SEQ ID NO: 39 - protein, 7 aa, synthetic construct
  REGION 1..7: note = Light chain variable domain CDR2
GASRLES

SEQ ID NO: 40 - protein, 9 aa, synthetic construct
  REGION 1..9: note = Light chain variable domain CDR3
QQSDSVPVT

SEQ ID NO: 41 - protein, 11 aa, synthetic construct
  REGION 1..11: note = Light chain variable domain CDR1
RASQSISSYLN

SEQ ID NO: 42 - protein, 7 aa, synthetic construct
  REGION 1..7: note = Light chain variable domain CDR2
AASSLQS

SEQ ID NO: 43 - protein, 10 aa, synthetic construct
  REGION 1..10: note = Light chain variable domain CDR3
QQSYSTPPYT

SEQ ID NO: 44 - protein, 11 aa, synthetic construct
  REGION 1..11: note = Light chain variable domain CDR1
RASQSIFNYVA

SEQ ID NO: 45 - protein, 7 aa, synthetic construct
  REGION 1..7: note = Light chain variable domain CDR2
DASNRAT

SEQ ID NO: 46 - protein, 11 aa, synthetic construct
  REGION 1..11: note = Light chain variable domain CDR3
QQRSKWPPTWT

SEQ ID NO: 47 - protein, 12 aa, synthetic construct
  REGION 1..12: note = Light chain variable domain CDR1
RASETVSSRQLA

SEQ ID NO: 48 - protein, 7 aa, synthetic construct
  REGION 1..7: note = Light chain variable domain CDR2
GASSRAT

SEQ ID NO: 49 - protein, 9 aa, synthetic construct
  REGION 1..9: note = Light chain variable domain CDR3
QQYGSSPRT

SEQ ID NO: 50 - protein, 12 aa, synthetic construct
  REGION 1..12: note = Light chain variable domain CDR1
RASQSVSSSSLA

SEQ ID NO: 51 - protein, 9 aa, synthetic construct
  REGION 1..9: note = Light chain variable domain CDR3
QKYSSYPLT

SEQ ID NO: 52 - protein, 11 aa, synthetic construct
  REGION 1..11: note = Light chain variable domain CDR1
RASQSVGSNLA

SEQ ID NO: 53 - protein, 7 aa, synthetic construct
  REGION 1..7: note = Light chain variable domain CDR2
GASTGAT

SEQ ID NO: 54 - protein, 10 aa, synthetic construct
  REGION 1..10: note = Light chain variable domain CDR3
QQYYSFLAKT

SEQ ID NO: 55 - protein, 124 aa, synthetic construct
  REGION 1..124: note = Heavy chain variable domain 1
QVQLLQSGAEVKKPGASVKVSCKASGYTFTSYGISWVRQAPGQGLEWVGWINTYSGGTKYAQKFQGRVTMTRDTSISTVYMELSGLKSDDTAVYYCARLGHCQRGICSDALDTWGQGTLVTVSS

SEQ ID NO: 56 - protein, 124 aa, synthetic construct
  REGION 1..124: note = Heavy chain variable domain 2
EVQLLQSGAEVKEPGASVKVSCKASGYTFSSYGISWVRQAPGQGLEWVGRINTYNGNTGYAQRLQGRVTMTTDTSTSIAYMEVRSLRSDDTAVYYCARLGHCQRGICSDALDTWGQGTMVTVSS

SEQ ID NO: 57 - protein, 125 aa, synthetic construct
  REGION 1..125: note = Heavy chain variable domain 3
QVQLVQSGGGVVQTGGSLRLSCAASGFTFRNYGIHWVRQAPGKGLEWVASISYDGSNKYYADSVKGRFTISRDNSRNTVHVQMDSLRGGDTAVYYCARDVHYYGSGSYYNAFDIWGQGTLVTVSS

SEQ ID NO: 58 - protein, 120 aa, synthetic construct
  REGION 1..120: note = Heavy chain variable domain 4
QVQLVQSGGGLVQPGGSLRLSCAASGFTFSSYAMSWVRQAPGKGLEWLSVISHDGGFQYYADSVKGRFTVSRDNSKNTLYLQMNSLRAEDTAVYYCARAGWLRQYGMDVWGQGTLVTVSS

SEQ ID NO: 59 - protein, 117 aa, synthetic construct
  REGION 1..117: note = Heavy chain variable domain 5
EVQLVQSGTEVKKPGESLKISCKISGYSFTAYWIAWVRQMPGKGLEWMGMIWPPDADARYSPSFQGQVTFSVDKSISTAYLQWHSLKTSDTAVYYCARLYSGSYSPWGQGTLVTVSS

SEQ ID NO: 60 - protein, 120 aa, synthetic construct
  REGION 1..120: note = Heavy chain variable domain 6
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSS

SEQ ID NO: 61 - protein, 108 aa, synthetic construct
  REGION 1..108: note = Light chain variable domain 1
ELVLTQSPSSLSASVGDRVTITCRASQSISRYLNWYQQKPGKAPKLLIYGASRLESGVPSRFSGSGSGTDFTLTISSLQPEDFATYYCQQSDSVPVTFGQGTRLEIKR

SEQ ID NO: 62 - protein, 109 aa, synthetic construct
  REGION 1..109: note = Light chain variable domain 2
DIVLTQSPSSLSASVGDRVTITCRASQSISSYLNWYQQKPGKAPKLLIYAASSLQSGVPSRFSGSGSGTDFTLTISSLQPEDFATYYCQQSYSTPPYTFGQGTKLEIKR

SEQ ID NO: 63 - protein, 110 aa, synthetic construct
  REGION 1..110: note = Light chain variable domain 3
ELVLTQSPGTLSLSPGERATLSCRASQSIFNYVAWYQQKPGQAPRLLIYDASNRATGIPARFSGSGSGTDFTLTISSLEPEDFAVYYCQQRSKWPPTWTFGQGTRVDIKR

SEQ ID NO: 64 - protein, 109 aa, synthetic construct
  REGION 1..109: note = Light chain variable domain 4
ELVLTQSPGTLSLSPGERATLSCRASETVSSRQLAWYQQKPGQAPRLLIYGASSRATGIPDRFSGSGSGTDFTLTISRLEPEDSAVFYCQQYGSSPRTFGGGTKLEIKR

SEQ ID NO: 65 - protein, 109 aa, synthetic construct
  REGION 1..109: note = Light chain variable domain 5
ELVLTQSPGTLSLSPGERATLSCRASQSVSSSSLAWYQQKPGQAPRLLIYGASSRATGIPDRFSGSGSGTDFTLTISSLQPEDAATYYCQKYSSYPLTFGQGTKLEIKR

SEQ ID NO: 66 - protein, 109 aa, synthetic construct
  REGION 1..109: note = Light chain variable domain 6
ELVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 67 - protein, 109 aa, synthetic construct
  REGION 1..109: note = Light chain variable domain 7
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 68 - protein, 103 aa, synthetic construct
  REGION 1..103: note = Heavy chain constant domain
ASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 69 - protein, 107 aa, synthetic construct
  REGION 1..107: note = Light chain constant domain
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 70 - protein, 15 aa, synthetic construct
  REGION 1..15: note = Linker 2
GSAGSAPAPAGSGEF

SEQ ID NO: 71 - protein, 10 aa, synthetic construct
  REGION 1..10: note = Linker 3
GGGGSGGGGS

SEQ ID NO: 72 - protein, 5 aa, synthetic construct
  REGION 1..5: note = Linker 4
GGGGS

SEQ ID NO: 73 - protein, 5 aa, synthetic construct
  REGION 1..5: note = Linker 5
SGGGG

SEQ ID NO: 74 - protein, 5 aa, synthetic construct
  REGION 1..5: note = Linker 6
SRSSG

SEQ ID NO: 75 - protein, 5 aa, synthetic construct
  REGION 1..5: note = Linker 7
SGSSC

SEQ ID NO: 76 - protein, 12 aa, synthetic construct
  REGION 1..12: note = Linker 8
GKSSGSGSESKS

SEQ ID NO: 77 - protein, 6 aa, synthetic construct
  REGION 1..6: note = Linker 9
RPPPPC

SEQ ID NO: 78 - protein, 7 aa, synthetic construct
  REGION 1..7: note = Linker 10
SSPPPPC

SEQ ID NO: 79 - protein, 14 aa, synthetic construct
  REGION 1..14: note = Linker 11
GSTSGSGKSSEGKG

SEQ ID NO: 80 - protein, 18 aa, synthetic construct
  REGION 1..18: note = Linker 12
GSTSGSGKSSEGSGSTKG

SEQ ID NO: 81 - protein, 18 aa, synthetic construct
  REGION 1..18: note = Linker 13
GSTSGSGKPGSGEGSTKG

SEQ ID NO: 82 - protein, 14 aa, synthetic construct
  REGION 1..14: note = Linker 14
EGKSSGSGSESKEF

SEQ ID NO: 83 - protein, 15 aa, synthetic construct
  REGION 1..15: note = Linker 15
GSAGSAPAPAGSGEF

SEQ ID NO: 84 - protein, 10 aa, synthetic construct
  REGION 1..10: note = Linker 16
GGGGSGGGGS

SEQ ID NO: 85 - protein, 15 aa, synthetic construct
GGGGSGGGGSGGGGS

SEQ ID NO: 86 - DNA, 45 bp, synthetic construct
  source 1..45: mol_type = other DNA
ggcggtggaggtagtggaggaggcggtagtggtggcggtggtagt

SEQ ID NO: 87 - DNA, 45 bp, synthetic construct
  source 1..45: mol_type = other DNA
ggcggtggcggctcaggcggaggaggctccgggggaggtgggtcc

SEQ ID NO: 88 - protein, 7 aa, synthetic construct
DVPSGPG

SEQ ID NO: 89 - RNA, 21 nt, synthetic construct
  source 1..21: mol_type = other RNA
gatgtgcctagtggccctgga

SEQ ID NO: 90 - DNA, 21 bp, synthetic construct
  source 1..21: mol_type = other DNA
gacgttccatctggccctggt

SEQ ID NO: 91 - protein, 106 aa, synthetic construct
TVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 92 - DNA, 645 bp, synthetic construct; identical to SEQ ID NO: 14
  source 1..645: mol_type = other DNA

SEQ ID NO: 93 - DNA, 645 bp, synthetic construct
  source 1..645: mol_type = other DNA
gacatagttctcacccaaagtcccgggactctttcattgtcacccggagaaaccgcaacattgtcatgccgagcttcccaatcagtaggtagtaatttggcctggtatcaacaaaaaccaggccaggcaccccgccttctgatatatggagcctctaccggagccacaggtgtaccagcacgttttagcggttctcgttccgggacagactttactttgaccattacctcactgcagcctgaggacttcgctacatattattgccaacaatattattcttttctggccaaaacctttgggcaagggacccagttggagattaagcgaactgttgcagctcccagtgtttttatcttccctcccagtgatgaacaactgaaaagcggtaccgcctcagtggtttgtctccttaataatttctacccacgcgaagccaaggtgcaatggaaggttgataatgctttgcaatcagggaatagtcaggaatctgttacagaacaagattccaaggattcaacatatagcctgtctaataccctcactctgtccaaggctgattacgaaaagcacaaagtctatgcctgtgaggttactcaccaagggctgtcctcccctgttacaaaatctttcaatcggggagagagt

SEQ ID NO: 94 - DNA, 1023 bp, synthetic construct
  source 1..1023: mol_type = other DNA
gacatcgtcctgacccagagccccgggacactgagcctgtcccccggggagacagccacactgagctgcagggccagccagtccgtcggctccaacctggcctggtaccagcagaagcccgggcaggcccccaggctgctgatttacggcgccagcacaggcgccacaggggtgcccgccaggtttagcggcagccggagcggcacagattttacactgacaatcacatccctgcagcccgaggattttgccacatactactgccagcagtactactccttcctggccaagacattcggccagggcacccagctggagatcaagcggaccgtggccgcccccagcgtgtttattttcccccccagcgatgagcagctgaagtccggcaccgcctccgtcgtgtgcctgctgaacaacttctacccccgggaggccaaggtccagtggaaggtcgataacgccctgcagagcgggaactcccaggagtccgtgaccgagcaggactccaaggacagcacctactccctgtccaacaccctgaccctgagcaaggccgactacgagaagcacaaggtctacgcctgcgaggtgacccaccagggcctgtcctcccccgtgaccaagagctttaaccggggggagtccggcggtggaggtagtggaggaggcggtagtggtggcggtggtagtaatagctgtgagctgactaacataaccatagccattgagaaggaagagtgtagattctgcattagcattaacaccacctggtgtgctggctactgctacacccgtgacctagtctataaggaccctgctaggcctaagatccagaagacctgcacctttaaagaactagtgtatgagacagtgagggtgcctggctgtgcccaccatgctgatagcctgtacacatacccagtagccacccagtgtcactgtggtaaatgtgatagtgatagcactgactgcactgtgagaggactaggccctagctactgtagctttggtgagatgaaggag

SEQ ID NO: 95 - protein, 349 aa, synthetic construct
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 96 - DNA, 1047 bp, synthetic construct
  source 1..1047: mol_type = other DNA
caagtgcaactggttcaatctggcggcggccccgtaaagccaggtggtagcctgcgtctgagttgtgccgctagtgggttcatgttccgagcctactctatgaattgggtgaggcaggccccagggaaaggtttggagtgggtttcctcaatcagctcaagtggtcgatatattcactatgctgactcagtcaagggtcgtttcaccatcagtcgggataatgccaagaacagcttgtatcttcaaatgaacagcttgagagctgaagataccgctgtgtactattgtgctagggaaactgtcatggctggcaaggccttggactactgggggcagggtactcttgtaactgttagtagcgcaagcactaaaggcccatcagtttttccattggcaccatcatccaaatctacatctgagggcactgcagcattggggtgtctcgtcaaagattattttcccgagccagtcacagtatcctggaatagcggggctttgacatctggggtgcatacatttcctgctgttttgcaatccagcggactctactctctgagttctgttgtaacagtccctagcagttctctgggaacccaaacttacatttgtaacgtgaatcacaagccttccaacacaaaggtcgataaaaaggtcgagcctaaatcatccggcggtggcggctcaggcggaggaggctccgggggaggtgggtccaattcctgtgaactgacaaacataacaatcgcaattgaaaaggaagagtgcaggttctgtatctctataaacaccacctggtgtgctgggtactgctacacccgcgacctggtttataaagatcccgcacggcccaaaatacagaaaacctgcacttttaaagagttggtttacgaaactgtcagagtcccaggctgtgcccatcacgcagatagcctctacacctaccctgttgccacccaatgtcattgcggaaagtgtgattctgatagtactgattgcacagttcgaggcctgggaccatcttattgttcctttggagagatgaaggag

SEQ ID NO: 97 - protein, 314 aa, synthetic construct
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGESDVPSGPGAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 98 - DNA, 942 bp, synthetic construct
  source 1..942: mol_type = other DNA
gacatagttctcacccaaagtcccgggactctttcattgtcacccggagaaaccgcaacattgtcatgccgagcttcccaatcagtaggtagtaatttggcctggtatcaacaaaaaccaggccaggcaccccgccttctgatatatggagcctctaccggagccacaggtgtaccagcacgttttagcggttctcgttccgggacagactttactttgaccattacctcactgcagcctgaggacttcgctacatattattgccaacaatattattcttttctggccaaaacctttgggcaagggacccagttggagattaagcgaactgttgcagctcccagtgtttttatcttccctcccagtgatgaacaactgaaaagcggtaccgcctcagtggtttgtctccttaataatttctacccacgcgaagccaaggtgcaatggaaggttgataatgctttgcaatcagggaatagtcaggaatctgttacagaacaagattccaaggattcaacatatagcctgtctaataccctcactctgtccaaggctgattacgaaaagcacaaagtctatgcctgtgaggttactcaccaagggctgtcctcccctgttacaaaatctttcaatcggggagagagtgacgttccatctggccctggtgcacccgatgtccaagactgccccgagtgcacattgcaagaaaatccctttttctctcagccaggggctcctatcttgcagtgtatgggctgttgcttctcaagagcctatcccacaccccttcgatcaaaaaaaacaatgcttgttcaaaaaaatgtgacctctgagtcaacctgctgcgtggctaaatcctacaaccgcgtaacagttatggggggattcaaagtagaaaatcatacagcatgccactgttccacttgctactatcataagagt

SEQ ID NO: 99 - protein, 19 aa, synthetic construct
MEWSWVFLFFLSVTTGVLS

SEQ ID NO: 100 - DNA, 57 bp, synthetic construct
  source 1..57: mol_type = other DNA
atggagtggtcctgggtcttcctcttcttcctcagcgtgaccaccggagtgctttcc

SEQ ID NO: 101 - protein, 20 aa, unidentified
METHSQVFVYMLLWLSGVEG

(19) United States
(12) Patent Application Publication
(10) Pub. No.: US 2026/0125443 A1
(43) Pub. Date: May 7, 2026

(54) FUSION PROTEIN COMPRISING FOLLICLE STIMULATING HORMONE AND ANTIGEN-BINDING FRAGMENT TO SERUM ALBUMIN AND COMPOSITIONS AND USES THEREOF

(71) Applicant: Aprilbio Co., Ltd., Chuncheon-si, Gangwon-do (KR)

(72) Inventor: Sang Hoon CHA, Chuncheon-si, Gangwon-do (KR)

(21) Appl. No.: 19/118,074

(22) PCT Filed: Oct. 4, 2023
(86) PCT No.: PCT/IB2023/059986

§ 371 Date: Apr. 3, 2025

(30) Foreign Application Priority Data
Oct. 4, 2022 (KR) 10-2022-0126516

(51) Int. Cl. C07K 14/59; A61K 38/00; A61K 47/68; A61P 5/26; A61P 15/08; C07K 16/18

(52) U.S. Cl. CPC C07K 14/59; A61K 47/6811; A61K 47/6843; A61P 5/26; A61P 15/08; C07K 16/18; A61K 38/00; C07K 2317/55; C07K 2317/92; C07K 2317/94; C07K 2319/30; C07K 2319/31

(57) ABSTRACT

The present application relates to a fusion protein including a follicle stimulating hormone and an antigen-binding fragment to serum albumin and a use thereof. Accordingly, a recombinant fusion protein, which includes an antigen-binding fragment that binds to serum albumin and a human follicle stimulating hormone linked to the antigen-binding fragment, and a long-acting human follicle stimulating hormone formulation including the recombinant fusion protein as an active ingredient are provided.

### CROSS-REFERENCE TO RELATED APPLICATIONS

This application claims priority under 35 U.S.C. § 119 to Korean Appl. No. 10-2022-0126516, filed Oct. 4, 2022, the disclosure of which is incorporated by reference herein in its entirety.

### REFERENCE TO SEQUENCE LISTING SUBMITTED ELECTRONICALLY

This application contains a Sequence Listing which has been submitted in XML format via EFS-Web and is hereby incorporated by reference in its entirety. Said XML copy, created on Oct. 4, 2023, is named 2662-0007WO01_SEQL_ST26 and is 130,077 bytes in size.

### BACKGROUND

### 1. Field

The disclosure relates to a fusion protein including a follicle stimulating hormone and an antigen-binding fragment to serum albumin and uses thereof.

### 2. Description of the Related Art

Infertility refers to a condition in which pregnancy does not happen even after being in a normal conjugal relationship for more than one year. The causes of infertility are estimated to lie in the male in 40%, the female in 40%, both the male and female in 10%, and unknown causes in 10%. In the case of female infertility, main causes include ovulation disorders, hormonal abnormalities, uterine abnormalities, and the like. In the case of male infertility, main causes include hormonal abnormalities, testicular abnormalities due to toxic substances or trauma, sperm abnormalities, and the like. Assisted reproductive technologies in the female infertility treatment include in vitro fertilization-embryo transfer (IVF-ET), gamete intrafallopian transfer (GIFT), zygote intrafallopian transfer (ZIFT), intracytoplasmic sperm injection (ICSI), and the like, such assisted reproductive technologies essentially include ovulation induction processes using follicle stimulating hormone. In addition, follicle stimulating hormone is used for therapeutic purposes in women suffering from anovulation. Furthermore, follicle stimulating hormone is also used for the treatment of male infertility caused by sperm abnormalities.

Meanwhile, human follicle stimulating hormone (hFSH) is glycoprotein hormone produced by the pituitary gland and secreted in the endocrine system and has a dimer structure including an alpha subunit and a beta subunit. The alpha subunit belongs to a glycoprotein composed of 92 amino acids common to the glycoprotein families, such as luteinizing hormone (LH) as a pituitary glycoprotein, human chorionic gonadotropin (hCG) as a placental glycoprotein, and thyroid stimulating hormone (TSH), and has two N-glycosylation sites at positions 52 and 78 of asparagine. The beta subunit is a glycoprotein composed of 111 amino acids specific to hFSH and has two N-glycosylation sites at positions 7 and 24 of asparagine. The hFSH is a glycoprotein in which sugar chains account for about 40% of the total molecular weight, and such glycosylation is known to play an important role in the receptor binding ability and signal transduction activity of hFSH.

The hFSH is known to play an important role in the differentiation and growth of male and female reproductive cells, and thus is used to mature multiple follicles in women who have undergone assisted reproductive technology for the infertility treatment or has been used for the treatment of anovulation. The hFSH is also used for sperm production and sperm count increase in men. Urine-derived hFSH purified from female urine has low efficiency and supply instability, and poses many risks, especially in terms of safety. In this regard, genetically engineered formulations prepared by using genetic engineering technology are being in common use, but due to a short in vivo half-life, there is a limitation that frequent administration of the genetically engineered formulations is required.

Under such a technical background, there is a need to develop a long-acting hormone formulation as a technique for improving therapeutic efficacy of hFSH and increasing convenience of patients.

Antigen-binding fragments that bind to serum albumin are provided in U.S. Pat. Nos. 9,879,077 and 11,773,176, each incorporated herein by reference in its entirety.

### SUMMARY

Provided is a recombinant fusion protein including an antigen-binding fragment that binds to serum albumin and a human follicle stimulating hormone (hFSH).

Provided are a nucleic acid encoding the recombinant fusion protein, an expression vector including the nucleic acid, and a cell transformed with the expression vector.

Provided are a long-acting hFSH formulation or a pharmaceutical composition for infertility treatment, each including the recombinant fusion protein as an active ingredient.

Additional aspects will be set forth in part in the description which follows and, in part, will be apparent from the description, or can be learned by practice of the presented embodiments of the disclosure.

Disclosed herein are recombinant fusion proteins comprising (a) a human follicle stimulating hormone (hFSH) comprising an hFSH alpha subunit and an hFSH beta subunit and (b) an antigen binding fragment (Fab) that binds to serum albumin. In some embodiments, the hFSH alpha subunit and the hFSH beta subunit are linked to terminal regions of the Fab. In some embodiments, the hFSH alpha subunit and the hFSH beta subunit are independently linked to a terminus of the heavy chain constant 1 domain and a terminus of the light chain constant domain of the Fab. In some embodiments, the hFSH alpha subunit and the hFSH beta subunit are independently linked by a linker to a terminus of a heavy chain constant 1 domain and a terminus of a light chain constant domain of the Fab. In other embodiments, the hFSH alpha subunit is linked to a terminus of the light chain constant domain and the hFSH beta subunit is linked to a terminus of the heavy chain constant 1 domain of the Fab.

In some embodiments, the linker comprises 1 to 50 amino acids. In some embodiments, the linker comprises an amino acid sequence of any one of SEQ ID NOS:16, 70-85, and 88.

In some embodiments, the hFSH alpha subunit comprises an amino acid sequence having at least 90% identity to SEQ ID NO:1. In some embodiments, the hFSH alpha subunit comprises an amino acid sequence of SEQ ID NO:1. In some embodiments, the hFSH beta subunit comprises an amino acid sequence having at least 90% identity to SEQ ID NO:4. In some embodiments, the hFSH beta subunit comprises an amino acid sequence of SEQ ID NO:4.

In some embodiments, in the fusion proteins disclosed herein, the heavy chain and the light chain of the Fab are bound by a noncovalent bond.

The Fab can comprise
a heavy chain comprising a heavy chain variable domain comprising
   (a) a heavy chain complementarity determining domain 1 (CDR1) comprising the amino acid sequence of SYGIS (SEQ ID NO:22),
      a heavy chain complementarity determining domain 2 (CDR2) comprising the amino acid sequence of WINTYSGGTKYAQKFQG (SEQ ID NO:23), and
      a heavy chain complementarity determining domain 3 (CDR3) comprising the amino acid sequence of LGHCQRGICSDALDT (SEQ ID NO:24);
   (b) a heavy chain CDR1 comprising the amino acid sequence of SYGIS (SEQ ID NO:22),
      a heavy chain CDR2 comprising the amino acid sequence of RINTYNGNTGYAQRLQG (SEQ ID NO:25), and
      a heavy chain CDR3 comprising the amino acid sequence of LGHCQRGICSDALDT (SEQ ID NO:24);
   (c) a heavy chain CDR1 comprising the amino acid sequence of NYGIH (SEQ ID NO:26),
      a heavy chain CDR2 comprising the amino acid sequence of SISYDGSNKYYADSVKG (SEQ ID NO:27), and
      a heavy chain CDR3 comprising the amino acid sequence of DVHYYGSGSYYNAFDI (SEQ ID NO:28);
   (d) a heavy chain CDR1 comprising the amino acid sequence of SYAMS (SEQ ID NO:29),
      a heavy chain CDR2 comprising the amino acid sequence of VISHDGGFQYYADSVKG (SEQ ID NO:30), and
      a heavy chain CDR3 comprising the amino acid sequence of AGWLRQYGMDV (SEQ ID NO:31);
   (e) a heavy chain CDR1 comprising the amino acid sequence of AYWIA (SEQ ID NO:32),
      a heavy chain CDR2 comprising the amino acid sequence of MIWPPDADARYSPSFQG (SEQ ID NO:33), and
      a heavy chain CDR3 comprising the amino acid sequence of LYSGSYSP (SEQ ID NO:34); or
   (f) a heavy chain CDR1 comprising the amino acid sequence of AYSMN (SEQ ID NO:35),
      a heavy chain CDR2 comprising the amino acid sequence of SISSSGRYIHYADSVKG (SEQ ID NO:36), and
      a heavy chain CDR3 comprising the amino acid sequence of ETVMAGKALDY (SEQ ID NO:37); and a light chain comprising a light chain variable domain comprising
   (g) a light chain CDR1 comprising the amino acid sequence of RASQSISRYLN (SEQ ID NO:38),
      a light chain CDR2 comprising the amino acid sequence of GASRLES (SEQ ID NO:39), and
      a light chain CDR3 comprising the amino acid sequence of QQSDSVPVT (SEQ ID NO:40);
   (h) a light chain CDR1 comprising the amino acid sequence of RASQSISSYLN (SEQ ID NO:41),
      a light chain CDR2 comprising the amino acid sequence of AASSLQS (SEQ ID NO:42), and
      a light chain CDR3 comprising the amino acid sequence of QQSYSTPPYT (SEQ ID NO:43);
   (i) a light chain CDR1 comprising the amino acid sequence of RASQSIFNYVA (SEQ ID NO:44),
      a light chain CDR2 comprising the amino acid sequence of DASNRAT (SEQ ID NO:45), and
      a light chain CDR3 comprising the amino acid sequence of QQRSKWPPTWT (SEQ ID NO:46);
   (j) a light chain CDR1 comprising the amino acid sequence of RASETVSSRQLA (SEQ ID NO:47),
      a light chain CDR2 comprising the amino acid sequence of GASSRAT (SEQ ID NO:48), and
      a light chain CDR3 comprising the amino acid sequence of QQYGSSPRT (SEQ ID NO:49);
   (k) a light chain CDR1 comprising the amino acid sequence of RASQSVSSSSLA (SEQ ID NO:50),
      a light chain CDR2 comprising the amino acid sequence of GASSRAT (SEQ ID NO:48), and
      a light chain CDR3 comprising the amino acid sequence of QKYSSYPLT (SEQ ID NO:51); or
   (l) a light chain CDR1 comprising the amino acid sequence of RASQSVGSNLA (SEQ ID NO:52),
      a light chain CDR2 comprising the amino acid sequence of GASTGAT (SEQ ID NO:53), and
      a light chain CDR3 comprising the amino acid sequence of QQYYSFLAKT (SEQ ID NO:54).

In some embodiments, in the fusion proteins disclosed herein, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37; and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. In some embodiments, the light chain variable domain comprises an amino acid sequence having at least 90% identity to SEQ ID NO:61, 62, 63, 64, 65, 66, or 67. In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence having at least 90% identity to SEQ ID NO:68. In some embodiments, the light chain constant domain comprises an amino acid sequence having at least 90% identity to SEQ ID NO:69 or 91.

In some embodiments, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and wherein the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. In some embodiments, the heavy chain variable domain comprises the amino acid sequence of SEQ ID NO:60.

In some embodiments, the light chain variable domain comprises an amino acid sequence having at least 90% identity to SEQ ID NO:61, 62, 63, 64, 65, 66, or 67. In some embodiments, the light chain variable domain comprises the amino acid sequence of SEQ ID NO:67.

In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence.

In some embodiments, the light chain constant domain comprises an amino acid sequence having at least 90% identity to SEQ ID NO:69 or 91.

In some embodiments, the Fab comprises a heavy chain comprising the amino acid sequence of SEQ ID NO:6 and a light chain comprising the amino acid sequence of SEQ ID NO:14. In some embodiments, the Fab comprises a heavy chain constant 1 domain comprising the amino acid sequence of SEQ ID NO:68 linked to the heavy chain variable domain; and a light chain constant domain comprising the amino acid sequence of SEQ ID NO:69 or 91 linked to the light chain variable domain. In some embodiments, a recombinant fusion protein includes (a) an antigen-binding fragment that binds to serum albumin, including a heavy chain variable domain including a heavy chain complementarity-determining region (HCDR) comprising of amino acid sequences of SEQ ID NOS:35, 36, and 37, a light chain variable domain including a light chain complementarity-determining region (LCDR) comprising of amino acid sequences of SEQ ID NOS:52, 53, and 54, a heavy chain constant domain comprising of an amino acid sequence of SEQ ID NO:68 linked to the heavy chain variable domain, and a light chain constant domain comprising of an amino acid sequence of SEQ ID NO:69 or 91 linked to the light chain variable domain, and (b) hFSH including an hFSH alpha subunit and an hFSH beta subunit, wherein the hFSH alpha subunit and the hFSH beta subunit are linked to the terminal regions of the antigen-binding fragment that binds to serum albumin.

Also disclosed herein are nucleic acid molecules encoding any of the recombinant fusion proteins disclosed herein.

Further disclosed herein are expression vectors comprising any of the nucleic acid molecules disclosed herein.

Disclosed herein are cells transformed with any of the expression vectors disclosed herein.

Disclosed herein are compositions comprising any of the recombinant fusion proteins disclosed herein. Also disclosed herein are pharmaceutical compositions comprising any of the compositions disclosed herein and a pharmaceutically acceptable carrier. Also disclosed are kits comprising any of the compositions disclosed herein and a label comprising instructions for a use.

Disclosed herein are methods of inducing superovulation of a subject in need thereof, comprising applying the pharmaceutical composition disclosed herein to in vitro fertilization-embryo transfer (IVF-ET), gamete intrafallopian transfer (GIFT), zygote intrafallopian transfer (ZIFT), intracytoplasmic sperm injection (ICSI), or in vitro fertilization. Disclosed herein are methods of treating anovulation, hypogonadism, or polycystic ovary syndrome in a subject in need thereof, comprising administering the pharmaceutical composition disclosed here to the subject.

Also disclosed herein are long-acting human follicle stimulating hormone (hFSH) compositions comprising the recombinant fusion protein disclosed herein as an active ingredient. In some embodiments, the long-acting hFSH composition is applied to in vitro fertilization-embryo transfer (IVF-ET), gamete intrafallopian transfer (GIFT), zygote intrafallopian transfer (ZIFT), intracytoplasmic sperm injection (ICSI), or in vitro fertilization, to induce superovulation of a subject. In some embodiments, the long-acting hFSH composition is used for treatment of anovulation, hypogonadism, or polycystic ovary syndrome.

In some embodiments, the composition is administered subcutaneously or intramuscularly to the subject.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A and 1B are diagrams schematically shows the structure of SAFA-FSH protein, wherein FIG. 1A shows the structure of SAFA-FSH-A protein and FIG. 1B shows the structure of SAFA-FSH-B;

FIGS. 2A and 2B are diagram showing a cleavage map of an expression vector for preparing SAFA-FSH protein, wherein FIG. 2A shows an expression vector for a heavy chain of the SAFA-FSH protein and FIG. 2B shows an expression vector for a light chain of the SAFA-FSH protein;

FIG. 3 shows the result of analyzing the size of SAFA-FSH protein by performing SDS-PAGE under a reducing condition with 1.175 μg/well and 2.35 μg/well (R), a non-reducing condition with heat applied (NR(B)), and a non-reducing condition without heat applied (NR(NB));

FIG. 4 shows the result of analyzing the purity of SAFA-FSH protein by performing SE-HPLC;

FIGS. 5A and 5B show the results of analyzing the isoelectric point of SAFA-FSH protein, wherein FIG. 5A shows the chromatographic peaks of the SAFA-FSH protein and FIG. 5B shows the bands of the SAFA-FSH protein;

FIG. 6 shows the molecular weight of the light chain and heavy chain in SAFA-FSH protein through capillary electrophoresis;

FIG. 7 shows the molecular weight of the light chain and heavy chain in SAFA-FSH protein by intact mass spectrometry;

FIG. 8 shows the result of N-terminal sequencing of the light chain and heavy chain in SAFA-FSH protein;

FIGS. 9A and 9B show the binding force between SAFA-FSH protein and human serum albumin, wherein FIG. 9A shows the binding force under pH 7.4 condition and FIG. 9B shows the binding force under pH 6.0 condition;

FIG. 10 shows the expression of a human follicle stimulating hormone receptor present on a surface of the human FSHR stabilizing cell line;

FIG. 11 shows the binding between SAFA-FSH protein and a human follicle stimulating hormone receptor;

FIGS. 12A and 12B the level of cAMP regulating ability of follicle stimulating hormone by addition of SAFA-FSH protein, wherein FIG. 12A shows the result obtained by adding the protein in a concentration of 10 ng/ml, and FIG. 12B shows the result obtained by adding the protein in a concentration of 303 pM;

FIG. 13 shows the change in blood concentration by using an animal model after intravenous administration of Gonal-F protein at a concentration of 88 μg/kg;

FIG. 14 shows the change in blood concentration by using an animal model after intravenous administration of SAFA-FSH protein at a concentration of 200 μg/kg;

FIG. 15 shows the change in blood concentration by using an animal model after intravenous administration of SAFA-FSH protein at a concentration of 600 μg/kg;

FIGS. 16A and 16B show the ovarian growth promoting effect by using an animal model after administration of SAFA-FSH protein, wherein FIG. 16A shows a result of quantitatively comparing changes in ovarian mass over time and FIG. 16B shows results of visually confirming changes in the ovaries with the naked eyes;

FIGS. 17A-17C show the efficacy of SAFA-FSH protein by using an animal model with hypogonadism, wherein FIG. 17A shows the result of comparing average serum testosterone levels in Weeks 1 to 4 from the beginning of the experiment, FIG. 15B shows the result of comparing average serum testosterone levels in Weeks 5 to 8 from the beginning of the experiment, and FIG. 17C shows the result of comparing average serum testosterone levels in Weeks 9 to 12 from the beginning of the experiment; and

FIGS. 18A-18D show the efficacy of SAFA-FSH protein using an animal model at Week 9 from the beginning of the experiment, wherein FIG. 18A shows the result of comparing the testicular weight, FIG. 18B shows the result of comparing the testis coefficient value (i.e., a ratio of testis/body weight), FIG. 18C shows the result of comparing the total number of sperms, and FIG. 18D shows the result of comparing the recovery levels of sperm count.

### DETAILED DESCRIPTION

The term “recombinant fusion protein” as used in the present specification refers to a protein in which two or more types of proteins are artificially linked. In some embodiments, the term refers to a protein in which the alpha (α) subunit and the beta (β) subunit of the FSH are linked to an antigen-binding fragment to serum albumin, i.e., an anti-serum albumin Fab antibody fragment. In some embodiments, the recombinant fusion protein can be obtained by expressing a fusion gene (expression vector), in which sequences of a gene encoding the FSH are linked to sequences of a gene encoding the antigen-binding fragment to anti-serum albumin, in a cell expression system. In such a recombinant fusion protein, the FSH and the anti-serum albumin Fab antibody fragment can be linked directly to each other or linked to each other through a connector such as a linker. In some embodiments, the recombinant fusion protein can be the one in which the FSH, a linker, a heavy chain including a heavy chain region of the antigen-binding fragment to serum albumin, and a light chain including a light chain region of the antigen-binding fragment to serum albumin are linked through non-covalent bonds.

As used herein, the term “serum albumin” is one of proteins constituting basic materials of cells and plays an important role in maintaining the osmotic pressure between blood vessels and tissues by allowing body fluids to stay in blood vessels. In addition, the term “antigen binding fragment against serum albumin” can refer to an anti-serum albumin antibody or an antigen binding fragment of the antibody molecule specifically binding to an epitope of serum albumin.

The term “antigen-binding fragment that binds to serum albumin” as used in the present specification refers to, as a fusion partner of the hFSH that is an active ingredient of the recombinant fusion protein, a functional unit that can contribute to significantly improving the pharmacokinetic properties of the recombinant fusion protein while maintaining the inherent biological activity or therapeutic efficacy of the hFSH. The antigen-binding fragment can be, for example, a human anti-serum albumin (SA) Fab, and details thereof are as disclosed in Korean Patent Registration No. 10-1576561 and U.S. Pat. Nos. 9,879,077 and 11,773,176, each incorporated herein by reference in its entirety.

The term “hFSH” is a glycoprotein hormone produced by the pituitary gland and secreted in the endocrine system, and has a dimer structure including of alpha and beta subunits. This term can be used interchangeably with the term “human ovary stimulating hormone”. The hFSH and the alpha and beta subunits can be obtained from the previously published database such as https://www.ncbi.nlm.nih.gov/ and can optionally include variants of the amino acid/nucleotide sequence having 80% or more of sequence identity/homology with the aforementioned amino acid/nucleotide sequence. Meanwhile, the FSH in women can stimulate follicles in the ovary during a follicular phase of a menstrual cycle, whereas the FSH in men can serve to stimulate sperm production in the testes. Therefore, the hFSH has been widely used as a sex hormone agent for improving or treating the infertility condition caused by dysfunction in the reproductive cycle or reproductive organs. However, despite the aforementioned therapeutic efficacy, the low production efficiency or short dosing interval due to short in vivo half-life are a burden to patients having a desire to receive infertility treatment.

Under such a technical background, as a result of putting much effort to develop a long-acting hFSH formulation, a recombinant fusion protein, in which an antigen-binding fragment that binds to serum albumin, SL335, is fused to a hFSH having a dimer structure including an FSH alpha subunit and an FSH beta subunit, was prepared. The improved pharmacokinetic properties and therapeutically effective effects on growth promotion of the ovary by the recombinant fusion protein were confirmed, thereby completing the present disclosure.

Disclosed herein are recombinant fusion proteins comprising (a) a human follicle stimulating hormone (hFSH) comprising an hFSH alpha subunit and an hFSH beta subunit and (b) an antigen binding fragment (Fab) that binds to serum albumin. In some embodiments, the hFSH alpha subunit and the hFSH beta subunit are linked to terminal regions of the Fab. In some embodiments, the hFSH alpha subunit and the hFSH beta subunit are independently linked to a terminus of the heavy chain constant 1 domain and a terminus of the light chain constant domain of the Fab. In some embodiments, the hFSH alpha subunit and the hFSH beta subunit are independently linked by a linker to a terminus of a heavy chain constant 1 domain and a terminus of a light chain constant domain of the Fab. In other embodiments, the hFSH alpha subunit is linked to a terminus of the light chain constant domain and the hFSH beta subunit is linked to a terminus of the heavy chain constant 1 domain of the Fab.

The Fab can comprise
a heavy chain comprising a heavy chain variable domain comprising
   (a) a heavy chain complementarity determining domain 1 (CDR1) comprising the amino acid sequence of SYGIS (SEQ ID NO:22),
      a heavy chain complementarity determining domain 2 (CDR2) comprising the amino acid sequence of WINTYSGGTKYAQKFQG (SEQ ID NO:23), and
      a heavy chain complementarity determining domain 3 (CDR3) comprising the amino acid sequence of LGHCQRGICSDALDT (SEQ ID NO:24);
   (b) a heavy chain CDR1 comprising the amino acid sequence of SYGIS (SEQ ID NO:22),
      a heavy chain CDR2 comprising the amino acid sequence of RINTYNGNTGYAQRLQG (SEQ ID NO:25), and
      a heavy chain CDR3 comprising the amino acid sequence of LGHCQRGICSDALDT (SEQ ID NO:24);
   (c) a heavy chain CDR1 comprising the amino acid sequence of NYGIH (SEQ ID NO:26),
      a heavy chain CDR2 comprising the amino acid sequence of SISYDGSNKYYADSVKG (SEQ ID NO:27), and
      a heavy chain CDR3 comprising the amino acid sequence of DVHYYGSGSYYNAFDI (SEQ ID NO:28);
   (d) a heavy chain CDR1 comprising the amino acid sequence of SYAMS (SEQ ID NO:29),
      a heavy chain CDR2 comprising the amino acid sequence of VISHDGGFQYYADSVKG (SEQ ID NO:30), and
      a heavy chain CDR3 comprising the amino acid sequence of AGWLRQYGMDV (SEQ ID NO:31);
   (e) a heavy chain CDR1 comprising the amino acid sequence of AYWIA (SEQ ID NO:32),
      a heavy chain CDR2 comprising the amino acid sequence of MIWPPDADARYSPSFQG (SEQ ID NO:33), and
      a heavy chain CDR3 comprising the amino acid sequence of LYSGSYSP (SEQ ID NO:34); or
   (f) a heavy chain CDR1 comprising the amino acid sequence of AYSMN (SEQ ID NO:35),
      a heavy chain CDR2 comprising the amino acid sequence of SISSSGRYIHYADSVKG (SEQ ID NO:36), and
      a heavy chain CDR3 comprising the amino acid sequence of ETVMAGKALDY (SEQ ID NO:37); and
a light chain comprising a light chain variable domain comprising
   (g) a light chain CDR1 comprising the amino acid sequence of RASQSISRYLN (SEQ ID NO:38),
      a light chain CDR2 comprising the amino acid sequence of GASRLES (SEQ ID NO:39), and
      a light chain CDR3 comprising the amino acid sequence of QQSDSVPVT (SEQ ID NO:40);
   (h) a light chain CDR1 comprising the amino acid sequence of RASQSISSYLN (SEQ ID NO:41),
      a light chain CDR2 comprising the amino acid sequence of AASSLQS (SEQ ID NO:42), and
      a light chain CDR3 comprising the amino acid sequence of QQSYSTPPYT (SEQ ID NO:43);
   (i) a light chain CDR1 comprising the amino acid sequence of RASQSIFNYVA (SEQ ID NO:44),
      a light chain CDR2 comprising the amino acid sequence of DASNRAT (SEQ ID NO:45), and
      a light chain CDR3 comprising the amino acid sequence of QQRSKWPPTWT (SEQ ID NO:46);
   (j) a light chain CDR1 comprising the amino acid sequence of RASETVSSRQLA (SEQ ID NO:47),
      a light chain CDR2 comprising the amino acid sequence of GASSRAT (SEQ ID NO:48), and
      a light chain CDR3 comprising the amino acid sequence of QQYGSSPRT (SEQ ID NO:49);
   (k) a light chain CDR1 comprising the amino acid sequence of RASQSVSSSSLA (SEQ ID NO:50),
      a light chain CDR2 comprising the amino acid sequence of GASSRAT (SEQ ID NO:48), and
      a light chain CDR3 comprising the amino acid sequence of QKYSSYPLT (SEQ ID NO:51); or
   (l) a light chain CDR1 comprising the amino acid sequence of RASQSVGSNLA (SEQ ID NO:52),
      a light chain CDR2 comprising the amino acid sequence of GASTGAT (SEQ ID NO:53), and
      a light chain CDR3 comprising the amino acid sequence of QQYYSFLAKT (SEQ ID NO:54).

In some embodiments, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. In some embodiments, the light chain variable domain comprises an amino acid sequence having at least 90% identity to SEQ ID NO:61, 62, 63, 64, 65, 66, or 67. In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence having at least 90% identity to SEQ ID NO:68. In some embodiments, the light chain constant domain comprises an amino acid sequence having at least 90% identity to SEQ ID NO:69 or 91.

In some embodiments, the heavy chain of the Fab comprises an amino acid sequence of SEQ ID NO:6. In some embodiments, the fusion protein comprises an amino acid sequence of SEQ ID NO:13 and an amino acid sequence of SEQ ID NO:6.

In some embodiments of the recombinant proteins disclosed herein, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90%, at least 93%, at least 95%, at least 96%, at least 97%, at least 98%, at least 99%, or 100% identity to SEQ ID NO:55, 56, 57, 58, 59, or 60.

In some embodiments, the light chain variable domain comprises an amino acid sequence having at least 90%, at least 93%, at least 95%, at least 96%, at least 97%, at least 98%, at least 99%, or 100% identity to SEQ ID NO:61, 62, 63, 64, 65, 66, or 67.

In some embodiments, the heavy chain variable domain comprises the amino acid sequence of SEQ ID NO:55, 56, 57, 58, 59, or 60, and the light chain variable domain comprises the amino acid sequence of SEQ ID NO:61, 62, 63, 64, 65, 66 or 67.

In some embodiments, the Fab comprises a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60, and a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, or 66 or 67, respectively, or in any combinations of heavy chain variable domain and light chain variable domain disclosed herein. For example, the Fab can comprise a heavy chain variable domain comprising an amino acid sequence having at least 90%, at least 93%, at least 95%, at least 96%, at least 97%, at least 98%, at least 99%, or 100% identity to SEQ ID NO:60 and a light chain variable domain comprising an amino acid sequence having at least 90%, at least 93%, at least 95%, at least 96%, at least 97%, at least 98%, at least 99%, or 100% identity to SEQ ID NO:67.

In some embodiments, the heavy chain constant domain comprises an amino acid sequence having at least 90%, at least 93%, at least 95%, at least 96%, at least 97%, at least 98%, at least 99%, or 100% identity to SEQ ID NO:68.

In some embodiments, the light chain constant domain comprises an amino acid sequence having at least 90%, at least 93%, at least 95%, at least 96%, at least 97%, at least 98%, at least 99%, or 100% identity to SEQ ID NO:69 or 91.

In some embodiments, the recombinant fusion protein can comprise a heavy chain comprising an amino acid sequence having at least 90%, at least 93%, at least 95%, at least 96%, at least 97%, at least 98%, at least 99%, or 100% identity to SEQ ID NO:19. In some embodiments, the Fab comprises a heavy chain domain comprising an amino acid sequence having at least 90%, at least 93%, at least 95%, at least 96%, at least 97%, at least 98%, at least 99%, or 100% identity to SEQ ID NO:6 (V_{H}-C_{H1} domain). In some embodiments, the Fab comprises a light chain domain comprising an amino acid sequence having at least 90%, at least 93%, at least 95%, at least 96%, at least 97%, at least 98%, at least 99%, or 100% identity to SEQ ID NO:13 (V_{L}-C_{κ} domain).

In some embodiments, the recombinant fusion protein can comprise a heavy chain comprising an amino acid sequence having at least 90%, at least 93%, at least 95%, at least 96%, at least 97%, at least 98%, at least 99%, or 100% identity to SEQ ID NO:19; and a light chain comprising an amino acid sequence having at least 90%, at least 93%, at least 95%, at least 96%, at least 97%, at least 98%, at least 99%, or 100% identity to SEQ ID NO:13. The recombinant protein can have significantly improved pharmacokinetic properties while maintaining the intrinsic biological activity of the FSH.

In some embodiments, the Fab comprises a heavy chain domain comprising an amino acid sequence having at least 90%, at least 93%, at least 95%, at least 96%, at least 97%, at least 98%, at least 99%, or 100% identity to SEQ ID NO:6 (V_{H}-C_{H1} domain) and a light chain domain comprising an amino acid sequence having at least 90%, at least 93%, at least 95%, at least 96%, at least 97%, at least 98%, at least 99%, or 100% identity to SEQ ID NO:13 (V_{L}-C_{κ} domain).

In some embodiments, the variable domain of the heavy chain (VH) can include a heavy chain complementarity-determining region (HCDR) comprising of amino acid sequences of SEQ ID NOS:35, 36, 37. For example, the variable domain of the heavy chain (VH) can include an amino acid sequence of SEQ ID NO:60 or an amino acid sequence having at least 80%, 85%, 90%, 95%, 99% or more sequence identity with the amino acid sequence of SEQ ID NO:60. The C-terminus of the variable domain of the heavy chain (VH) can be linked to the constant 1 domain of the heavy chain (CH1). Here, the constant 1 domain of the heavy chain (CH1) can include an amino acid sequence of SEQ ID NO:68, and for example, can include a sequence having at least 80%, 85%, 90%, 95%, 99% or more sequence identity with the amino acid sequence of SEQ ID NO:68, but embodiments are not particularly limited thereto.

In some embodiments, the variable domain of the light chain (VL) can include a light chain complementarity-determining region (LCDR) comprising of amino acid sequences of SEQ ID NOS:52, 53, and 54. For example, the variable domain of the light chain (VL) can include an amino acid sequence of SEQ ID NO:67 or an amino acid sequence having at least 80%, 85%, 90%, 95%, 99% or more sequence identity with the amino acid sequence of SEQ ID NO:67. The C-terminus of the variable domain of the light chain (VL) can be linked to the constant domain of the light chain (CL), wherein the light chain constant domain (CL) can include an amino acid sequence of SEQ ID NO:69 or 91, and for example, can include a sequence having at least 80%, 85%, 90%, and 95% or more sequence identity with the amino acid sequence of SEQ ID NO:69 or 91, but embodiments are not particularly limited thereto.

The term “identity” as used in the present specification refers to the overall relevance between polymer molecules, such as nucleic acids (e.g., DNA molecules and/or RNA molecules), and/or between polypeptides. For example, polypeptides are considered “substantially identical” when amino acid sequences thereof have at least 60%, 65%, 70%, 75%, 80%, 85%, 90%, 95%, or 99% identity. Calculating the percent identity of two nucleic acid or polypeptide sequences can be performed by, for example, aligning two sequences for the optimal comparison purposes (e.g., a gap can be introduced in one or both of first and second sequences for the optimal alignment, and non-identical sequences can be ignored for the comparison purposes). For example, the length of a sequence aligned for the comparison purposes is at least 60%, at least 70%, at least 80%, at least 90%, at least 95%, or substantially 100% of the length of a reference sequence. Subsequently, the nucleic acid or polypeptide sequences at corresponding positions are then compared. Determination of the percent identity between the two sequences and comparison of these sequences can be accomplished by using mathematical algorithms. As is well known to those skilled in the art, the amino acid or nucleic acid sequences can be compared by using any of a variety of algorithms including those available in commercial computer programs, specifically BLASTN for nucleotide sequences, and BLASTP, gapped BLAST, and PSIBLAS for amino acid sequences.

In some embodiments, the hFSH alpha subunit and the hFSH beta subunit can be linked to the terminal regions of the antigen-binding fragment that binds to serum albumin. For example, the hFSH alpha subunit and the hFSH beta subunit can be linked to the C-terminus or N-terminus of the antigen-binding fragment that binds to serum albumin. The hFSH alpha subunit and the hFSH beta subunit can independently be linked to the terminus of the constant 1 domain of the heavy chain (CH1) and the terminus of the constant domain of the light chain (CL), and more specifically, the N-termini of the hFSH alpha subunit and the hFSH beta subunit can independently be linked to the C-terminus of the constant 1 domain of the heavy chain (CH1) and to the C-terminus of the constant domain of the light chain (LC). More specifically, (1) the hFSH alpha subunit can be linked to the terminus, e.g., C-terminus, of the constant domain of the light chain (CL), and the hFSH beta subunit can be linked to the terminus, i.e., C-terminus, of the constant 1 domain of the heavy chain (CH1), or (2) the hFSH alpha subunit can be linked to the terminus, i.e., C-terminus, of the constant 1 domain of the heavy chain (CH1), and the hFSH beta subunit can be linked to the terminus, e.g., C-terminus, of the constant domain of the light chain (CL).

In some embodiments, the FSH alpha comprises an amino acid sequence having at least 90%, at least 93%, at least 95%, at least 96%, at least 97%, at least 98%, at least 99%, or 100% identity to SEQ ID NO:1. The FSH alpha can comprise an amino acid sequence having at least 90% identity to SEQ ID NO:1. In some embodiments, the FSH alpha comprises an amino acid sequence of SEQ ID NO:1. In some embodiments, a nucleic acid molecule encoding the FSH alpha comprises a nucleotide sequence of SEQ ID NO:2 or 3.

In some embodiments, the FSH beta comprises an amino acid sequence having at least 90%, at least 93%, at least 95%, at least 96%, at least 97%, at least 98%, at least 99%, or 100% identity to SEQ ID NO:4. The FSH beta can comprise an amino acid sequence having at least 90% identity to SEQ ID NO:4. In some embodiments, the FSH beta comprises an amino acid sequence of SEQ ID NO:4. In some embodiments, a nucleic acid molecule encoding the FSH beta comprises a nucleotide sequence of SEQ ID NO:5 or 9.

In some embodiments, in the fusion proteins disclosed herein, the heavy chain and the light chain of the Fab are bound by a noncovalent bond.

In some embodiments, the antigen-binding fragment that binds to serum albumin can be linked to the aforementioned hFSH, e.g., the hFSH alpha and beta subunits, through a linker. For example, the linker can link the hFSH subunits to any one region selected from the C-terminus of the constant 1 domain of the heavy chain (CH1), the N-terminus of the variable domain of the heavy chain (VH), the C-terminus of the constant domain of the light chain (CL), and the N-terminus of the variable domain of the light chain (VL). In addition, the linker can be changed for use as necessary.

For example, the linker can be a polypeptide comprising 5 to 400 amino acids, 5 to 200 amino acids, or 5 to 200 amino acids. Such a peptide linker can include Gly, Asn and Ser residues, and can also include neutral amino acids such as Thr and Ala. The amino acid sequences suitable for the peptide linker are known in the art. In addition, the number of copies “n” can be adjusted in consideration of linker optimization to achieve proper separation between functional moieties or to maintain essential inter-moiety interactions. Other linkers are known in the art, and for example, a G-S linker, in which not only a polar amino acid residue is added to improve water solubility, but also amino acid residues, such as T and A, are added to maintain flexibility, can be used.

Therefore, the linker can be a flexible linker containing G, S, and/or T, A residues. The linker can have a general formula of (GpSs)ₙ or (SpGs)ₙ, wherein, independently, p is an integer of 1 to 10, s is 0 or an integer of 0 to 10, p+s is an integer of 20 or less, and n is an integer of 1 to 20. More specifically, examples of the linker can include (GGGGS)ₙ (SEQ ID NO:72), (SGGGG)ₙ (SEQ ID NO:73), (SRSSG)ₙ(SEQ ID NO:74), (SGSSC)ₙ(SEQ ID NO:75), (GKSSGSGSESKS)ₙ (SEQ ID NO:76), (RPPPPC)ₙ (SEQ ID NO:77), (SSPPPPC)ₙ (SEQ ID NO:78), (GSTSGSGKSSEGKG)ₙ (SEQ ID NO:79), (GSTSGSGKSSEGSGSTKG)ₙ (SEQ ID NO:80), (GSTSGSGKPGSGEGSTKG)ₙ (SEQ ID NO:81), or (EGKSSGSGSESKEF)ₙ (SEQ ID NO:82), wherein n can be an integer of 1 to 20, or 1 to 10.

In addition, the linker can comprise an amino acid sequence of SEQ ID NO:85 or 88, but embodiments are not particularly limited thereto.

In some embodiments, the linker comprises 1 to 50 amino acids. In some embodiments, the linker comprises an amino acid sequence of any one of SEQ ID NOS:16, 70-85, and 88. In some embodiments, the recombinant fusion protein can include: i) a heavy chain including a heavy chain fragment SL335 of SEQ ID NO:6, a linker of SEQ ID NO:88, and an FSH alpha subunit of SEQ ID NO:1; and a light chain including a light chain fragment SL335 of SEQ ID NO:91, a linker of SEQ ID NO:85, and an FSH beta subunit of SEQ ID NO:4, or ii) a heavy chain including a heavy chain fragment SL335 of SEQ ID NO:6, a linker of SEQ ID NO:85, and an FSH beta subunit of SEQ ID NO:4; and a light chain including a light chain fragment SL335 of SEQ ID NO:91, a linker of SEQ ID NO:88, and an FSH alpha subunit of SEQ ID NO:1. The recombinant fusion protein can comprise, for example, amino acid sequences of SEQ ID NOS:19 and 21, or amino acid sequences of SEQ ID NOS:95 and 97. The recombinant fusion protein can have significantly improved pharmacokinetic properties while maintaining the inherent biological activity of the hFSH.

In some embodiments, recombinant fusion proteins (SAFA-FSH A and SAFA-FSH B) are prepared, including: the antigen-binding fragment that binds to serum albumin; and the hFSH subunits each fused to the C-terminus of each of the constant 1 domain of the heavy chain (CH1) and the constant domain of the light chain (CL). As the recombinant fusion protein retains the biological activity of each factor, specifically, binding ability to each of human serum albumin and FSH receptors, the half-life of in vivo loss can be extended, and the therapeutic efficacy such as growth promotion of the ovary can be improved.

In some embodiments, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and

wherein the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. In some embodiments, the heavy chain variable domain comprises the amino acid sequence of SEQ ID NO:60.

In some embodiments, the light chain variable domain comprises an amino acid sequence having at least 90% identity to SEQ ID NO:61, 62, 63, 64, 65, 66, or 67. In some embodiments, the light chain variable domain comprises the amino acid sequence of SEQ ID NO:67.

In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence.

In some embodiments, the light chain constant domain comprises an amino acid sequence having at least 90% identity to SEQ ID NO:69 or 91.

In some embodiments, the Fab comprises a heavy chain comprising the amino acid sequence of SEQ ID NO:6 and a light chain comprising the amino acid sequence of SEQ ID NO:13. In some embodiments, the Fab comprises a heavy chain constant 1 domain comprising the amino acid sequence of SEQ ID NO:68 linked to the heavy chain variable domain; and a light chain constant domain comprising the amino acid sequence of SEQ ID NO:69 or 91 linked to the light chain variable domain.

An antigen binding fragment of an antibody or an antibody fragment refers to a fragment retaining an antigen-binding function, and includes Fab, F(ab′), F(ab′)2, Fv, etc. Fab of the antibody fragments has a structure including variable regions of a light chain and a heavy chain, a constant region of the light chain, and a constant region (CH) of the heavy chain with one antigen-binding site. Fab′ differs from Fab in that it has a hinge region containing one or more cysteine residues at the C-terminal of the heavy chain CH domain. F(ab′)2 antibody is produced when the cysteine residue of the hinge region of Fab′ forms a disulfide bond. Recombinant techniques for generating Fv fragments with minimal antibody fragments having only a heavy chain variable region and a light chain variable region are described in PCT International Publication Nos. WO88/10649, WO88/106630, WO88/07085, WO88/07086, and WO88/09344. In a two-chain Fv, a heavy chain variable region and a light chain variable region are connected via a non-covalent bond. In a single chain Fv (scFv), a heavy chain variable region and a light chain variable region are generally connected via a peptide linker by a covalent bond or directly at the C-terminal. Thus, the single chain Fv (scFv) can have a structure such as a dimer, like the two-chain Fv. Such an antibody fragment can be obtained using a protein hydrolyzing enzyme (for example, when a whole antibody is cleaved with papain, Fab can be obtained, and when a whole antibody is cleaved with pepsin, F(ab′)2 fragment can be obtained), and it can also be produced through a recombinant gene technology.

In some embodiments, the antigen binding fragment against serum albumin can include a heavy chain region comprising an amino acid sequence of SEQ ID NO:6; and a light chain region comprising an amino acid sequence of SEQ ID NO:13. In some embodiments, nucleic acid molecule encoding the heavy chain region comprising the amino acid sequence of SEQ ID NO:6 can have a nucleotide sequence of SEQ ID NO:11 or 12. In some embodiments, nucleic acid molecule encoding the light chain region comprising the amino acid sequence of SEQ ID NO:13 can have a nucleotide sequence of SEQ ID NO:14 or 15.

As used herein, the terms “antibody” and “antibodies” are terms of art and can be used interchangeably herein and refer to a molecule with an antigen-binding site that specifically binds an antigen. Antibodies can include, e.g., monoclonal antibodies, recombinantly produced antibodies, human antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multispecific antibodies.

Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule.

As used herein, the terms “bioeffector moiety,” “antigen-binding domain,” “antigen-binding region,” “antigen-binding site,” and similar terms refer to the portions of the recombinant protein that comprises the amino acid residues that confer on the recombinant protein its specificity for the antigen (e.g., the complementarity determining regions (CDR)). The antigen-binding region can be derived from any animal species, such as feline, rodents (e.g., mouse, rat, or hamster) and humans.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody. The terms “VH” and “VH domain” are used interchangeably to refer to the heavy chain variable region of an antibody.

As used herein, the term “heavy chain (HC or CH)” refers to both a full-length heavy chain and a fragment thereof, the full-length heavy chain including a variable region domain VH including an amino acid sequence having a sufficient variable region (VR) sequence to confer specificity for an antigen and three constant region domains CH1, CH2, and CH3. As used herein, the term “light chain (LC or CL)” refers to both a full-length light chain and a fragment thereof, the full-length light chain including a variable region domain VL including an amino acid sequence having a sufficient VR sequence to confer specificity for an antigen and a constant region domain CL.

The heavy chain constant domain and the light chain constant domain can be derived from an IgG1 antibody constant domain, and in any one or more thereof, cysteine which is an amino acid used in a disulfide bond between the light chain and the heavy chain domain can be conserved or deleted or substituted with an amino acid residue other than cysteine. For example, the heavy chain constant domain can comprise an amino acid sequence having at least 90%, at least 93%, at least 95%, at least 96%, at least 97%, at least 98%, at least 99%, or 100% identity to SEQ ID NO:68, and the light chain constant domain can comprise an amino acid sequence having at least 90%, at least 93%, at least 95%, at least 96%, at least 97%, at least 98%, at least 99%, or 100% identity to SEQ ID NO:69 or 91. The deletion or substitution of cysteine in the domain can contribute to improving an expression level of the recombinant protein in transformed cells during a process of producing the above-mentioned recombinant protein. In some embodiments, (i) one or more cysteines in the heavy chain constant domain and/or (ii) one or more cysteines in the light chain constant domain that is/are located in an interchain disulfide bond between the light chain and the heavy chain is/are conserved, deleted, and/or substituted with an amino acid residue other than cysteine.

As used herein, the term “constant region” or “constant domain” are interchangeable and have its meaning common in the art. The constant region is an antibody portion, e.g., a carboxyl terminal portion of a light and/or heavy chain, which is not directly involved in binding of an antibody to an antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (C_{κ}) or lambda (Cλ) based on the amino acid sequence of the constant domains. Light chain amino acid sequences are well known in the art. In specific embodiments, the light chain is a human light chain.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore® or KinExA.

In some embodiments, the binding affinity of the recombinant fusion proteins disclosed herein has a binding affinity that is at least 1.5-fold, at least 2-fold, at least 3-fold, at least 4-fold, at least 5-fold, at least 6-fold, at least 7-fold, at least 8-fold, at least 9-fold, at least 10-fold, or any ranges therein higher than that of human FSH, e.g., 2-fold to 10-fold higher.

As used herein, an “epitope” is a term in the art and refers to a localized region of an antigen to which an antibody can specifically bind. An epitope can be, e.g., contiguous amino acids of a polypeptide (linear or contiguous epitope) or an epitope can, e.g., come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligopeptide scanning assays, and/or mutagenesis mapping (e.g., site-directed mutagenesis mapping). For X-ray crystallography, crystallization can be accomplished using any of the known methods in the art (e.g., Giegé R et al., (1994) Acta Crystallogr D Biol Crystallogr 50(Pt 4):339-350; McPherson A (1990) Eur J Biochem 189:1-23; Chayen N E (1997) Structure 5:1269-1274; McPherson, A. (1976) J. Biol. Chem. 251:6300-6303). Antibody:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1):37-60; Bricogne G (1997) Meth Enzymol 276A:361-423, ed Carter C W; Roversi P et al., (2000) Acta Crystallogr D Biol Crystallogr 56 (Pt 10):1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques. In some embodiments, the epitope of an antibody is determined using alanine scanning mutagenesis studies.

As used herein, the terms “immunospecifically binds,” “immunospecifically recognizes,” “specifically binds,” and “specifically recognizes” are analogous terms in the context of antibodies and refer to molecules that bind to an antigen (e.g., epitope, immune complex, or binding partner of an antigen-binding site) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In some embodiments, molecules that immunospecifically bind to an antigen bind to the antigen with a K_{A} that is at least 2 logs, 2.5 logs, 3 logs, 4 logs or greater than the K_{A} when the molecules bind to another antigen.

In some embodiments, molecules that immunospecifically bind to an antigen do not cross react with other proteins under similar binding conditions. In some embodiments, molecules that immunospecifically bind to an antigen do not cross react with other proteins. In some embodiments, provided herein are recombinant proteins that bind to a specified antigen with higher affinity than to another unrelated antigen. In certain embodiments, provided herein is a recombinant protein that binds to a specified antigen (e.g., human serum albumin) with a 20%, 25%, 30%, 35%, 40%, 45%, 50%, 55%, 60%, 65%, 70%, 75%, 80%, 85%, 90%, 95% or higher affinity than to another, unrelated antigen as measured by, e.g., a radioimmunoassay, surface plasmon resonance, or kinetic exclusion assay. In some embodiments, the extent of binding of a recombinant protein described herein to an unrelated, protein is less than 10%, 15%, or 20% of the binding of the antibody to the specified antigen as measured by, e.g., a radioimmunoassay.

In some embodiments, provided herein are recombinant proteins that bind to an antigen of various species, such as feline, rodents (e.g., mouse, rat, or hamster) and humans. In some embodiments, provided herein are recombinant proteins that bind to a human antigen with higher affinity than to another species of the antigen. In certain embodiments, provided herein are recombinant proteins that bind to a human antigen with a 5%, 10%, 15%, 20%, 25%, 30%, 35%, 40%, 45%, 50%, 55%, 60%, 65%, 70% or higher affinity than to another species as measured by, e.g., a radioimmunoassay, surface plasmon resonance, or kinetic exclusion assay. In some embodiments, the recombinant proteins described herein, which bind to a human antigen, will bind to another species of the antigen protein with less than 10%, 15%, or 20% of the binding of the antibody to the human antigen protein as measured by, e.g., a radioimmunoassay, surface plasmon resonance, or kinetic exclusion assay.

As used herein, the term “host cell” can be any type of cell, e.g., a primary cell, a cell in culture, or a cell from a cell line. In embodiments, the term “host cell” refers to a cell transfected with a nucleic acid molecule and the progeny or potential progeny of such a cell. Progeny of such a cell cannot be identical to the parent cell transfected with the nucleic acid molecule, e.g., due to mutations or environmental influences that can occur in succeeding generations or integration of the nucleic acid molecule into the host cell genome.

In certain aspects, a recombinant protein described herein can be described by its VL domain alone, or its VH domain alone, or by its 3 VL CDRs alone, or its 3 VH CDRs alone. See, e.g., Rader C et al., (1998) PNAS 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352:624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45:572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In certain aspects, provided herein are recombinant proteins that specifically bind to serum albumin (e.g., human serum albumin) and comprise the Chothia VL CDRs of a VL. In certain aspects, provided herein are antibodies that specifically bind to serum albumin (e.g., human serum albumin) and comprise the Chothia VH CDRs of a VH. In certain aspects, provided herein are antibodies that specifically bind to serum albumin (e.g., human serum albumin) and comprise the Chothia VL CDRs of a VL and comprise the Chothia VH CDRs of a VH. In certain embodiments, antibodies that specifically bind to serum albumin (e.g., human serum albumin) comprise one or more CDRs, in which the Chothia and Kabat CDRs have the same amino acid sequence. In certain embodiments, provided herein are antibodies that specifically bind to serum albumin and comprise combinations of Kabat CDRs and Chothia CDRs.

In certain aspects, the CDRs of an antibody can be determined according to the AbM numbering scheme, which refers AbM hypervariable regions that represent a compromise between the Kabat CDRs and Chothia structural loops and are used by Oxford Molecular's AbM antibody modeling software (Oxford Molecular Group, Inc.).

In some embodiments, the position of one or more CDRs along the VH (e.g., CDR1, CDR2, or CDR3) and/or VL (e.g., CDR1, CDR2, or CDR3) region of an antibody described herein can vary by one, two, three, four, five, or six amino acid positions so long as immunospecific binding to an antigen is maintained (e.g., substantially maintained, e.g., at least 50%, at least 60%, at least 70%, at least 80%, at least 90%, at least 95%). For example, the position defining a CDR of an antibody described herein can vary by shifting the N-terminal and/or C-terminal boundary of the CDR by one, two, three, four, five, or six amino acids, relative to the CDR position of an antibody described herein, so long as immunospecific binding to the antigen(s) is maintained (e.g., substantially maintained, e.g., at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of an antibody described herein can vary (e.g., be shorter or longer) by one, two, three, four, five, or more amino acids, so long as immunospecific binding to the antigen(s) is maintained (e.g., substantially maintained, e.g., at least 50%, at least 60%, at least 70%, at least 80%, at least 90%, at least 95%).

In some embodiments, a VL CDR1, VL CDR2, VL CDR3, VH CDR1, VH CDR2, and/or VH CDR3 described herein can be one, two, three, four, five or more amino acids shorter than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, a VL CDR1, VL CDR2, VL CDR3, VH CDR1, VH CDR2, and/or VH CDR3 described herein can be one, two, three, four, five or more amino acids longer than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the carboxy terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In some embodiments, the carboxy terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). Any method known in the art can be used to ascertain whether immunospecific binding to the antigen(s) is maintained, e.g., the binding assays and conditions described in the “Examples” section herein.

The percent identity between two sequences can be determined using techniques similar to those described above, with or without allowing gaps. In calculating percent identity, typically only exact matches are counted.

The recombinant proteins disclosed herein can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I) carbon (¹⁴C), sulfur (³⁵S), tritium (3H), indium (¹²¹In), and technetium (⁹⁹Tc); luminescent labels, such as luminol; and fluorescent labels, such as fluorescein and rhodamine, and biotin. Such labeled antibodies can be used to detect antigen proteins.

### Polynucleotides, Vectors, and Cells

According to some aspects of the disclosure, provided are a nucleic acid encoding the recombinant fusion protein, an expression vector including the nucleic acid, and a cell transformed with the expression vector.

Since the nucleic acid, the expression vector, and the transformed cell include or use the aforementioned recombinant fusion protein or a nucleic acid encoding the recombinant fusion protein as it is, descriptions of common contents therebetween will be omitted.

For example, a recombinant fusion protein can be produced by separating the nucleic acid encoding the recombinant fusion protein. Following the separation of the nucleic acid, the resultant nucleic acid can be inserted into a replicable vector to be further cloned (i.e., amplification of DNA) or to be further expressed. In this regard, some aspects provide a vector including the nucleic acid.

The term “nucleic acid” as used in the present specification comprehensively includes DNA (gDNA and cDNA) and RNA molecules, and the term “nucleotide”, which is the basic structural unit in a nucleic acid, includes not only natural nucleotides, but also analogs in which sugar or base sites are modified.

Nucleic acid is also construed to include a nucleotide sequence exhibiting substantial identity to the nucleotide sequence. The substantial identity refers to nucleotide sequences exhibiting at least 80% homology, more preferably at least 90% homology, and most preferably at least 95% homology, in the case where the nucleotide sequences of the present disclosure and other sequences are aligned so as to correspond as much as possible, and the aligned sequences are analyzed by using algorithms commonly used in the art.

DNA encoding the recombinant fusion protein can be readily separated or synthesized by using conventional procedures (e.g., by using an oligonucleotide probe capable of binding specifically to DNA encoding the recombinant fusion protein). Many vectors are available. Vector components generally include, but are not limited to, one or more of the following: a signal sequence, an origin of replication, one or more marker genes, an enhancer element, a promoter, and a transcription termination sequence.

The term “vector” as used in the present specification refers to a plasmid vector as a means for expressing a target gene in a host cell, and examples thereof include: a plasmid vector; a cosmid vector; a viral vector such as a bacteriophage vector, an adenoviral vector, a retroviral vector, and an adeno-associated viral vector and the like. In the vector, the nucleic acid encoding the recombinant fusion protein can operably be linked to a promoter.

The expression “operably linked” as used in the present specification refers to a functional linkage between a control sequence of nucleic acid expression (e.g., a promoter, a signal sequence, or an array at a binding site for a transcriptional regulator) and another nucleic acid sequence, whereby the control sequence regulates transcription and/or translation of the other nucleic acid sequence.

In some embodiments, when a prokaryotic cell is used as a host, a strong promoter capable of initiating transcription (e.g., tac promoter, lac promoter, lacUV5 promoter, lpp promoter, pLλ promoter, pRλ promoter, rac5 promoter, amp promoter, recA promoter, SP6 promoter, trp promoter, T7 promoter, etc.), a ribosome binding site for initiation of translation, and a transcription/translation termination sequence are typically included. In one or more embodiments, when a eukaryotic cell is used as a host, a promoter derived from the genome of mammalian cells (e.g., metallothionein promoter, 3-actin promoter, human hemoglobin promoter, and human muscle creatine promoter) or a promoter derived from mammalian viruses (e.g., adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), and promoter of Rouss sarcoma virus (RSV)) can be used, and these promoters generally have a polyadenylation sequence as a transcription termination sequence. In one or more embodiments, the vector can be fused with other sequences to facilitate purification of the recombinant fusion proteins expressed from the vector. Sequences to be fused can include, for example, glutathione S-transferase (Pharmacia, USA), maltose-binding protein (NEB, USA), FLAG (IBI, USA), and 6×His (hexahistidine; Qiagen, USA), and the like. The vector can include, as a selectable marker, antibiotic resistance genes commonly used in the art, and for example, genes resistant to ampicillin, gentamicin, carbenicillin, chloramphenicol, streptomycin, kanamycin, geneticin, neomycin, and tetracycline can be included.

Some aspects of the disclosure provides a cell transformed with the aforementioned vector. Cells used to produce the recombinant fusion protein of the disclosure can be prokaryotic cells, yeast cells, or higher eukaryotic cells, but embodiments are not particularly limited thereto. For use as prokaryotic host cells, Escherichia coli, strains of the genus Bacillus, such as B. subtilis and B. thuringiensis, the genus Streptomyces, the genus Pseudomonas (e.g., P putida), Proteus mirabilis, and the genus Staphylococcus (e.g., S. carnosus) can be used. However, animal cells are of greatest interest, and examples of useful host cell lines are COS-7, BHK, CHO (GS null CHO-K1), CHOK1, DXB-11, DG-44, CHO/-DHFR, CV1, COS-7, HEK293, BHK, TM4, VERO, HELA, MDCK, BRL 3A, WI-38, Hep G2, SK-Hep, MMT, TRI, MRC 5, FS4, 3T3, RIN, A549, PC12, K562, PER.C6, SP2/0, NS-0, U20S, or HT1080, but embodiments are not particularly limited thereto.

The term “transformation” as used in the present specification refers to a molecular biology technology in which a DNA chain fragment or a plasmid having foreign genes of a different kind from those of the original cell penetrates between cells and binds to the DNA existing in the original cell, thereby transforming the genetic traits of the original cell. The transformation can refer that an expression vector including the recombinant fusion protein gene is inserted into a host cell.

Disclosed herein are nucleic acid molecules encoding the recombinant proteins disclosed herein.

Disclosed herein are expression vectors comprising the nucleic acid molecules disclosed herein.

Disclosed herein are cells transformed with the expression vectors disclosed herein.

Since the nucleic acid, the expression vector, and the transformed cell include the above-described recombinant protein or the nucleic acid encoding the recombinant protein as it is, or they use the same, descriptions common thereto will be omitted.

For example, in some aspects, the recombinant protein can be produced by isolating the nucleic acid encoding the recombinant protein. The nucleic acid is isolated and inserted into a replicable vector to perform additional cloning (DNA amplification) or additional expression. On the basis of this, other aspects relate to a vector including the nucleic acid.

As used herein, the term “nucleic acid” or “nucleic acid molecule” comprehensively includes DNA (gDNA and cDNA) and RNA molecules, and nucleotides as basic units of the nucleic acid include not only natural nucleotides but also analogues having modified sugar or base moieties.

The nucleic acid is interpreted to include a nucleotide sequence showing substantial identity to the nucleotide sequence. Substantial identity means a nucleotide sequence showing at least 80% homology, more specifically at least 90% homology, and most specifically at least 95% homology, when the nucleotide sequence of the present disclosure and another optional sequence are aligned to correspond to each other as much as possible and the aligned sequences are analyzed using an algorithm commonly used in the art.

DNA encoding the recombinant protein is easily isolated or synthesized by using a common process (e.g., by using an oligonucleotide probe capable of specifically binding to the DNA encoding the recombinant protein). Many vectors are available. Vector components generally include, but are not limited to, one or more of the following: a signal sequence, an origin of replication, one or more marker genes, an enhancer element, a promoter, and a transcription termination sequence.

As used herein, the term “vector” includes, as a means to express a target gene in a host cell, plasmid vectors; cosmid vectors; viral vectors such as bacteriophage vectors, adenovirus vectors, retrovirus vectors, and adeno-associated virus vectors, etc. In the vector, the nucleic acid encoding the recombinant protein is operably linked to a promoter.

“Operably linked” refers to a functional linkage between a nucleic acid expression control sequence (e.g., a promoter, a signal sequence, an array of transcriptional regulatory factor binding sites) and another nucleic acid sequence, whereby the control sequence directs transcription and/or translation of another nucleic acid sequence.

When a prokaryotic cell is used as a host, a powerful promoter capable of directing transcription (e.g., tac promoter, lac promoter, lacUV5 promoter, lpp promoter, pLλ promoter, pRλ promoter, rac5 promoter, amp promoter, recA promoter, SP6 promoter, trp promoter and T7 promoter, etc.), a ribosome binding site for initiation of translation, and a transcription/translation termination sequence are generally included. For example, when a eukaryotic cell is used as a host, a promoter derived from the genome of a mammalian cell (e.g., metallothionein promoter, β-actin promoter, human hemoglobin promoter, and human muscle creatine promoter) or a promoter derived from mammalian viruses (e.g., adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), and promoter of Rous sarcoma virus (RSV)) can be used, and a polyadenylated sequence can be commonly used as the transcription termination sequence. In some cases, the vector can be fused with another sequence to facilitate purification of the recombinant protein expressed therefrom. The sequence to be fused includes, e.g., glutathione S-transferase (Pharmacia, USA), maltose binding protein (NEB, USA), FLAG (IBI, USA), 6×His (hexahistidine; Quiagen, USA), etc. The vector includes, as a selective marker, an antibiotic-resistant gene that is ordinarily used in the art, e.g., genes resistant against ampicillin, gentamycin, carbenicillin, chloramphenicol, streptomycin, kanamycin, geneticin, neomycin, and tetracycline.

In still other aspects, the present disclosure provides cells transformed with the above-mentioned vectors. The cells used to produce the recombinant protein of the present disclosure can be prokaryotic cells, yeast cells, or higher eukaryotic cells, but are not limited thereto. Prokaryotic host cells such as Escherichia coli, the genus bacillus strains such as Bacillus subtilis and Bacillus thuringiensis, Streptomyces, Pseudomonas (e.g., Pseudomonas putida), Proteus mirabilis and Staphylococcus (e.g., Staphylococcus carnosus) can be used. However, animal cells are most interested, and examples of the useful host cell line can include COS-7, BHK, CHO (GS null CHO-K1), CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, or HT1080, but are not limited thereto.

As used herein, the term “transformation” means a molecular biological technique that changes the genetic trait of a cell by a DNA chain fragment or plasmid which possesses a different type of foreign gene from that of the original cell, penetrates among the cells, and combines with DNA in the original cell. The transformation means insertion of the expression vector including the gene of the recombinant protein into a host cell.

Provided herein are nucleic acid molecules comprising a nucleotide sequence encoding a recombinant protein described herein (e.g., a variable light chain region and/or variable heavy chain region) that immunospecifically binds to an antigen, and vectors, e.g., vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In some embodiments, a nucleic acid molecule(s) encoding an antibody described herein is isolated or purified.

Provided herein are polynucleotides comprising nucleotide sequences encoding antibodies, which immunospecifically bind to an antigen polypeptide (e.g., human serum albumin) and comprises an amino acid sequence as described herein, as well as antibodies that compete with such antibodies for binding to an antigen polypeptide (e.g., in a dose-dependent manner), or which binds to the same epitope as that of such antibodies.

Provided herein are polynucleotides comprising a nucleotide sequence encoding the light chain or heavy chain of an antibody described herein. The polynucleotides can comprise nucleotide sequences encoding a light chain comprising the VL FRs and CDRs of antibodies described herein. The polynucleotides can comprise nucleotide sequences encoding a heavy chain comprising the VH FRs and CDRs of antibodies described herein.

Provided herein are polynucleotides comprising a nucleotide sequence encoding a recombinant protein comprising a Fab comprising three VH chain CDRs, e.g., containing VL CDR1, VL CDR2, and VL CDR3 of an antibody to human serum albumin described herein and three VH chain CDRs, e.g., containing VH CDR1, VH CDR2, and VH CDR3 of an antibody to human serum albumin described herein.

Provided herein are polynucleotides comprising a nucleotide sequence encoding a recombinant protein comprising a VL domain.

In certain embodiments, a polynucleotide described herein comprises a nucleotide sequence encoding a recombinant protein provided herein comprising a light chain variable region comprising an amino acid sequence described herein (e.g., SEQ ID NO:61, 62, 63, 64, 65, 66, or 67), wherein the antibody immunospecifically binds to serum albumin.

In certain embodiments, a polynucleotide described herein comprises a nucleotide sequence encoding an antibody provided herein comprising a heavy chain variable region comprising an amino acid sequence described herein (e.g., SEQ ID NO:55, 56, 57, 58, 59, or 60), wherein the antibody immunospecifically binds to serum albumin.

In specific aspects, provided herein are polynucleotides comprising a nucleotide sequence encoding an antibody comprising a light chain and a heavy chain, e.g., a separate light chain and heavy chain. With respect to the light chain, in some embodiments, a polynucleotide provided herein comprises a nucleotide sequence encoding a kappa light chain.

In other embodiments, a polynucleotide provided herein comprises a nucleotide sequence encoding a lambda light chain. In yet other embodiments, a polynucleotide provided herein comprises a nucleotide sequence encoding an antibody described herein comprising a human kappa light chain or a human lambda light chain. In some embodiments, a polynucleotide provided herein comprises a nucleotide sequence encoding an antibody, which immunospecifically binds to serum albumin, wherein the antibody comprises a light chain, and wherein the amino acid sequence of the VL domain can comprise the amino acid sequence set forth in SEQ ID NO:61, 62, 63, 64, 65, 66, or 67 and wherein the constant region of the light chain comprises the amino acid sequence of a kappa light chain constant region.

Also provided herein are polynucleotides encoding an antibody or a fragment thereof that are optimized, e.g., by codon/RNA optimization, replacement with heterologous signal sequences, and elimination of mRNA instability elements. Methods to generate optimized nucleic acids encoding an antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In certain embodiments, an optimized polynucleotide sequence encoding an antibody described herein or a fragment thereof (e.g., VL domain or VH domain) can hybridize to an antisense (e.g., complementary) polynucleotide of an unoptimized polynucleotide sequence encoding an antibody described herein or a fragment thereof (e.g., VL domain or VH domain). In specific embodiments, an optimized nucleotide sequence encoding an antibody described herein or a fragment hybridizes under high stringency conditions to antisense polynucleotide of an unoptimized polynucleotide sequence encoding an antibody described herein or a fragment thereof. In some embodiments, an optimized nucleotide sequence encoding an antibody described herein or a fragment thereof hybridizes under high stringency, intermediate or lower stringency hybridization conditions to an antisense polynucleotide of an unoptimized nucleotide sequence encoding an antibody described herein or a fragment thereof. Information regarding hybridization conditions has been described, see, e.g., US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies described herein and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17: 242-246), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or fragment thereof described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, e.g., to generate chimeric and humanized antibodies.

If a clone containing a nucleic acid encoding a particular antibody or fragment thereof is not available, but the sequence of the antibody molecule or fragment thereof is known, a nucleic acid encoding the immunoglobulin or fragment can be chemically synthesized or obtained from a suitable source (e.g., an antibody cDNA library or a cDNA library generated from, or nucleic acid, such as poly A+RNA, isolated from, any tissue or cells expressing the antibody, such as hybridoma cells selected to express an antibody described herein) by PCR amplification using synthetic primers capable of hybridizing to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding recombinant proteins described herein can be readily isolated and sequenced using conventional procedures (e.g., by using oligonucleotide probes that are capable of binding specifically to genes encoding the heavy and light chains of the recombinant proteins). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of recombinant proteins in the recombinant host cells.

To generate antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, e.g., by substituting the coding sequence for human heavy and light chain constant domains in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3.

Other aspects provide recombinant vectors comprising the gene encoding the FSH and the nucleic acid encoding the antigen binding fragment against serum albumin. Still other aspects provide a cell transformed with the vector.

Disclosed herein are nucleic acid molecules encoding a heavy chain region comprising an amino acid sequence having at least 90%, at least 93%, at least 95%, at least 96%, at least 97%, at least 98%, at least 99%, or 100% identity to SEQ ID NO:6. Disclosed herein are nucleic acid molecules encoding a light chain region comprising a nucleotide sequence having at least 90%, at least 93%, at least 95%, at least 96%, at least 97%, at least 98%, at least 99%, or 100% identity to SEQ ID NO:14 or 15.

Disclosed herein are nucleic acid molecules encoding a light chain region comprising an amino acid sequence having at least 90%, at least 93%, at least 95%, at least 96%, at least 97%, at least 98%, at least 99%, or 100% identity to SEQ ID NO:13. Disclosed herein are nucleic acid molecules encoding a light chain region comprising a nucleotide sequence having at least 90%, at least 93%, at least 95%, at least 96%, at least 97%, at least 98%, at least 99%, or 100% identity to SEQ ID NO:14 or 15.

In some embodiments, disclosed herein are nucleic acids, each encoding the heavy chain region of SEQ ID NO:6 and the light chain region of SEQ ID NO:13. In some embodiments, the nucleic acid encoding the heavy chain region of SEQ ID NO:6 can be represented by SEQ ID NO:7 or SEQ ID NO:8, and the nucleic acid encoding the light chain region of SEQ ID NO:13 can be represented by SEQ ID NO:14 or SEQ ID NO:15.

Further disclosed herein are expression vectors comprising:
(a) a promoter,
(b) a first nucleic acid molecule encoding a light chain that binds to serum albumin, and
(c) a second nucleic acid molecule encoding heavy chain and a bioactive effector moiety such as FSH and a linker,
wherein the promoter, the first nucleic acid sequence, and the second nucleic acid molecules are operably linked. The second nucleic acid molecule can encode 1, 2, 3, 4, 5, 6, or more bioactive effector moieties and linkers.

Also disclosed herein are expression vectors comprising:
(a) a promoter and
(b) a nucleic acid molecule encoding a heavy chain variable domain as disclosed herein and a heavy chain constant domain as disclosed herein.

Also disclosed herein are expression vectors comprising:
(a) a promoter and
(b) a nucleic acid molecule encoding a IL18BP as disclosed herein, a heavy chain variable domain as disclosed herein, and a heavy chain constant domain as disclosed herein.

Also disclosed herein are expression vectors comprising:
(a) a promoter and
(b) a nucleic acid molecule encoding a light chain variable domain as disclosed herein and a light chain constant domain as disclosed herein.

Also disclosed herein are expression vectors comprising:
(a) a promoter and
(b) a nucleic acid molecule encoding an FSH as disclosed herein, a light chain variable domain as disclosed herein, and a light chain constant domain as disclosed herein. One, two, three, or more expression vectors or nucleic acid molecules can be expressed to produce the desired recombinant proteins.

In some embodiments, a first nucleic acid molecule or vector comprises a nucleic acid sequence encoding a recombinant protein comprising an antigen binding fragment comprising a heavy chain, wherein the heavy chain comprises a heavy chain variable domain and a heavy chain constant domain, wherein the heavy chain variable domain comprises
(1) a heavy chain complementarity determining domain 1 (CDR1) comprising the amino acid sequence of SYGIS (SEQ ID NO:22),
   a heavy chain complementarity determining domain 2 (CDR2) comprising the amino acid sequence of WINTYSGGTKYAQKFQG (SEQ ID NO:23), and
   a heavy chain complementarity determining domain 3 (CDR3) comprising the amino acid sequence of LGHCQRGICSDALDT (SEQ ID NO:24);
(2) a heavy chain CDR1 comprising the amino acid sequence of SYGIS (SEQ ID NO:22),
   a heavy chain CDR2 comprising the amino acid sequence of RINTYNGNTGYAQRLQG (SEQ ID NO:25), and
   a heavy chain CDR3 comprising the amino acid sequence of LGHCQRGICSDALDT (SEQ ID NO:24);
(3) a heavy chain CDR1 comprising the amino acid sequence of NYGIH (SEQ ID NO:26),
   a heavy chain CDR2 comprising the amino acid sequence of SISYDGSNKYYADSVKG (SEQ ID NO:27), and
   a heavy chain CDR3 comprising the amino acid sequence of DVHYYGSGSYYNAFDI (SEQ ID NO:28);
(4) a heavy chain CDR1 comprising the amino acid sequence of SYAMS (SEQ ID NO:29),
   a heavy chain CDR2 comprising the amino acid sequence of VISHDGGFQYYADSVKG (SEQ ID NO:30), and
   a heavy chain CDR3 comprising the amino acid sequence of AGWLRQYGMDV (SEQ ID NO:31);
(5) a heavy chain CDR1 comprising the amino acid sequence of AYWIA (SEQ ID NO:32),
   a heavy chain CDR2 comprising the amino acid sequence of MIWPPDADARYSPSFQG (SEQ ID NO:33), and
   a heavy chain CDR3 comprising the amino acid sequence of LYSGSYSP (SEQ ID NO:34); or
(6) a heavy chain CDR1 comprising the amino acid sequence of AYSMN (SEQ ID NO:35),
   a heavy chain CDR2 comprising the amino acid sequence of SISSSGRYIHYADSVKG (SEQ ID NO:36), and
   a heavy chain CDR3 comprising the amino acid sequence of ETVMAGKALDY (SEQ ID NO:37).

Disclosed herein is a second nucleic acid molecule or vector comprises a nucleic acid sequence encoding a recombinant protein comprising an antigen binding fragment comprising a light chain, wherein the light chain comprises a light chain variable domain and a light chain constant domain, wherein the light chain variable domain comprises
(7) a light chain CDR1 comprising the amino acid sequence of RASQSISRYLN (SEQ ID NO:38),
   a light chain CDR2 comprising the amino acid sequence of GASRLES (SEQ ID NO:39), and
   a light chain CDR3 comprising the amino acid sequence of QQSDSVPVT (SEQ ID NO:40);
(8) a light chain CDR1 comprising the amino acid sequence of RASQSISSYLN (SEQ ID NO:41),
   a light chain CDR2 comprising the amino acid sequence of AASSLQS (SEQ ID NO:42), and
   a light chain CDR3 comprising the amino acid sequence of QQSYSTPPYT (SEQ ID NO:43);
(9) a light chain CDR1 comprising the amino acid sequence of RASQSIFNYVA (SEQ ID NO:44),
   a light chain CDR2 comprising the amino acid sequence of DASNRAT (SEQ ID NO:45), and
   a light chain CDR3 comprising the amino acid sequence of QQRSKWPPTWT (SEQ ID NO:46); (10) a light chain CDR1 comprising the amino acid sequence of RASETVSSRQLA (SEQ ID NO:47),
   a light chain CDR2 comprising the amino acid sequence of GASSRAT (SEQ ID NO:48), and
   a light chain CDR3 comprising the amino acid sequence of QQYGSSPRT (SEQ ID NO:49); (11) a light chain CDR1 comprising the amino acid sequence of RASQSVSSSSLA (SEQ ID NO:50),
   a light chain CDR2 comprising the amino acid sequence of GASSRAT (SEQ ID NO:48), and
   a light chain CDR3 comprising the amino acid sequence of QKYSSYPLT (SEQ ID NO:51); or (12) a light chain CDR1 comprising the amino acid sequence of RASQSVGSNLA (SEQ ID NO:52),
   a light chain CDR2 comprising the amino acid sequence of GASTGAT (SEQ ID NO:53), and
   a light chain CDR3 comprising the amino acid sequence of QQYYSFLAKT (SEQ ID NO:54).

For example, the nucleic acid molecule encoding FSH can be linked to the first or second nucleic acid molecule or vector described above.

In other embodiments, the first nucleic acid molecule can comprise a nucleic acid sequence encoding a Fab comprising: a heavy chain variable domain comprising (1) above and a light chain variable domain comprising (7) above; a heavy chain variable domain comprising (2) above and a light chain variable domain comprising (8) above; a heavy chain variable domain comprising (3) above and a light chain variable domain comprising (9) above; a heavy chain variable domain comprising (4) above and a light chain variable domain comprising (10) above; a heavy chain variable domain comprising (5) above and a light chain variable domain comprising (11) above; a heavy chain variable domain comprising (6) above and a light chain variable domain comprising (12) above; or any or all combinations of a heavy chain variable domain and a light chain variable domain described above. In some embodiments, the first nucleic acid molecule comprises a nucleic acid sequence encoding a Fab (SL335) comprising the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54. The first or second nucleic acid molecule can encode the FSH alpha or beta.

In other embodiments, a first nucleic acid molecule or vector comprises a nucleic acid sequence encoding a Fab comprising a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60. In some embodiments, a second nucleic acid molecule or vector comprises a nucleic acid sequence encoding a Fab comprising a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, 66, or 67. The nucleic acid molecule encoding FSH can be linked to the first or second nucleic acid molecule or vector.

In some embodiments, a first nucleic acid molecule or vector comprises a nucleic acid sequence encoding a Fab comprising a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60, and a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, or 66 or 67, respectively.

In some embodiments, the first nucleic acid molecule comprises a nucleic acid sequence encoding a Fab (SL335) comprising a heavy chain domain comprising an amino acid sequence having at least 90%, at least 93%, at least 95%, at least 96%, at least 97%, at least 98%, at least 99%, or 100% identity to SEQ ID NO:6 (V_{H}-C_{H1} domain) and a light chain domain comprising an amino acid sequence having at least 90%, at least 93%, at least 95%, at least 96%, at least 97%, at least 98%, at least 99%, or 100% identity to SEQ ID NO:13 (V_{L}—C_{κ} domain).

In some embodiments, the bioactive effector moiety is FSH. In some embodiments, a nucleic acid molecule encodes an FSH alpha protein comprises an amino acid sequence having at least 90%, at least 93%, at least 95%, at least 96%, at least 97%, at least 98%, at least 99%, or 100% identity to SEQ ID NO:1. In some embodiments, a nucleic acid molecule encoding the FSH alpha comprises a nucleotide sequence having at least 90%, at least 93%, at least 95%, at least 96%, at least 97%, at least 98%, at least 99%, or 100% identity to SEQ ID NO:2 or 3. For example, the first nucleic acid molecule can comprise a nucleotide sequence encoding the amino acid sequence having at least 90%, at least 93%, at least 95%, at least 96%, at least 97%, at least 98%, at least 99%, or 100% identity to one or more of SEQ ID NO:1, e.g., SEQ ID NO:2 or 3.

In some embodiments, a nucleic acid molecule encodes an FSH beta protein comprises an amino acid sequence having at least 90%, at least 93%, at least 95%, at least 96%, at least 97%, at least 98%, at least 99%, or 100% identity to SEQ ID NO:4. In some embodiments, a nucleic acid molecule encoding the FSH beta comprises a nucleotide sequence having at least 90%, at least 93%, at least 95%, at least 96%, at least 97%, at least 98%, at least 99%, or 100% identity to SEQ ID NO:5. For example, the first nucleic acid molecule can comprise a nucleotide sequence encoding the amino acid sequence having at least 90%, at least 93%, at least 95%, at least 96%, at least 97%, at least 98%, at least 99%, or 100% identity to one or more of SEQ ID NO:4, e.g., SEQ ID NO:5.

Recombinant expression of an antibody or fragment thereof described herein (e.g., a heavy or light chain of an antibody described herein) that specifically binds to involves construction of an expression vector containing a polynucleotide that encodes the antibody or fragment. Once a polynucleotide encoding an antibody or fragment thereof (e.g., heavy or light chain variable domains) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, e.g., in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, e.g., include the nucleotide sequence encoding the constant region of the antibody molecule (see, e.g., WO86/05807 and WO89/01036; and U.S. Pat. No. 5,122,464) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein.

A variety of host-expression vector systems can be utilized to express antibody molecules described. Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NSO, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, cells for expressing antibodies described herein (e.g., an antibody comprising the CDRs of any one of antibodies pab1949 or pab2044) are CHO cells, e.g., CHO cells from the CHO GS System™ (Lonza). In some embodiments, cells for expressing antibodies described herein are human cells, e.g., human cell lines. In some embodiments, a mammalian expression vector is pOptiVEC™ or pcDNA3.3. In some embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In certain embodiments, antibodies described herein are produced by CHO cells or NSO cells. In some embodiments, the expression of nucleotide sequences encoding antibodies described herein is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the antibody coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan J & Shenk T (1984) PNAS 81: 3655-3659). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol 153:516-544).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, recombinant proteins described herein (e.g., an antibody comprising the CDRs are produced in mammalian cells, such as CHO cells.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an antibody described herein or a fragment thereof. Such engineered cell lines can be particularly useful in screening and evaluation of compositions that interact directly or indirectly with the antibody molecule.

A number of selection systems can be used, including but not limited to, the herpes simplex virus thymidine kinase (Wigler M et al., (1977) Cell 11(1): 223-232), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-823) genes can be employed in tk-, hgprt- or aprt-cells, respectively. Also, antimetabolite resistance can be used as the basis of selection for the following genes: dhfr, which confers resistance to methotrexate (Wigler M et al., (1980) PNAS 77(6): 3567-3570; O'Hare K et al., (1981) PNAS 78: 1527-1531); gpt, which confers resistance to mycophenolic acid (Mulligan R C & Berg P (1981) PNAS 78(4): 2072-2076); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-215); and hygro, which confers resistance to hygromycin (Santerre R F et al., (1984) Gene 30(1-3): 147-156).

Once an antibody molecule described herein has been produced by recombinant expression, it can be purified by any method known in the art for purification of an immunoglobulin molecule, e.g., by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. Generally, an isolated antibody is one that is substantially free of other antibodies with different antigenic specificities than the isolated antibody. For example, in some embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, e.g., different post-translational modified forms of an antibody. When the antibody or fragment is recombinantly produced, it is also generally substantially free of culture medium, i.e., culture medium represents less than about 20%, 10%, 2%, 1%, 0.5%, or 0.1% of the volume of the protein preparation. When the antibody or fragment is produced by chemical synthesis, it is generally substantially free of chemical precursors or other chemicals, i.e., it is separated from chemical precursors or other chemicals which are involved in the synthesis of the protein. Accordingly, such preparations of the antibody or fragment have less than about 30%, 20%, 10%, or 5% (by dry weight) of chemical precursors or compounds other than the antibody or fragment of interest. In some embodiments, antibodies described herein are isolated or purified.

### Antibody Production

Still other aspects provide methods of preparing the recombinant protein, the methods including (a) culturing the cells; and (b) recovering the recombinant protein from the cultured cells. The cells can be cultured in various media. A commercially available medium can be used as a culture medium without limitation. All other essential supplements known to those skilled in the art can also be included at appropriate concentrations. Culture conditions, e.g., temperature, pH, etc., are those previously used together with the host cell selected for expression, and will be apparent to those skilled in the art. The recovering of the recombinant proteins can be performed by removing impurities by, e.g., centrifugation or ultrafiltration, and purifying the resultant by, e.g., affinity chromatography, etc. Other additional purification techniques, e.g., anion or cation exchange chromatography, hydrophobic interaction chromatography, hydroxylapatite chromatography, etc. can be used.

Recombinant proteins disclosed herein can be produced by any method known in the art for the synthesis of antibodies, e.g., by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, e.g., in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some embodiments, the recombinant proteins described herein are antibodies (e.g., recombinant antibodies) prepared, expressed, created, or isolated by any means that involves creation, e.g., via synthesis or genetic engineering of DNA sequences. In certain embodiments, such antibodies comprise sequences (e.g., DNA sequences or amino acid sequences) that do not naturally exist within the antibody germline repertoire of an animal or mammal (e.g., human) in vivo.

In some aspects, provided herein are methods of making recombinant proteins disclosed herein comprising culturing a cell or host cell as described herein. In some aspects, provided herein are methods of making a recombinant protein comprising expressing (e.g., recombinantly expressing) the antibodies using a cell or host cell described herein (e.g., a cell or a host cell comprising polynucleotides encoding an antibody described herein). In some embodiments, the cell is an isolated cell. In some embodiments, the exogenous polynucleotides has been introduced into the cell. In some embodiments, the method further comprises purifying the antibody obtained from the cell or host cell.

Antibodies can be prepared using a wide variety of techniques known in the art including the use of hybridoma, recombinant, and phage display technologies, or a combination thereof. For example, monoclonal antibodies can be produced using hybridoma techniques including those known in the art and taught, e.g., in Harlow E & Lane D, Antibodies: A Laboratory Manual (Cold Spring Harbor Laboratory Press, 2nd ed. 1988); Hammerling G J et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody described herein.

A “monoclonal antibody,” as used herein, is an antibody produced by a single cell (e.g., hybridoma or host cell producing a recombinant antibody), wherein the antibody immunospecifically binds to an antigen (e.g., human serum albumin) as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody can be a Fab fragment or a F(ab′)2 fragment. Monoclonal antibodies described herein can, e.g., be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies expressed thereby are well known in the art (see, e.g., Chapter 11 in: Short Protocols in Molecular Biology, (2002) 5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the antigen (e.g., human serum albumin)) used for immunization. Alternatively, lymphocytes can be immunized in vitro. Lymphocytes then are fused with myeloma cells using a suitable fusing agent, such as polyethylene glycol, to form a hybridoma cell. Some aspects of the disclosure provide a method of preparing the recombinant fusion protein, the method including: (a) culturing the cell; and (b) recovering the recombinant fusion protein from the cultured cell. The cell can be cultured in various media. For use as a culture medium, any commercially available medium can be used without limitation. All other necessary supplements known to those skilled in the art can also be included in suitable concentrations. Culture conditions, such as temperature, pH, and the like, have already been applied to host cells selected for expression, and will be apparent to those skilled in the art. The recombinant fusion protein can be purified by, for example, centrifugation or ultrafiltration to remove impurities, and the resultant product can be purified by using, for example, affinity chromatography or the like. Other purification techniques, such as anion or cation exchange chromatography, hydrophobic interaction chromatography, hydroxyapatite chromatography, and the like, can be used.

### Compositions and Uses Thereof

Some aspects of the disclosure provide a long-acting hFSH formulation including the recombinant fusion protein as an active ingredient.

Since the long-acting hFSH formulation uses the aforementioned recombinant fusion protein as an active ingredient, descriptions of common contents between the two will be omitted.

The recombinant fusion protein, which is an active ingredient of the preparation, can extend in vivo half-life of the recombinant protein through fusion with the antigen-binding fragment that binds to serum albumin, and accordingly, the therapeutic efficacy of hFSH can be improved.

In some embodiments, follitropin alfa (trade name: Gonal-F), which is generally used for infertility treatment, has a half-life of about 24 hours in the human body, and thus there is a limitation that it must be administered frequently to patients. However, the recombinant fusion according to some aspects of the disclosure is confirmed not only to exhibit an approximately 3-fold extended half-life in rats compared to the follitropin alfa, and as a result of measuring and comparing the ovarian volume under similar experimental conditions, but also to exhibit an ovarian growth promoting effect that is about 3 times higher than that of Gonal-F. Therefore, the long-acting hFSH preparation can be used for assisted reproductive technology for infertility treatment, such as in vitro fertilization-embryo transfer (IVF-ET), gamete intrafallopian injection (GIFT), zygote intrafallopian tube injection (ZIFT), and intracytoplasmic sperm injection (ICSI). Also, the long-acting hFSH preparation can be applied to IVF technology to induce superovulation in a subject, or can be used to treat anovulation, hypogonadism, and polycystic ovary syndrome.

In some embodiments, the long-acting hFSH preparation can be provided in the form of: a pharmaceutical composition for treatment of infertility, including: (a) the recombinant fusion protein in a pharmaceutically effective amount; and (b) a pharmaceutically acceptable carrier; a method of treating infertility by administering the pharmaceutical composition that includes the recombinant fusion protein; and a pharmaceutical use of the recombinant fusion protein for treatment of infertility.

The term “treatment” as used in the present specification refers to all activities that improve or beneficially change symptoms of infertility by administering the pharmaceutical composition.

The term “infertility”, a disease to be treated by the pharmaceutical composition, is a condition in which pregnancy does not happen even after being in a normal conjugal relationship for more than one year and refers to a condition that is treated or expected to be treated through administration of FSHs. The infertility can include both female infertility, including anovulation, and male infertility caused by sperm abnormalities.

The pharmaceutically acceptable carrier included in the pharmaceutical composition can be those conventionally used at the time of formulation, and examples thereof include lactose, dextrose, sucrose, sorbitol, mannitol, starch, acacia gum, calcium phosphate, alginate, gelatin, calcium silicate, microcrystalline cellulose, polyvinylpyrrolidone, water, syrup, methyl cellulose, methyl hydroxybenzoate, propyl hydroxybenzoate, talc, magnesium stearate, mineral oil, and the like, but embodiments are not particularly limited thereto. The pharmaceutical composition of the disclosure can further include, in addition to the aforementioned ingredients, a lubricant, a wetting agent, a sweetening agent, a flavoring agent, an emulsifier, a suspending agent, a preservative, and the like.

The pharmaceutical composition can be administered orally or parenterally. Preferably, the administration can be parenteral, and in the case of parenteral administration, the pharmaceutical composition can be administered via intravenous injection, subcutaneous injection, intramuscular injection, intraperitoneal injection, endothelial administration, topical administration, intranasal administration, intrapulmonary administration, intrarectal administration, and the like. In some embodiments, the pharmaceutical composition can be administered via subcutaneous or intramuscular injection.

The pharmaceutical composition can be administered in a pharmaceutically effective amount. The term “pharmaceutically effective amount” as used in the present specification refers to an amount sufficient to treat a disease with a reasonable benefit/risk ratio applicable to medical treatment, and an effective dose level can be determined according to factors, such as a disease type of a patient, severity, drug activity, drug sensitivity, an administration time, an administration rout and an excretion rate, a period of treatment, drugs being simultaneously used, and other factors well known in the medical field. The pharmaceutical compositions can be administered as an individual therapeutic agent or in combination with other therapeutic drugs, can be administered simultaneously with, separately from, or sequentially with conventional therapeutic drugs, and can be administered in a single dose or multiple doses. Considering all of the factors above, it is important to administer an amount that can achieve maximum effect with minimum amount without side effects, and such an amount can be easily determined by those skilled in the art. The term “pharmaceutically effective amount” as used in the present specification refers to an amount sufficient to treat a target disease or condition, and the amount can be changed depending on characteristics of a subject, an estradiol concentration, a cause of infertility, and a purpose of administration.

The pharmaceutical composition can be prepared in a unit dosage form by formulation using a pharmaceutically acceptable carrier and/or an excipient, or can be prepared in a multi-dose container, according to methods that can be easily carried out by those skilled in the art to which the disclosure pertains. Here, the dosage form can be a solution in an oily or aqueous medium, a suspension, an emulsion, an extract, a powder, a suppository, a powdered drug, a granule, a tablet, or a capsule, and can further include a dispersant or a stabilizer.

For example, the pharmaceutical composition can comprise (a) a pharmaceutically effective amount of the recombinant protein; and (b) a pharmaceutically acceptable carrier.

In some embodiments, the in vivo half-life of the pharmaceutical composition can exhibit a 2- to 20-fold increase, as compared with that of human FSH. The in vivo half-life can exhibit, e.g., about 2.5-fold to about 3.5-fold, about 3.5-fold to about 6-fold increase, about 4-fold to about 6-fold increase, about 4.5-fold to about 6-fold increase, about 5-fold to about 6-fold increase, about 5.5-fold to about 6-fold increase, about 3-fold to about 5.5-fold increase, about 3.5-fold to about 5.5-fold increase, about 4-fold to about 5.5-fold increase, about 4.5-fold to about 5.5-fold increase, about 5-fold to about 5.5-fold increase, about 3-fold to about 5-fold increase, about 3.5-fold to about 5-fold increase, about 4-fold to about 5-fold increase, about 4.5-fold to about 5-fold increase, about 3-fold to about 4.5-fold increase, about 3.5-fold to about 4.5-fold increase, about 4-fold to about 4.5-fold increase, or any fold or ranges of folds derived therefrom, as compared with that of human FSH. In some embodiments, the in vivo half-life of the human FSH can be evaluated after subcutaneous injection of the human FSH.

In some embodiments, the pharmaceutical composition can decrease white blood cell levels in blood. The white blood cells can be, e.g., neutrophils, monocytes, basophils, or a combination thereof. In some embodiments, the decreased white blood cell level can be sustained and maintained until day 20 after administration, until day 15 after administration, until day 12 after administration, until day 10 after administration, until day 8 after administration, until day 7 after administration, or any ranges derived therefrom.

The pharmaceutical composition can be prepared in a unit dosage form or in a multi-dose container by formulating using a pharmaceutically acceptable carrier and/or excipient according to a method that can be easily carried out by a person skilled in the art to which the present disclosure pertains. In this case, the formulation can be in the form of a solution, suspension, or emulsion in an oily or aqueous medium, or in the form of an extract, a suppository, a powder, granules, a tablet, or a capsule, and the formulation can further include a dispersing agent or a stabilizing agent.

Provided herein are compositions comprising a recombinant protein described herein having the desired degree of purity in a physiologically acceptable carrier, excipient or stabilizer (Remington's Pharmaceutical Sciences (1990) Mack Publishing Co., Easton, PA). Also disclosed herein are pharmaceutical compositions comprising a recombinant protein described herein and a pharmaceutically acceptable excipient. Acceptable carriers, excipients, or stabilizers are nontoxic to recipients at the dosages and concentrations employed.

The pharmaceutical composition for according to some aspects can be used after formulating in the form of oral preparations, such as powders, granules, tablets, capsules, suspensions, emulsions, syrups, aerosols, etc., external preparations, suppositories, or sterile injectable preparations according to common methods, and for formulation, the pharmaceutical composition can include an appropriate carrier, excipient, or diluent commonly used in the preparation of pharmaceutical compositions.

The carrier, excipient, or diluent can include various compounds or mixtures, such as lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, etc.

When formulated, it can be prepared using commonly used diluents or excipients, such as fillers, extenders, binders, wetting agents, disintegrants, surfactants, etc.

Solid formulations for oral administration can be prepared by mixing the recombinant fusion protein with at least one excipient, for example, starch, calcium carbonate, sucrose, lactose, gelatin, etc. In addition to simple excipients, lubricants such as magnesium stearate or talc can also be used.

Liquid formulations for oral administration can include suspensions, solutions for internal use, emulsions, syrups, etc. In addition to water and liquid paraffin, which are commonly used simple diluents, various excipients, for example, wetting agents, sweeteners, fragrances, preservatives, etc. can be included.

Formulations for parenteral administration include sterile aqueous solutions, non-aqueous solvents, suspensions, emulsions, lyophilized preparations, and suppositories. The non-aqueous solvents and suspensions can include propylene glycol, polyethylene glycol, vegetable oils such as olive oil, injectable esters such as ethyl oleate, etc. As a suppository base, witepsol, macrogol, tween 61, cacao butter, laurin butter, glycerol gelatin, etc. can be used.

Disclosed herein are methods of inducing superovulation of a subject in need thereof, comprising applying the pharmaceutical composition disclosed herein to in vitro fertilization-embryo transfer (IVF-ET), gamete intrafallopian transfer (GIFT), zygote intrafallopian transfer (ZIFT), intracytoplasmic sperm injection (ICSI), or in vitro fertilization. Disclosed herein are methods of treating anovulation, hypogonadism, or polycystic ovary syndrome in a subject in need thereof, comprising administering the pharmaceutical composition disclosed here to the subject.

Also disclosed herein are long-acting human follicle stimulating hormone (hFSH) compositions comprising the recombinant fusion protein disclosed herein as an active ingredient. In some embodiments, the long-acting hFSH composition is applied to in vitro fertilization-embryo transfer (IVF-ET), gamete intrafallopian transfer (GIFT), zygote intrafallopian transfer (ZIFT), intracytoplasmic sperm injection (ICSI), or in vitro fertilization, to induce superovulation of a subject. In some embodiments, the long-acting hFSH composition is used for treatment of anovulation, hypogonadism, or polycystic ovary syndrome.

As used herein, the term “treating” or “treatment” means all of actions by which symptoms of the disease or condition have improved, been eliminated, or been modified favorably by administering the compositions disclosed herein.

Also disclosed herein are uses of the compositions disclosed herein for inducing superovulation in subjects in need thereof. Also disclosed herein are the compositions disclosed herein for use in inducing superovulation in subjects in need thereof. Also disclosed herein are the use of the compositions disclosed herein for the manufacture of a medicament for inducing superovulation in subjects in need thereof.

Pharmaceutical compositions described herein can be useful in enhancing, inducing, or activating the activities of the recombinant proteins disclosed herein and treating a disease or condition.

The compositions to be used for in vivo administration can be sterile. This is readily accomplished by filtration through, e.g., sterile filtration membranes.

The long-acting hFSH composition can be applied to in vitro fertilization-embryo transfer (IVF-ET), gamete intrafallopian transfer (GIFT), zygote intrafallopian transfer (ZIFT), intracytoplasmic sperm injection (ICSI), or in vitro fertilization, to induce superovulation of a subject. In some embodiments, the long-acting hFSH composition is used for treatment of anovulation, hypogonadism, or polycystic ovary syndrome.

In some embodiments, the pharmaceutical composition can be a pharmaceutical composition for preventing or treating adult-onset still's disease, the pharmaceutical composition including the recombinant fusion protein as an active ingredient. Adult-onset still's disease is a multifactorial systemic autoinflammatory disease that has similar symptoms to systemic juvenile idiopathic arthritis, and is an inflammatory disease that occurs in adults, but the exact pathogenic mechanisms of the disease remain unknown. The adult-onset still's disease is characterized in that a concentration of FSH in the blood is increased and a concentration of FSH, which is an antagonist in vivo, is down-regulated. Meanwhile, it was reported that more than 50% of patients with adult-onset still's disease who received a recombinant FSH drug at doses of 80 mg/head and 160 mg/head, respectively, responded to the drug. In addition, clinical trials reported that the drug has a half-life of about 30 hours to about 40 hours in humans and is effective when administered three times a week. Thus, there is a problem in that the drug needs to be frequently administered, as a formulation for subcutaneous injection, to patients. In one exemplary embodiment, it was confirmed that the recombinant fusion protein exhibited a half-life about 3.5 times extended in rats, as compared with the recombinant FSH, and had the same and similar activity even when a small dose is administered. Therefore, the recombinant fusion protein can be effectively used for the treatment of adult-onset still's disease.

Disclosed herein are compositions comprising the recombinant fusion protein disclosed herein and a carrier. Disclosed herein are pharmaceutical compositions comprising the recombinant fusion protein disclosed herein and a pharmaceutically acceptable carrier. Disclosed herein are kits comprising the composition disclosed herein and a label comprising instructions for a use.

Disclosed herein are methods of inducing superovulation of a subject in need thereof, comprising applying the pharmaceutical composition disclosed herein to in vitro fertilization-embryo transfer (IVF-ET), gamete intrafallopian transfer (GIFT), zygote intrafallopian transfer (ZIFT), intracytoplasmic sperm injection (ICSI), or in vitro fertilization. Disclosed herein are methods of treating anovulation, hypogonadism, or polycystic ovary syndrome in a subject in need thereof, comprising administering the pharmaceutical composition disclosed here to the subject.

Also disclosed herein are long-acting human follicle stimulating hormone (hFSH) compositions comprising the recombinant fusion protein disclosed herein as an active ingredient. In some embodiments, the long-acting hFSH composition is applied to in vitro fertilization-embryo transfer (IVF-ET), gamete intrafallopian transfer (GIFT), zygote intrafallopian transfer (ZIFT), intracytoplasmic sperm injection (ICSI), or in vitro fertilization, to induce superovulation of a subject. In some embodiments, the long-acting hFSH composition is used for treatment of anovulation, hypogonadism, or polycystic ovary syndrome.

In some embodiments, the composition is administered subcutaneously or intramuscularly to the subject.

Still other aspects provide pharmaceutical compositions including the recombinant fusion protein as an active ingredient. Specific details of the recombinant fusion protein are as described above.

### Routes of Administration & Dosages

The pharmaceutical compositions of the present disclosure can be administered to a subject through a variety of administration routes including oral, transcutaneous, subcutaneous, intravenous, and intramuscular administration routes.

The amount of a recombinant protein or composition disclosed herein that will be effective in the treatment and/or prevention of a condition will depend on the nature of the disease and can be determined by standard clinical techniques.

In the present disclosure, the amount of the recombinant protein disclosed herein that is actually administered is determined in light of various relevant factors including the disease to be treated, a selected route of administration, the age, sex and body weight of a patient, and severity of the disease, and the type of a bioactive polypeptide as an active ingredient. Since the recombinant protein of the present disclosure has excellent sustainability in blood, the number and frequency of administration of the peptide preparations comprising the recombinant protein of the present disclosure can be noticeably reduced.

The pharmaceutical composition is administered in a pharmaceutically effective amount. As used herein, the “pharmaceutically effective amount” or “effective amount” in the context of the administration of a therapy to a subject refers to the amount of a therapy that achieves a desired prophylactic or therapeutic effect. An effective dose level can be determined depending on factors including a patient's disease type, severity, drug activity, drug sensitivity, administration time, administration route and excretion ratio, treatment period, and co-administered drugs, and other factors well known in the medical field. The pharmaceutical composition can be administered as a single therapeutic agent or in combination with other therapeutic drugs, and can be administered with existing therapeutic drugs simultaneously, separately, or sequentially, once or in a few divided doses. It is important to administer the composition in a minimum amount sufficient to obtain the maximum effect without any side effects, considering all the factors, and this amount can be easily determined by those skilled in the art. As used herein, the term “pharmaceutically effective amount” refers to an amount sufficient to treat or induce the disease or condition discussed herein, such as inducing superovulation or treating anovulation, hypogonadism, or polycystic ovary syndrome.

An appropriate dosage of the pharmaceutical composition varies depending on a patient's conditions, body weight, disease severity, drug formulation, administration route and period, but can be appropriately selected by those skilled in the art. However, for desirable effects, the pharmaceutical composition can be administered at a daily dose of 0.0001 mg/kg to 2,000 mg/kg, and specifically, 0.001 mg/kg to 2,000 mg/kg. Administration can be performed once a day, or in several divided doses.

The precise dose to be employed in a composition will also depend on the route of administration, and the seriousness of the disease, and should be decided according to the judgment of the practitioner and each subject's circumstances. For example, effective doses can also vary depending upon means of administration, target site, physiological state of the patient (including age, body weight and health), other medications administered, or whether treatment is prophylactic or therapeutic. Usually, the patient is a human but can be a non-human, such as pets, e.g., dogs and cats. Treatment dosages are optimally titrated to optimize safety and efficacy.

In certain embodiments, an in vitro assay is employed to help identify optimal dosage ranges. Effective doses can be extrapolated from dose response curves derived from in vitro or animal model test systems.

In some embodiments, the recombinant fusion protein can be administered at a dose of 0.001 mg/kg to 2,000 mg/kg. For example, the recombinant fusion protein can be administered at a dose of 0.001 mg/kg to 0.01 mg/kg, 0.1 mg/kg to 1 mg/kg, 1.5 mg/kg to 2 mg/kg, 4 mg/kg to 10 mg/kg, 15 mg/kg to 20 mg/kg, 30 mg/kg to 40 mg/kg, 60 mg/kg to 80 mg/kg, 100 mg/kg to 200 mg/kg, or any dose or ranges of doses derived therefrom.

The pharmaceutical composition for treating diseases or inducing conditions can be administered to mammals, such as rats, mice, livestock, humans, etc., via various routes. All modes of administration can be contemplated, for example, by oral, rectal or intravenous, intramuscular, subcutaneous, or intradural administration, or intracerebroventricular injection.

The pharmaceutical composition can be orally or parenterally administered. Specifically, the pharmaceutical composition can be parenterally administered, and in this case, it can be administered by intravenous injection, subcutaneous injection, intramuscular injection, intraperitoneal injection, endothelial administration, topical administration, intranasal administration, intrapulmonary administration, and rectal administration. In some embodiments, it can be administered in the form of subcutaneous injection. When orally administered, a protein or peptide is digested, and therefore, it is required to formulate an oral composition by coating the active ingredient or protecting it from degradation in the stomach. In addition, the pharmaceutical composition can be administered by any device capable of delivering an active substance to target cells.

Still other aspects provide a health functional food composition for preventing or improving conditions or diseases, the health functional food composition including the recombinant fusion protein as an active ingredient. Still other aspects provide a health functional food composition, the health functional food composition including the recombinant fusion protein as an active ingredient.

With regard to the health functional food composition, the recombinant fusion protein can be added as it is or can be used with other food or food ingredients, when the recombinant fusion protein is used as an additive for the health functional food and can be used appropriately according to a common method. A mixing amount of the active ingredient can be appropriately determined according to each purpose of use, such as prevention, health, treatment, etc.

The formulation of the health functional food can be in the form of powders, granules, pills, tablets, and capsules, as well as in the form of general foods or beverages.

The type of food is not particularly limited, and examples of the food, to which the substance can be added, can include meats, sausages, bread, chocolates, candies, snacks, confectionery, pizza, ramen, other noodles, gums, dairy products including ice cream, various soups, beverages, teas, drinks, alcoholic beverages, vitamin complexes, etc., and can include all foods in common sense.

In general, in the preparation of foods or beverages, the recombinant fusion protein can be added in an amount of 15 parts by weight or less, and specifically, 10 parts by weight or less, based on 100 parts by weight of the raw material. However, in the case of long-term intake for the purpose of health and hygiene or for the purpose of health control, the amount can be adjusted to be below the above range. Further, the present disclosure has no problem in terms of safety because a fraction from a natural product is used. Accordingly, the amount can be above the range.

Among the health functional foods, beverages can include various flavoring agents or natural carbohydrates as additional ingredients, like in common beverages. The above-mentioned natural carbohydrates can include monosaccharides such as glucose and fructose, disaccharides such as maltose and sucrose, polysaccharides such as dextrin and cyclodextrin, and sugar alcohols such as xylitol, sorbitol, erythritol, etc. As a sweetener, natural sweeteners such as taumatin and stevia extract, synthetic sweeteners such as saccharin and aspartame, etc. can be used. A proportion of the natural carbohydrate can be about 0.01 g to 0.04 g, and specifically, about 0.02 g to 0.03 g per 100 mL of the beverage according to the present disclosure.

In addition, the health functional food composition according to some aspects can include various nutrients, vitamins, electrolytes, flavoring agents, coloring agents, pectic acid and salts thereof, alginic acid and salts thereof, organic acids, protective colloidal thickeners, pH adjusters, stabilizers, preservatives, glycerin, alcohols, and carbonating agents used in carbonated beverages. In addition, the composition of the present disclosure can include fruit flesh for the preparation of natural fruit juice, fruit juice beverages, and vegetable beverages. These components can be used independently or in a mixture. A proportion of these additives is not limited but is generally selected from the range of 0.01 part by weight to 0.1 part by weight, relative to 100 parts by weight of the health functional food composition.

As described above, the recombinant fusion protein can exhibit, e.g., a half-life about 3.5 times extended in rats, as compared with the human recombinant FSH, and exhibits a biological activity at a similar level to that of FSH not fused to SL335. Therefore, since the recombinant fusion protein can exhibit similar efficacy even with less frequency of administration, patients can be administered with the drug at more convenient intervals.

### Kits

Provided herein are kits comprising one or more recombinant proteins described herein or conjugates thereof. Disclosed herein are kits comprising the compositions disclosed herein and labels comprising instructions for uses thereof. In some embodiments, provided herein is a pharmaceutical pack or kit comprising one or more containers filled with one or more of the ingredients of the pharmaceutical compositions described herein, such as one or more recombinant proteins provided herein. In some embodiments, the kits contain a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration. Also provided herein are kits that can be used in the above methods. In some embodiments, a kit comprises a recombinant protein described herein, e.g., a purified recombinant protein, in one or more containers. In some embodiments, kits described herein contain a substantially isolated antigen(s) (e.g., human serum albumin) that can be used as a control. In other embodiments, the kits described herein further comprise a control antibody which does not react with a serum albumin antigen. In other embodiments, kits described herein contain one or more elements for detecting the binding of a recombinant protein to a serum albumin antigen (e.g., the recombinant protein can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized serum albumin antigen. The serum albumin antigen provided in the kit can also be attached to a solid support. In some embodiments, the detecting means of the above-described kits include a solid support to which a serum albumin antigen is attached. Such kits can also include a non-attached reporter-labeled anti-human antibody or anti-mouse/rat antibody. In binding of the antibody to the serum albumin, the antigen can be detected by binding of the said reporter-labeled antibody.

Reference will now be made in detail to embodiments, examples of which are illustrated in the accompanying drawings, wherein like reference numerals refer to like elements throughout. In this regard, the present embodiments can have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the disclosure will be described in detail with reference to Examples below. However, these Examples are for illustrative purposes only, and the scope of the disclosure is not intended to be limited by these Examples.

### Examples

### Example 1. Preparation of Recombinant Fusion Protein Including Follicle Stimulating Hormone and Antigen-Binding Fragment to Serum Albumin

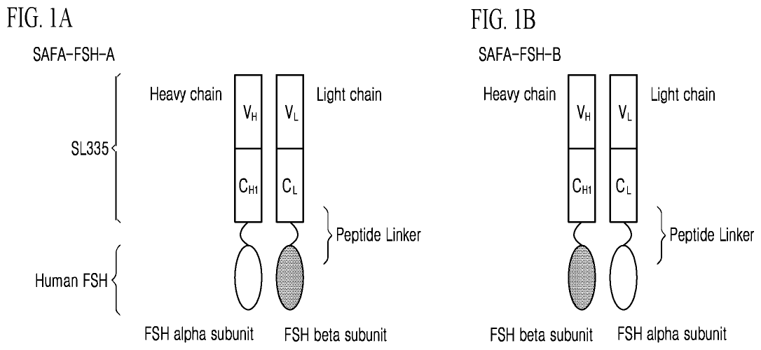

In this example, a recombinant fusion protein including a follicle stimulating hormone (FSH) and an antigen-binding fragment to serum albumin was prepared. In recombinant fusion protein as shown in FIG. 1, a human FSH beta subunit and a human FSH alpha subunit were each linked to the C-terminal region of ‘SL335’, which is a human anti-serum albumin Fab antibody fragment, via peptide linkers. By modifying a combination of heavy chain fragments or light chain fragments to which the human FSH alpha/beta subunits were linked, recombinant fusion proteins, i.e., SAFA-FSH-A and SAFA-FSH-B proteins, fusion proteins were prepared. Specifically, in this example, i) a SAFA-FSH-A protein comprising of: a heavy chain composed of [SL335 heavy chain fragment of SEQ ID NO:6-linker of SEQ ID NO:88-FSH alpha subunit of SEQ ID NO:1]; and a light chain composed of [SL335 light chain fragment of SEQ ID NO:14-linker of SEQ ID NO:85-FSH beta subunit of SEQ ID NO:4], and ii) a SAFA-FSH-B protein comprising of: a heavy chain composed of [SL335 heavy chain fragment of SEQ ID NO:6-linker of SEQ ID NO:85-FSH beta subunit of SEQ ID NO:4]; and a light chain composed of [SL335 light chain fragment of SEQ ID NO:14-linker of SEQ ID NO:88-FSH alpha subunit of SEQ ID NO:1] were each prepared.

### 1-1. Preparation of Expression Vector for Preparation of Recombinant Fusion Protein.

Genes used in an expression vector according to some embodiments were subjected to codon optimization by Cosmogenetech Co., Ltd. (South Korea), the genetic information of the light chain fragments and/or heavy chain fragments of the Fab antibody (SL335) that binds to serum albumin was the same as SEQ ID NOS:7, 8, 15, and 16, the genetic information of the peptide linker was the same as SEQ ID NOS:86, 87, 89, 90, and the genetic information of the FSH alpha subunit and the FSH beta subunit genes was the same as the FSH alpha subunit (by UniProKB P01215) and the FSH beta subunit (by UniProKB P01225) (SEQ ID NOS:2, 3, 5, and 9).

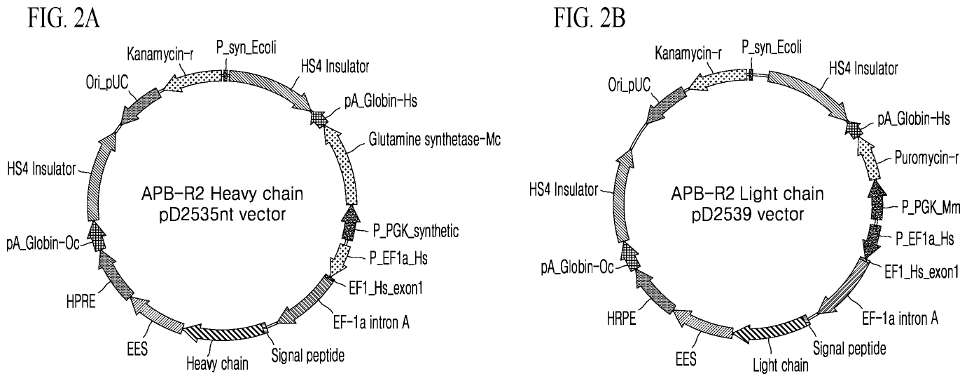

The recombinant genes of the SAFA-FSH heavy chain, i.e., the SL335H-linker-FSH alpha and the SL335H-linker-FSH beta (SEQ ID NOS:20 and 96), prepared by the gene synthesis and a pD2535NT (ATUM) expression vector were each treated with a BbsI (Takara, Japan) restriction enzyme to cleave a BbsI site, and then were each treated with a T4 DNA ligase (Takara, Japan) to be linked into the expression vector. In addition, the recombinant genes of the SAFA-FSH light chain, i.e., the SL335L-linker-FSH beta and the SL335L-linker-FSH alpha (SEQ ID NOS:94 and 98), prepared by the gene synthesis and a pD2359 (ATUM) expression vector were each treated with BsrGI (NEB, USA) and BbsI (Takara, Japan) restriction enzymes to cleave BsrGI and BbsI sites, and then were each treated with a T4 DNA ligase (Takara, Japan) to be linked into the expression vector. Specifically, the expression vectors for the preparation of the SAFA-FSH-A and SAFA-FSH-B proteins according to an embodiment are as shown in FIG. 2.

### 1-2. Preparation of Transient Expression Cells.

After adding ExpiCHO-S™ cells (ThermoFhisher scientific) to a 125 ml culture flask containing an expression medium (ExpiCHO expression media, Thermo Fisher Scientific), the cells were cultured in a shaking incubator under conditions of a temperature of 37° C., 140 rpm, 5% CO₂, and 80% humidity. Afterwards, for the preparation of transient expression cells, the cultured cells were seeded into the culture at a concentration of 6.0×10⁶ cells/ml, and then transfected with plasmid vectors (pD2535NT and pD2539) to which each of the heavy chain genes and the light chain genes prepared in Example 1-1 were inserted. Then, the transfected cells were cultured for 16 hours in a shaking incubator under the same conditions as described above and were subsequently treated with ExpiCHO feed and an enhancer. On Day 3 of the culture, the ExpiCHO feed was additionally treated, the incubator temperature was set to 37° C., and the cells were cultured for 8 days. After completion of the culture, the culture medium thus obtained was subjected to centrifugation under conditions of 4,000 rpm, 15 minutes, and 4° C., so as to separate the cells from the culture medium. Next, the separated culture medium was passed through a 0.2 m filter paper to remove impurities.

Meanwhile, as a result of comparing and confirming the expression patterns and productivity of the recombinant fusion proteins, i.e., the SAFA-FSH-A and SAFA-FSH-B proteins, including the FSH and the antigen-binding fragment to serum albumin, it was confirmed that the SAFA-FSH-B protein had a relatively stable structure and showed excellent productivity (not shown). Therefore, in the following, an experiment was performed by selecting the SAFA-FSH-B protein as the recombinant fusion protein according to an embodiment.

### 1-3. Preparation of Stabilized Cell Lines

A stabilized cell line was prepared by using HD-BIOP3 GS null CHO-K1 cells (Horizon Discovery). Specifically, cells were seeded at a concentration of 3.0×10⁵ cells/ml into a CD FortiCHO (Thermo Fisher Scientific) medium supplemented with 4 mM of L-glutamine and were subjected to seed culture in a shaking incubator under conditions of a temperature of 37° C., 5% CO₂, and humidity of 80% or higher. For transfection, the cultured cells were seeded at a concentration of 1.0×10⁶ cells/ml, and the SAFA-FSH-B plasmid vectors (pD2535NT and pD2539) prepared in Example 1-1 were each added to an OptiPRO SFM medium together with a Freestyle max reagent (Invitrogen, Carlsbad, California). After the seeded cells were transfected with the vector, the transfected cells were cultured for 2 days under conditions of a temperature of 37° C., 5% CO₂, and humidity of 80% or higher. Afterwards, to proceed with stable pool selection, the medium was replaced with L-glutamine-free CD FortiCHO medium by centrifugation, and the cells were treated with 50 μM methionine sulfoximine (MSX) (Sigma-Aldrich, St. St. Louis, Missouri) and 10 μg/ml of puromycin (Thermo Fisher Scientific) every two days to remove cells that were not injected into the vector. Thereafter, by using a centrifuge, the medium was replaced with a CD FortiCHO medium containing both MSX and puromycin at intervals of 7 to 10 days, and the cells were cultured for 21 days while maintaining the number of cells at 5.0×10⁵ cells/ml each time. Afterwards, when the viability was recovered to 90% or more, a stock with a concentration of 1.0×10⁷ cells/ml was prepared.

### 1-4. Separation and Purification of SAFA-FSH Protein.

The protein samples present in the culture medium of Example 1-3 were purified by sequentially performing affinity chromatography (AC), multimodal anion exchange chromatography (MAEX), and cation exchange chromatography (CEX). Specifically, AC was performed by using CaptureSelect CH1-XL resins (Thermo Fisher, USA) to bind the culture medium to the resins at a flow rate of 10 ml/min, and then, the recombinant fusion protein was recovered by a pH elution method. The AC-purified protein was subjected to MAEX by using Capo Adhere ImpRes resins (Cytiva, Sweden) at a flow rate of 5 ml/min, and then, the recombinant fusion protein was recovered by an FT mode method. Finally, CEX was performed by using CM Sepharose® Fast Flow resins at a flow rate of 5 ml/min. Through these separation and purification methods, the SAFA-FSH-B protein was obtained.

### Example 2. Confirmation of Physical Properties of SAFA-FSH Protein

### 2-1. Size Analysis

To confirm the size of the recombinant fusion protein prepared in Example 1, sodium dodecyl sulfate polyacrylamide gel electrophoresis (SDS-PAGE) was performed thereon. Specifically, protein samples were prepared under reducing conditions and non-reducing conditions by using a non-reducing (NR) 4×SDS sample buffer (Thermo Fisher Scientific). In the case of reducing conditions, samples heated at 100° C. for 5 minutes and samples not heated were prepared together to compare the shape and size of proteins depending on heat. To compare the size of proteins in each condition, a protein size marker (SMOBio, Taiwan) was prepared. The prepared protein samples were each titrated to a 4-15% Mini-protein TGX precast gel (15-well, by Bio-Rad) to be contained in 1.175 g or 2.35 g per well, and then subjected to electrophoresis in a tris-glycine SDS running buffer at 150 voltage (V) for 50 minutes. After completion of the electrophoresis, the SDS-PAGE gel was stained with EZ-Gel staining solution (DoGenBio, South Korea) for 1 hour, followed by destaining in distilled water for 1 day.

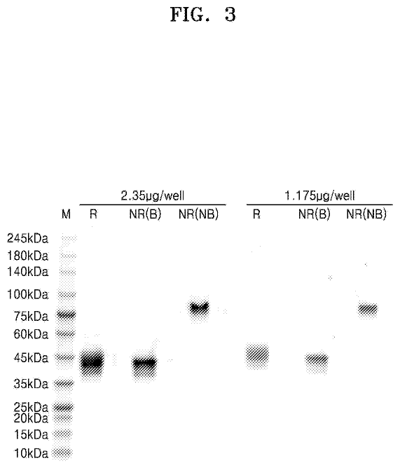

As a result, as shown in FIG. 3, under the reducing condition (R) and the non-reducing condition (NR (B)) in which heat was applied for 5 minutes, the heavy chain protein and the light chain protein underwent additional N-linked glycosylation so that the heavy chain bands were detected at about 40 kDa position which is higher than the theoretical size of 34.109 kDa, and the light chain bands were detected at 40 kDa to 45 kDa positions, which is higher than the theoretical size of 37.081 kDa. Meanwhile, under the non-reducing condition (NR(NB)) with no heat applied, the protein bands corresponding to the intact form of SAFA-FSH with the heavy chain and the light chain combined were observed at about 80 kDA position which is higher than the theoretical size of 71.19 kDa (heavy chain: 34.109 kDa, and light chain: 37.081 kDa).

### 2-2. Purity Assay

To measure the purity of the recombinant fusion protein according to an embodiment as prepared in Example 1, SE-HPLC was performed thereon. First, HPLC equipment with a TSKgel G3000SWXL 7.8×300 mm (Tosoh Bioscience, Japan) column and an Alliance HPLC system (Waters, Milford, MA) was subjected to equilibration by using a 200 mM sodium phosphate pH 7.0 buffer containing 100 mM NaCl. A sample to be analyzed was prepared by dilution with 200 mM sodium phosphate, 100 mM NaCl, and a pH 7.0 buffer, and 50 g of the prepared sample was loaded onto the column. The SE-HPLC analysis was performed for 40 minutes under conditions of a flow rate of 0.5 ml/min and a maximum pressure of 1,000 psi, and the purity was measured at a wavelength of A280 nm.

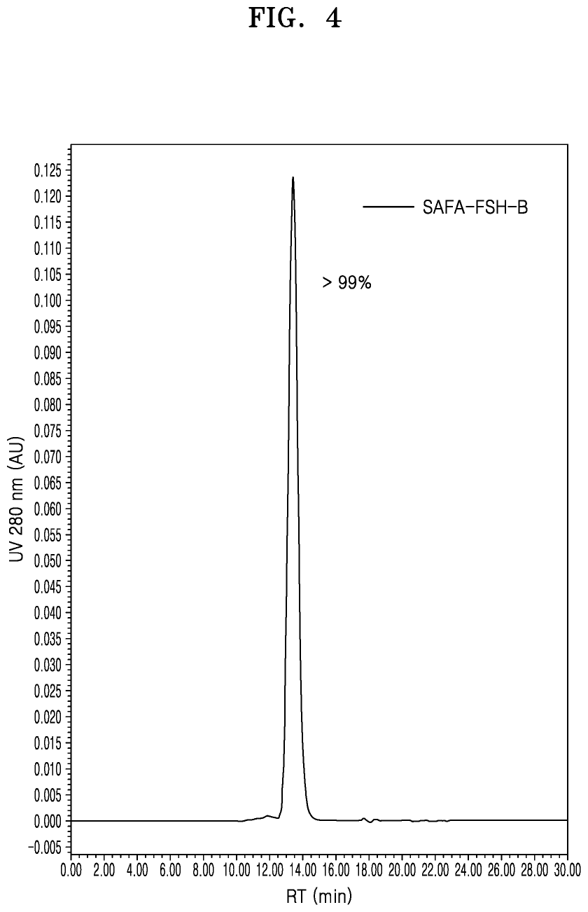

As a result, as shown in FIG. 4, it was confirmed that the SAFA-FSH protein had a purity of 99% or more.

### 2-3. Analysis of Isoelectric Point

To measure the isoelectric point of the recombinant fusion protein according to an embodiment as prepared in Example 1, isoelectric focusing analysis (IEF) was performed thereon. Specifically, analysis using pH 3-10 IEF gel (Invitrogen™ Novex™ pH 3-10 IEF protein gels, ThermoFisher Scientific, USA) and capillary isoelectric focusing (cIEF) analysis using PA800 (SCIEX, USA) equipment were performed. The pH 3-10 IEF gel was loaded with 5 μg of the sample and proceeded under conditions of 100 V for 1 hour, 200 V for 1 hour, and 500 V for 1 hour. The sample was fixed with 12% trichloroacetic acid (TCA), stained with coomassie brilliant blue (CBB), and then, analyzed for protein pI by using ChemiDoc MP Imagers (Bio-Rad). In addition, for the cIEF analysis, by using Neutral Capillary, 50 m id×45 cm and cIEF gel (Polymer solution) (Beckman Coulter, Inc.), 10 μL of 10 mg/ml SAFA-FSH and cIEF peptide marker (pI 10, 7.0, 5.5, 4.1, Beckman Coulter, Inc.) were mixed, followed by titration to neutral capillary, so as to analyze the isoelectric point of the SAFA-FSH protein by using a 32 Karat software.

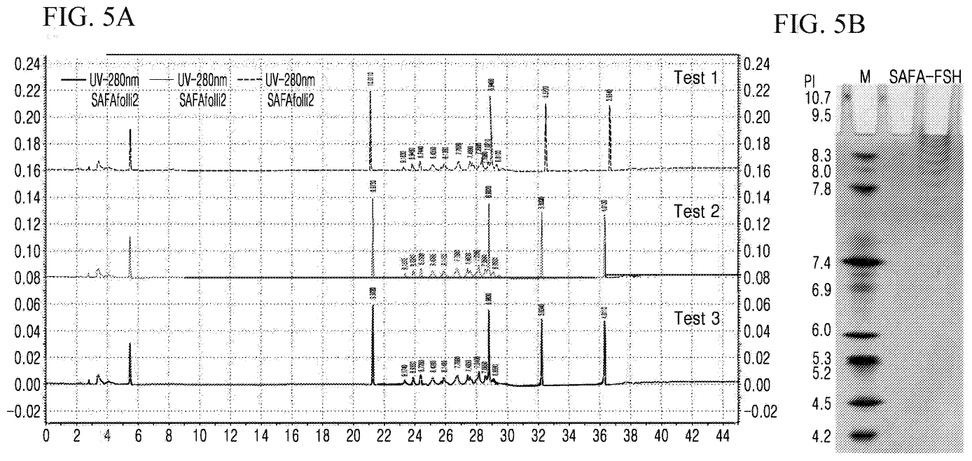

As a result, as shown in FIG. 5, bands and peaks were observed between pI 6.5 and pl 9.2, and the bands and peaks appeared due to N-linked glycans between the FSH alpha subunit and the FSH beta subunit.

### 2-4. Analysis of Molecular Weight of Light Chain and Heavy Chain

To measure the molecular weight of the recombinant fusion protein according to an embodiment as prepared in Example 1, capillary electrophoresis and intact mass spectrometry analysis were performed thereon. To confirm changes in the protein size caused by N-linked glycans included in the FSH alpha subunit and the FSH beta subunit, PNGase F (NEB, USA), which cleaves the N-linked glycans, was treated under the reducing condition, and then, SDS-PAGE and capillary electrophoresis were used to confirm changes in molecular weight.

For SDS-PAGE, the SAFA-FSH sample that has undergone the PNGase F enzymatic reaction was titrated to a 4-15% Mini-protein TGX precast gel (15-well, Bio-Rad) to be contained in 2 g per well, and then subjected to electrophoresis in a tris-glycine SDS running buffer at 150 V for 50 minutes. After completion of the electrophoresis, the SDS-PAGE gel was stained with EZ-Gel staining solution (DoGenBio, South Korea) for 1 hour, followed by destaining in distilled water for 1 day, so as to confirm the cleavage of the N-linked glycans by the PNGase F enzymatic treatment.

The sample not treated with the PNGase F enzyme and the sample in which N-linked glycans were cleaved by the PNGase F enzyme treatment were subjected to bare fused-silica capillary (50 m ID×57 cm, Beckman Coulter) and SDS-MW size-standard (10 kDa to 225 kDa, Beckman Coulter) by using PA800 equipment, so as to confirm the size and molecular weight of the protein by using a 32 Karat software.

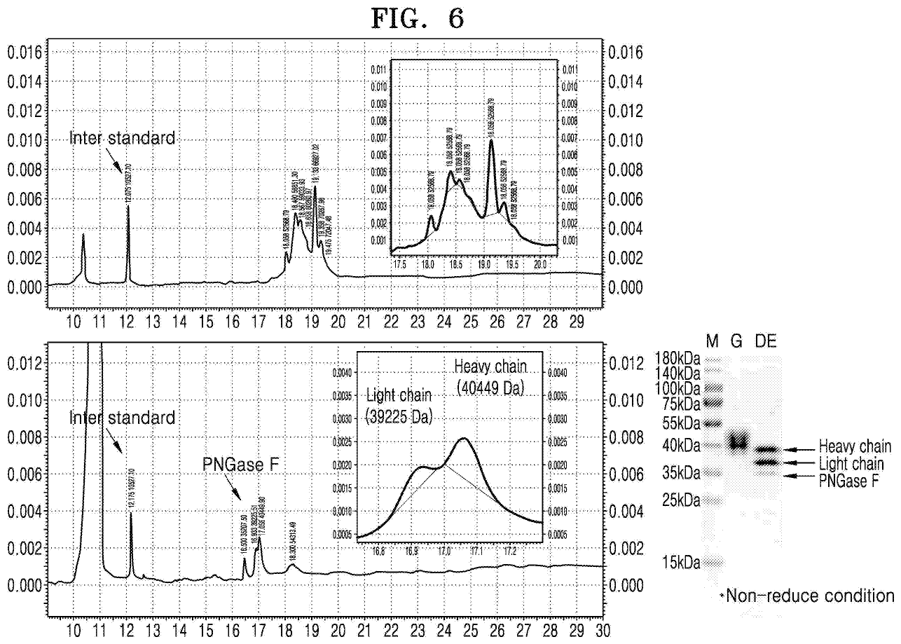

As a result, as shown in Table 1 and FIG. 6, it was confirmed that the mass of the light chain (SL335L+FSH alpha) of the SAFA-FSH protein was about 39.22 kDa, and the mass of the heavy chain (SL335H-FSH beta) of the protein was about 40.45 kDa.

TABLE 1

| | Theoretical | Migration | Molecular |
|---|---|---|---|
| | MW | time | weight |
| Protein | (kDa) | (min) | (kDa) |
| Inter standard | 10 | 12.175 | 10.527 |
| SL335L + FSH alpha | 34.109 | 16.933 | 39.225 |
| PNGase F | 36 | 16.500 | 35.207 |
| SL335H + FSH beta | 37.081 | 17.058 | 40.449 |

In addition, intact mass spectrometry analysis was carried out under reducing conditions (20 mM DTT, 37° C.) and measured by using ACQUTY I-Class UPLC (Waters) and SYNAPT G2-SI MS/MS system (Waters). The results were the analyzed by using a Flexanalysis Flexcontrol Software Version 3.0.

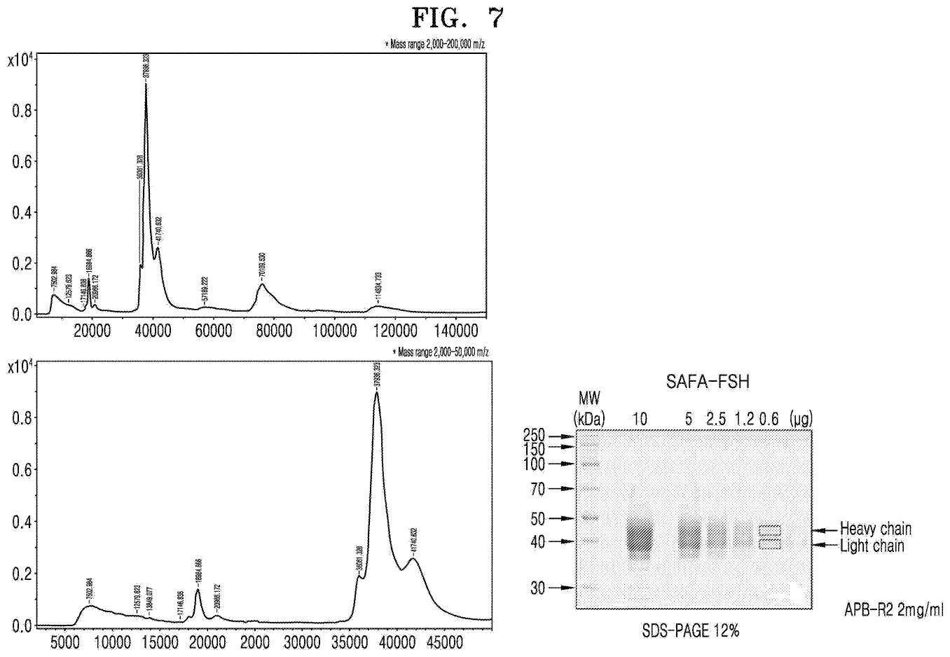

As a result, as shown in Table 2 and FIG. 7, it was confirmed that the mass of the light chain (SL335L+FSH alpha) of the SAFA-FSH protein was about 37.94 kDa, and the mass of the heavy chain (SL335H-FSH beta) of the protein was about 41.74 kDa.

TABLE 2

| | Theoretical | Molecular |
|---|---|---|
| | MW | weight |
| Protein | (kDa) | (Da) |
| SAFA-FSH | 71.173 | 76109.536 |
| SL335L + FSH alpha | 34.109 | 37936.323 |
| SL335H + FSH beta | 37.081 | 41740.632 |

### 2-5. Sequencing

For sequencing of the recombinant fusion protein according to an embodiment as prepared in Example 1, the N-terminal sequencing and peptide mapping analysis of the heavy chain and the light chain of the protein were performed. Specifically, the N-terminal sequencing and the peptide mapping analysis of the heavy chain and the light chain of the SAFA-FSH protein were performed by using Dionex UHPLC (Thermo Fisher Scientific) and Q-TOF 5600+MS/MS system (AB SCIEX, CA, USA). Acquity UPLC® BEH130 C4, 1.7 μm column was used for the measurement using mobile phase acetonitrile (ACN; JTBaker) and at a flow rate of 0.3 ml/min.

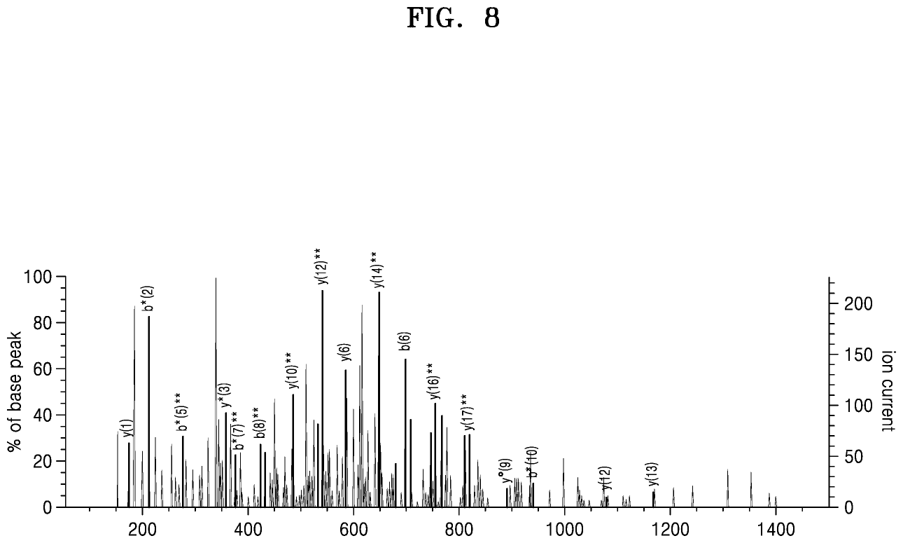

As a result, as shown in FIG. 8, the N-terminal sequences of the heavy chain and the light chain of the SAFA-FSH protein were confirmed 100%.

### Example 3. Confirmation of Biological Properties of SAFA-FSH Protein

### 3-1. Analysis of Binding Ability for Human Serum Albumin

By using Bio-Layer Interferometry (BLI) and Octet® BLI systems (Octet® Red, Sartorius, Germany), the binding ability of the SAFA-FSH protein to human serum albumin was analyzed. On an AR2G biosensor chip (Sartorius, Germany), human serum albumin (Sigma) was immobilized at a concentration of 30 μg/mL with a 10 mM acetate buffer pH 5.0, and the SAFA-FSH protein was added thereto at concentrations of 6.25 nM, 3.125 nM, 1.56 nM, and 0.78 nM (association: 600 s, dissociation: 900 s), so as to confirm the binding ability between the two under conditions of pH 7.4 or pH 6.0 conditions.

TABLE 3

| | KD | kon | kdis | Full | Full |
|---|---|---|---|---|---|
| pH | (M) | (1/Ms) | (1/s) | X\hat{ }2 | R\hat{ }2 |
| 7.4 | 3.59E−10 | 3.79E+05 | 1.36E−04 | 0.950722 | 0.998298 |
| 6.0 | 3.13E−10 | 4.75E+05 | 1.49E−04 | 2.225859 | 0.996373 |

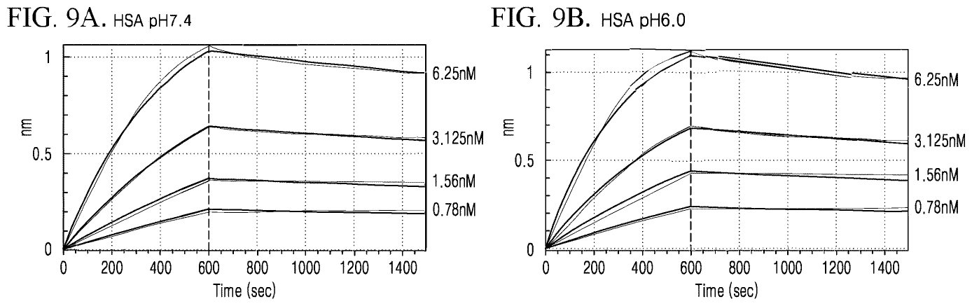

As a result, as shown in Table 3 and FIG. 9, it was confirmed that the SAFA-FSH protein and human serum albumin were able to form an effective bond at pH 7.4 or pH 6.0.

### 3-2. Confirmation of Binding to FSH Receptor

By using a human FSHR stable cell line (HEK293, Creative Biogene, USA) expressing human FSH receptors on the surface, the binding between the SAFA-FSH protein and the FSH receptors was analyzed with a CytoFLEX flow cytometer (Beckman Coulter, USA).

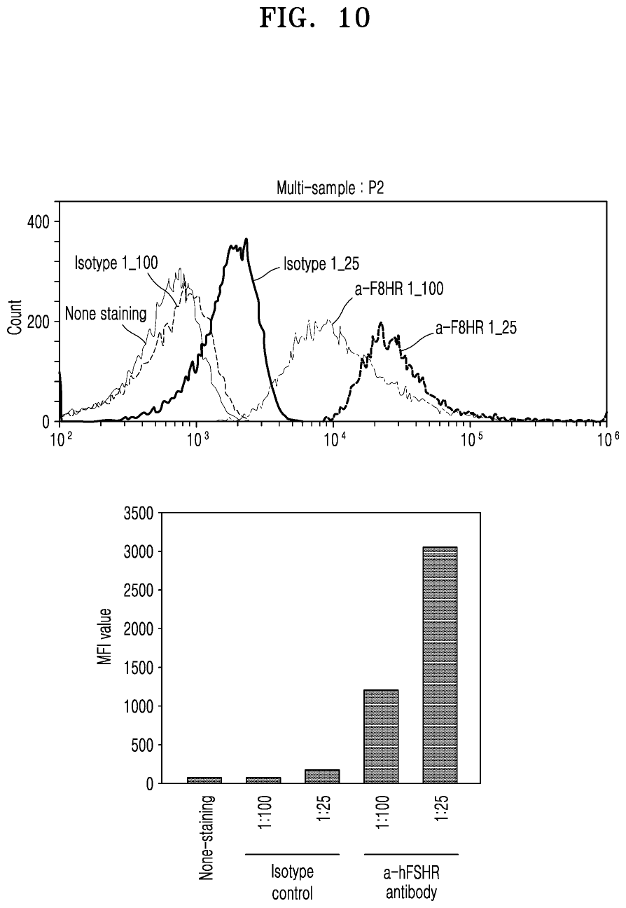

First, the human FSHR stable cells were dissolved in MACS buffer (PBS containing 0.5% BSA and 2 mM EDTA) at a concentration of 1×10⁶ cells/ml, and then added in an amount of 100 μl (1.0×10⁵ cells/test) to a 1.7 ml tube. The cells were then centrifuged at 500×g at 4° C. for 5 minutes. After removing the supernatant from the 1.7 ml tube, 100 μl of each of human FSHR APC-conjugated antibody (R&D systems, USA) and APC mouse IgG2a isotype control (R&D systems, USA) that were diluted with MACS buffer at a ratio of 1:25 and 1:100 was added to the same tube to proceed a reaction at 4° C. for 30 minutes. After 500 l of MACS buffer was added to the tube and the cells were centrifuged at 500×g at 4° C. for 5 minutes, the supernatant was removed therefrom to remove unbound antibodies. 200 μl of 0.4% PFA was added to the tube, and then the cells were loosened and fixed by using a pipette. By measuring the APC sensitivity with a CytoFLEX device, the expression of human FSHR was confirmed. As a result, as shown in FIG. 10, it was confirmed that the human FSH receptors resent on the surface of the human FSHR stable cells were expressed at a high level.

Subsequently, the binding between the SAFA-FSH protein and the human FSHR was confirmed by using the human FSHR stable cell line in which the expression of human FSHR was confirmed. Specifically, the human FSHR stable cells were dissolved in MACS buffer (PBS containing 0.5% BSA and 2 mM EDTA) at a concentration of 1×10⁶ cells/ml, and then added in an amount of 100 μl (1.0×10⁵ cells/test) to a 1.7 ml tube. The cells were then centrifuged at 500×g at 4° C. for 5 minutes. After removing the supernatant from the 1.7 ml tube, 100 μl each of the SAFA-FSH protein and the SL335 fragment that were diluted with MACS buffer at a concentration from 1,000 nM to 0.064 nM was added to the same tube to proceed a reaction at 4° C. for 30 minutes. After 500 μl of MACS buffer was added to the tube and the cells were centrifuged at 500×g at 4° C. for 5 minutes, the supernatant was removed therefrom to remove unbound proteins. 100 μl of goat anti-human IgG Fd-FITC (Southern Biotech, USA) antibody diluted with MACS buffer at a ratio of 1:500 was added to the tube to proceed a reaction at 4° C. for 30 minutes. After 500 μl of MACS buffer was added to the tube and the cells were centrifuged at 500×g at 4° C. for 5 minutes, the supernatant was removed therefrom to remove unbound antibodies. 200 μl of 0.4% PFA was added to the tube, and then the cells were loosened and fixed by using a pipette. By measuring the FITC sensitivity with a CytoFLEX device, the binding between the SAFA-FSH protein and the human FSHR was confirmed.

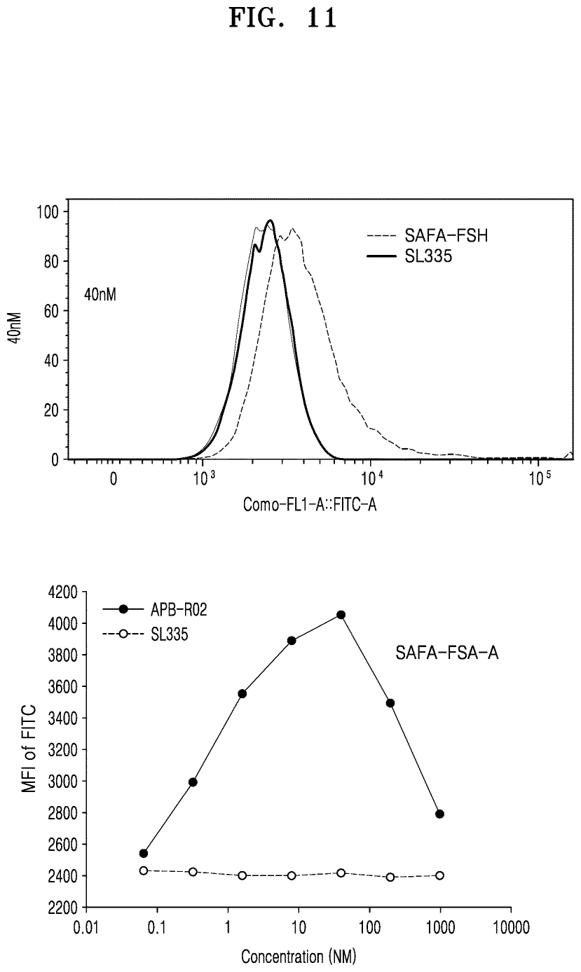

As a result, as shown in FIG. 11, the SL335 fragment did not form an effective bond with the human FSH receptors present on the surface of the FSHR stable cells, whereas the SAFA-FSH protein could form an effective bond with the human FSH receptors.

3-3. Confirmation of cAMP Regulatory Ability

By using the KGN cell line, which is a human ovarian granulosa cell line, the ability of the SAFA-FSH protein to regulate cAMP was confirmed. Specifically, 1 mL of the KGN cells was cultured overnight at a concentration of 3.0×10⁵ viable cells/well/mL in MEM+10 FBS medium contained in a 12-well culture plate. After allowing a reaction of 0.5 mM 3-isobutyl-1-methylxanthine with the cultured cells for 15 minutes, 0.5 mM IBMX was added thereto and cultured for 1 hour with the SAFA-FSH protein at a concentration of 10 ng/ml or 303 pM, Gonal-F, or FSH IS in an MEM medium supplemented with 1% FBS (NIBSC 08/282). A cAMP enzyme immunoassay kit (R&D, USA) was used to measure cAMP in the supernatant obtained after centrifugation of the cells at 500×g for 5 minutes at 4° C.

TABLE 4

| | cAMP (pM/well) | | | |
|---|---|---|---|---|
| | 10 ng/mL | Ratio % | 303 pM | Ratio % |
| FSH I.S. | 1740.935 | 100 | 1740.935 | 100 |
| rhFSH (Gonal-F) | 2021.169 | 116 | 2021.169 | 116 |
| SAFA-FSH | 1024.687 | 58.8 | 2452.84 | 141 |

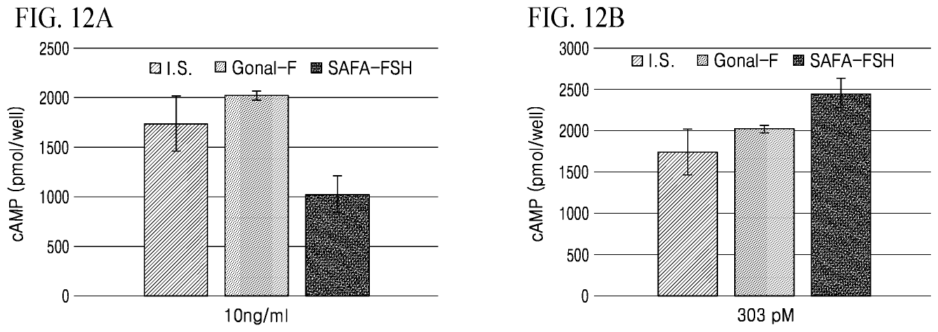

As a result, as shown in Table 4 and FIG. 12, it was confirmed that the SAFA-FSH protein could contribute to regulating the level of cAMP by acting on the human ovarian granulosa cell line.

### Example 4. Pharmacokinetic Evaluation of SAFA-FSH Protein

Absorption, distribution, in vivo changes, and excretion of the recombinant fusion protein according to an embodiment as prepared in Example 1 were confirmed through pharmacokinetics evaluation. Specifically, 17 healthy 7-week-old male rats (Specific pathogen free (SPF) rats, Hsd: Sprague DawleyR™ SDR™) were purchased from Koatech (South Korea) and acclimatized for 7 days, and healthy individuals among these rats were used in the present experiment. In each experimental group, 5 rats were administered intravenously with a single dose of the SAFA-FSH protein of Example 1 and Gonal-F protein (Follitropin alfa, rhFSH). The SAFA-FSH protein was administered at a dose of 200 μg/kg or 600 μg/kg, and the Gonal-F protein was administered at a dose of 88 μg/kg.

Afterwards, 0.5 ml of whole blood was collected through the jugular vein according to the prescribed blood sampling schedule [single intravenous administration of SAFA-FSH at doses of 200 μg/kg and 600 μg/kg, before administration of test substance (0) and after 5 minutes, 15 minutes, 30 minutes of administration of test substance, for 1, 1.5, 3, 5, 10, 24, 48, 72, 120, 144, and 168 hours (total of 15 points); single intravenous administration of Gonal-F at a dose of 88 μg/kg, before administration of test substance (0) and after 5 minutes, 15 minutes, and 30 minutes of administration of test substance, for 1, 1.5, 3, 5, 10, 24, 48, and 72 hours (total of 12 points)]. Serum was separated by centrifugation at 13,000 rpm for 2 minutes and stored in an ultra-low temperature freezer (−70° C.). Next, the protein concentration in plasma was quantitatively analyzed by ELISA method.

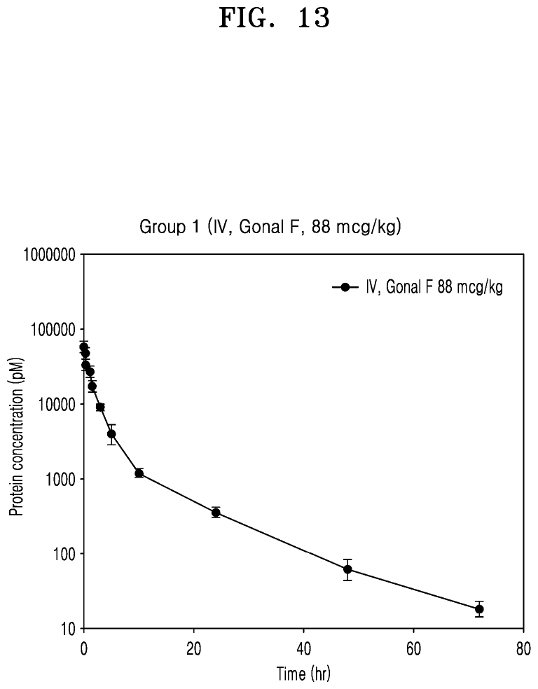

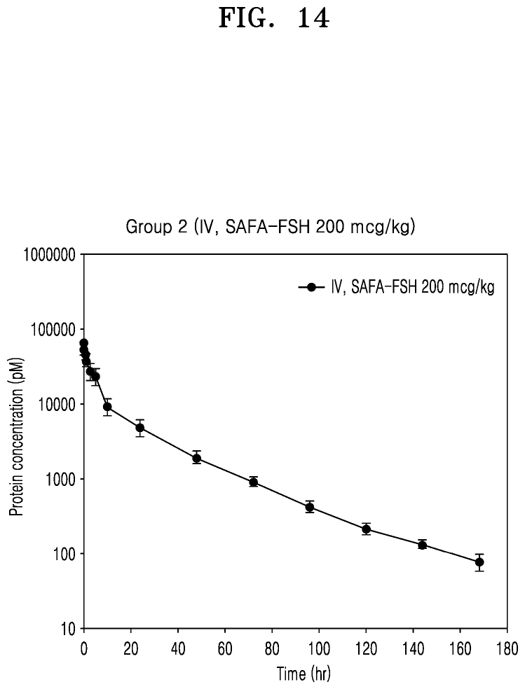

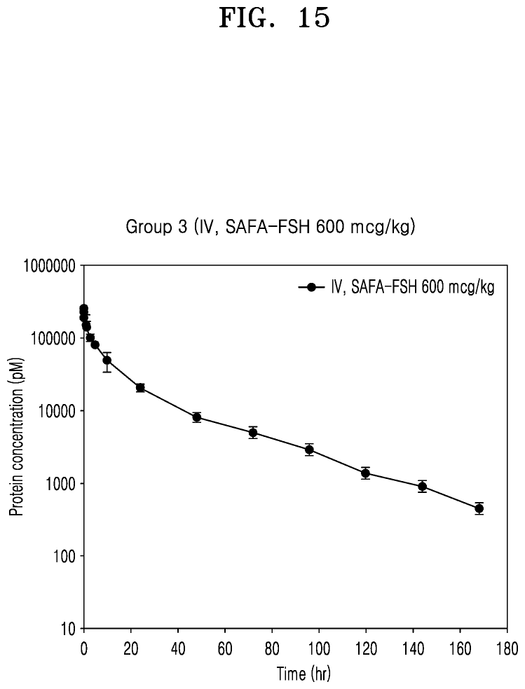

As a result, as shown in FIG. 13 to 15, it was confirmed that the highest serum concentration (Cmax) of the SAFA-FSH protein was similar to or about 1.2 times higher than that of the Gonal-F protein. In addition, the elimination half-life of the SAFA-FSH protein was 27.1 hours or 10.4 hours, which was 2.6 times longer than that of the Gonal-F protein. Referring to the experimental results above, it can be seen that the recombinant fusion protein has increased elimination half-life in the body and can provide convenience to patients due to a long drug administration interval.

### Example 5. Pharmacodynamics Evaluation of SAFA-FSH Protein

The pharmacological effect of the recombinant fusion protein according to an embodiment as prepared in Example 1 on the treatment of female infertility was confirmed through pharmacodynamics evaluation. Specifically, by using 20 2-week-old immature female Sprague Dawley rats (30 g to 40 g), the SAFA-FSH protein of Example 1 and the Gonal-F protein were subcutaneously administered to the rats under the experimental conditions shown in Table 5, so as to confirm the growth of ovaries stimulating follicles. From the time of administration of the test substances to Day 7, the ovaries were removed from the rats and weighed, so as to confirm the growth of ovaries by the SAFA-FSH protein.

TABLE 5

| | Administration | Administration | Dosing |
|---|---|---|---|
| Sample | group | condition | interval |
| SAFA- | SAFA-FSH 800 | 20 mcg/ml (~800 ng/rat) + | 0 d, 3 d |
| FSH | ng/rat E3D | 14 IU hCG | |
| | SAFA-FSH 2.4 | 60 mcg/ml (~2.4 mcg/rat) + | 0 d, 3 d |
| | ng/rat E3D | 14 IU hCG | |
| Gonal-F | hFSH 240/rat ED | 6 mcg/kg (~240 ng/rat) + | 0 d, 1 d, 2 d, |
| | | 14 IU hCG | 3 d, 4 d, 5 d |
| | hFSH 240/rat E3D | 6 mcg/kg (~240 ng/rat) + | 0 d, 3 d |
| | | 14 IU hCG | |
| Vehicle | Vehicle | Buffer + 14 IU hCG | 0 d, 1 d, 2 d, |
| | | | 3 d, 4 d, 5 d |

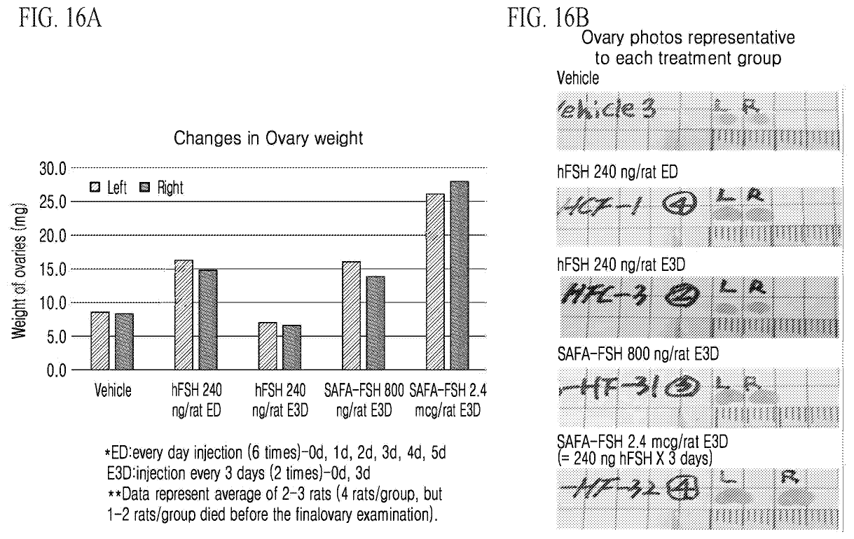

As a result, as shown in FIG. 16, the hFSH 240/rat ED group among the Gonal-F protein-administered groups induced effective growth of the ovaries compared to the control group, whereas the hFSH 240/rat E3D group with a long administration interval did not affect the growth of ovaries. On the other hand, the SAFA-FSH protein administration group according to the present example induced effective growth of the ovaries despite a long administration interval of 3 days. In particular, the growth of the ovaries was induced similar to or about 3 times higher than the hFSH 240/rat ED group.

In addition, to confirm the efficacy of the SAFA-FSH protein, pharmacodynamics experiments were performed by using an experimental animal model having hypogonadism prepared by administering Diphereline PR injection to male rats (Sprague Dawley). Specifically, 5-week-old male rats were divided into 7 groups of 5 or 6 rats per group. Weight gain was measured by recording the weight of the experimental animals at 7-day intervals. At the start of the experiment and at 3 weeks after the experiment, Diphereline PR injection (Ipsen, Paris, France) was subcutaneously administered at a dose of 5 mg/kg was to all experimental groups except Group 1. To all experimental groups, except for Group 1 and Group 2, human chorionic gonadotropin (hCG) protein (LG Chem, South Korea) was subcutaneously administered at a dose of 20 IU/kg three times per week for 9 weeks, and the recombinant FSH protein was subcutaneously administered at a dose of 1.85 μg/kg (25 IU/kg) three times per week (Group 4) or once every 5 days (Group 5). The SAFA-FSH protein was subcutaneously administered at a dose of 9.87 μg/kg once every 5 days (Group 6) or once every 10 days (Group 7). To measure testosterone levels in each experimental group, rats were anesthetized and blood was collected at regular times every week. The collected blood was centrifuged at 3,000 rpm for 10 minutes to separate serum, and testosterone levels were measured by using the electrochemiluminescence method at Seoul Clinical Laboratories, South Korea. At Week 9 elapsed from the time of administration of the test substance, the rats were sacrificed. To measure the number of sperms of the sacrificed rats, the epididymis cauda was placed on a 6 cm plate with 10 mL DPBS, minced with a scalpel, and then reacted in saline under conditions of a temperature of 37° C. and 5% CO₂ for 15 minutes. Then, heat treatment was performed thereon at 60° C. for 1 minute, immediately followed by cooling at 24°. The appropriately diluted samples were used to count the number of sperms by using a Neubauer hemocytometer.

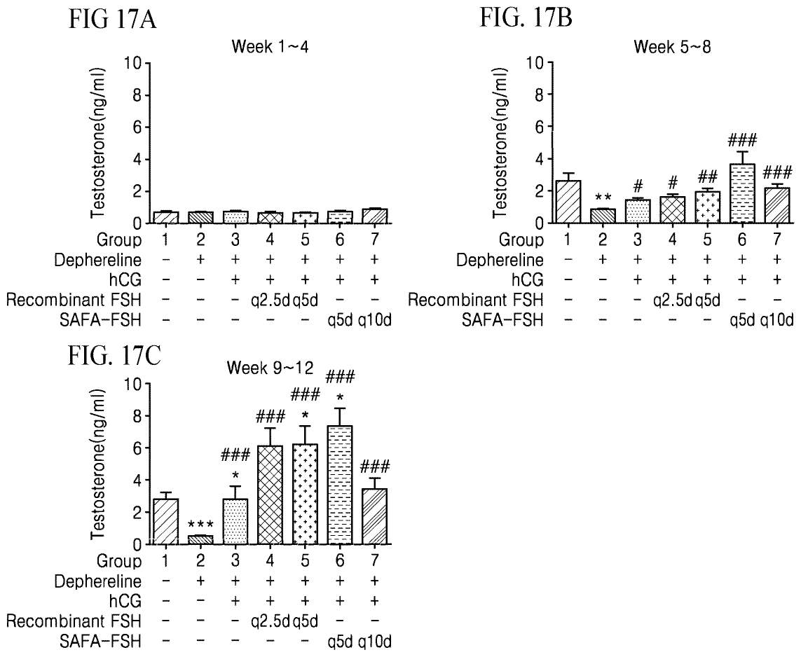

As a result, no difference in body weight was observed between the groups by the test substance during the experiment period. In addition, as shown in FIG. 17, the average serum testosterone level for 1 week to 4 weeks after administration of the test substance did not show any difference in all test groups (FIG. 17A). On the other hand, in Group 1 not administered with Diphereline injection, the average serum testosterone level increased for 5 weeks to 8 weeks after administration of the test substance, whereas in Group 2 administered with Diphereline injection, the average serum testosterone level was measured lower than Group 1. Groups 3, 4, 5, 6, and 7 treated with hCG showed an increase in the average serum testosterone levels compared to Group 2 not administered with hCG (FIG. 17B). In addition, for 9 weeks to 12 weeks after administration of the test substance, the average serum testosterone level of Group 2 was maintained at a lower level compared to that of Group 1. Groups 3, 4, 5, 6, and 7 showed an increase in the average serum testosterone level compared to Group 2. In particular, the average serum testosterone levels of Groups 5 and 6 were significantly higher than those of Group 1 (FIG. 17C).

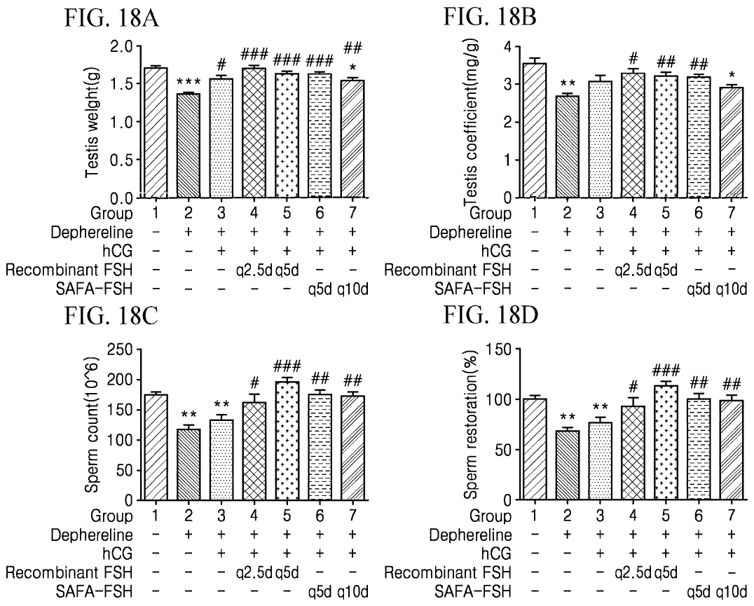

In addition, at Week 9 elapsed from the time of administration of hCG and the recombinant FSH (rFSH), the rats were sacrificed and testes were weighed. The testis weight in Group 2 was lower than Group 1, whereas the testis weight in Groups 3, 4, 5, 6, and 7 was measured higher than in Group 2 (FIG. 18A). In addition, the testicular coefficient value (i.e., a ratio of testis/body weight) showed similar results to the testis weight (FIG. 18B). The total number of sperms in Group 2 decreased by 68% compared to Group 1, and the number of sperms in Group 3 did not recover compared to Group 2. In addition, it was confirmed that the number of sperms was recovered in Group 4 administered with the rFSH three times every 7 days and Group 5 administered with the rFSH once every 5 days, and that the number of sperms was recovered in Group 6 administered with the SAFA-FSH once every 5 days and Group 7 administered with the SAFA-FSH once every 10 days (FIGS. 18C and 18D).

Referring to the results above, it can be seen that the SAFA-FSH (recombinant fusion protein) can increase the drug administration interval and achieve high therapeutic efficacy.

According to the one or more embodiments, a recombinant fusion protein including: an antigen-binding fragment that binds to serum albumin; and human follicle stimulating hormone has improved pharmacokinetic properties including increased in vivo half-life and can continuously exhibit therapeutic efficacy such as ovarian growth promotion.

Therefore, the recombinant fusion protein can be used as an active ingredient in a human follicle stimulating hormone formulation for treatment of infertility.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details can be made therein without departing from the spirit and scope of the disclosure as defined by the following claims.

All of the various aspects, embodiments, and options described herein can be combined in any and all variations. All publications, patents, and patent applications mentioned in this specification are herein incorporated by reference to the same extent as if each individual publication, patent, or patent application was specifically and individually indicated to be herein incorporated by reference.

What is claimed is:

1. A recombinant fusion protein comprising (a) a human follicle stimulating hormone (hFSH) comprising an hFSH alpha subunit and an hFSH beta subunit and (b) an antigen binding fragment (Fab) that binds to serum albumin.

2. The recombinant fusion protein of claim 1, wherein the hFSH alpha subunit and the hFSH beta subunit are linked to terminal regions of the Fab.

3. The recombinant fusion protein of claim 1 or 2, wherein the hFSH alpha subunit and the hFSH beta subunit are independently linked to a terminus of the heavy chain constant 1 domain and a terminus of the light chain constant domain of the Fab.

4. The recombinant fusion protein of any one of claims 1-3, wherein the hFSH alpha subunit and the hFSH beta subunit are independently linked by a linker to a terminus of a heavy chain constant 1 domain and a terminus of a light chain constant domain of the Fab.

5. The recombinant fusion protein of any one of claims 1-4, wherein the hFSH alpha subunit is linked to a terminus of the light chain constant domain and the hFSH beta subunit is linked to a terminus of the heavy chain constant 1 domain of the Fab.

6. The recombinant fusion protein of claim 4 or 5, wherein the linker comprises 1 to 50 amino acids.

7. The recombinant fusion protein of claim 6, wherein the linker comprises an amino acid sequence of any one of SEQ ID NOS:16, 70-85, and 88.

8. The recombinant fusion protein of any one of claims 1-7, wherein the hFSH alpha subunit comprises an amino acid sequence having at least 90% identity to SEQ ID NO:1.

9. The recombinant fusion protein of any one of claims 1-8, wherein the hFSH alpha subunit comprises an amino acid sequence of SEQ ID NO:1.

10. The recombinant fusion protein of any one of claims 1-9, wherein the hFSH beta subunit comprises an amino acid sequence having at least 90% identity to SEQ ID NO:4.

11. The recombinant fusion protein of any one of claims 1-10, wherein the hFSH beta subunit comprises an amino acid sequence of SEQ ID NO:4.

12. The recombinant fusion protein of any one of claims 1-11, wherein the Fab comprises
a heavy chain comprising a heavy chain variable domain comprising
(a) a heavy chain complementarity determining domain 1 (CDR1) comprising the amino acid sequence of SYGIS (SEQ ID NO:22),
a heavy chain complementarity determining domain 2 (CDR2) comprising the amino acid sequence of WINTYSGGTKYAQKFQG (SEQ ID NO:23), and
a heavy chain complementarity determining domain 3 (CDR3) comprising the amino acid sequence of LGHCQRGICSDALDT (SEQ ID NO:24);
(b) a heavy chain CDR1 comprising the amino acid sequence of SYGIS (SEQ ID NO:22),
a heavy chain CDR2 comprising the amino acid sequence of RINTYNGNTGYAQRLQG (SEQ ID NO:25), and
a heavy chain CDR3 comprising the amino acid sequence of LGHCQRGICSDALDT (SEQ ID NO:24);
(c) a heavy chain CDR1 comprising the amino acid sequence of NYGIH (SEQ ID NO:26),
a heavy chain CDR2 comprising the amino acid sequence of SISYDGSNKYYADSVKG (SEQ ID NO:27), and
a heavy chain CDR3 comprising the amino acid sequence of DVHYYGSGSYYNAFDI (SEQ ID NO:28);
(d) a heavy chain CDR1 comprising the amino acid sequence of SYAMS (SEQ ID NO:29),
a heavy chain CDR2 comprising the amino acid sequence of VISHDGGFQYYADSVKG (SEQ ID NO:30), and
a heavy chain CDR3 comprising the amino acid sequence of AGWLRQYGMDV (SEQ ID NO:31);
(e) a heavy chain CDR1 comprising the amino acid sequence of AYWIA (SEQ ID NO:32),
a heavy chain CDR2 comprising the amino acid sequence of MIWPPDADARYSPSFQG (SEQ ID NO:33), and
a heavy chain CDR3 comprising the amino acid sequence of LYSGSYSP (SEQ ID NO:34); or
(f) a heavy chain CDR1 comprising the amino acid sequence of AYSMN (SEQ ID NO:35),
a heavy chain CDR2 comprising the amino acid sequence of SISSSGRYIHYADSVKG (SEQ ID NO:36), and
a heavy chain CDR3 comprising the amino acid sequence of ETVMAGKALDY (SEQ ID NO:37); and
a light chain comprising a light chain variable domain comprising
(g) a light chain CDR1 comprising the amino acid sequence of RASQSISRYLN (SEQ ID NO:38),
a light chain CDR2 comprising the amino acid sequence of GASRLES (SEQ ID NO:39), and
a light chain CDR3 comprising the amino acid sequence of QQSDSVPVT (SEQ ID NO:40);
(h) a light chain CDR1 comprising the amino acid sequence of RASQSISSYLN (SEQ ID NO:41),
a light chain CDR2 comprising the amino acid sequence of AASSLQS (SEQ ID NO:42), and
a light chain CDR3 comprising the amino acid sequence of QQSYSTPPYT (SEQ ID NO:43);
(i) a light chain CDR1 comprising the amino acid sequence of RASQSIFNYVA (SEQ ID NO:44),
a light chain CDR2 comprising the amino acid sequence of DASNRAT (SEQ ID NO:45), and
a light chain CDR3 comprising the amino acid sequence of QQRSKWPPTWT (SEQ ID NO:46);
(j) a light chain CDR1 comprising the amino acid sequence of RASETVSSRQLA (SEQ ID NO:47),
a light chain CDR2 comprising the amino acid sequence of GASSRAT (SEQ ID NO:48), and
a light chain CDR3 comprising the amino acid sequence of QQYGSSPRT (SEQ ID NO:49);
(k) a light chain CDR1 comprising the amino acid sequence of RASQSVSSSSLA (SEQ ID NO:50),
a light chain CDR2 comprising the amino acid sequence of GASSRAT (SEQ ID NO:48), and
a light chain CDR3 comprising the amino acid sequence of QKYSSYPLT (SEQ ID NO:51); or
(1) a light chain CDR1 comprising the amino acid sequence of RASQSVGSNLA (SEQ ID NO:52),
a light chain CDR2 comprising the amino acid sequence of GASTGAT (SEQ ID NO:53), and
a light chain CDR3 comprising the amino acid sequence of QQYYSFLAKT (SEQ ID NO:54).

13. The recombinant fusion protein of any one of claims 1-12,
wherein the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and
wherein the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

14. The recombinant fusion protein of any one of claims 1-13, wherein the heavy chain variable domain comprises an amino acid sequence having at least 90% identity to SEQ ID NO:55, 56, 57, 58, 59, or 60.

15. The recombinant fusion protein of any one of claims 1-14, wherein the heavy chain variable domain comprises the amino acid sequence of SEQ ID NO:60.

16. The recombinant fusion protein of any one of claims 1-15, wherein the light chain variable domain comprises an amino acid sequence having at least 90% identity to SEQ ID NO:61, 62, 63, 64, 65, 66, or 67.

17. The recombinant fusion protein of any one of claims 1-16, wherein the light chain variable domain comprises the amino acid sequence of SEQ ID NO:67.

18. The recombinant fusion protein of any one of claims 1-17, wherein the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67.

19. The recombinant fusion protein of any one of claims 1-18, wherein the heavy chain constant domain comprises an amino acid sequence having at least 90% identity to SEQ ID NO:68.

20. The recombinant fusion protein of any one of claims 1-19, wherein the light chain constant domain comprises an amino acid sequence having at least 90% identity to SEQ ID NO:69 or 91.

21. The recombinant fusion protein of any one of claims 1-20, wherein the Fab comprises a heavy chain comprising the amino acid sequence of SEQ ID NO:6 and a light chain comprising the amino acid sequence of SEQ ID NO:91.

22. The recombinant fusion protein of any one of claims 1-21, wherein the Fab comprises a heavy chain constant 1 domain comprising the amino acid sequence of SEQ ID NO:68 linked to the heavy chain variable domain; and a light chain constant domain comprising the amino acid sequence of SEQ ID NO:69 or 91 linked to the light chain variable domain.

23. A nucleic acid encoding the recombinant fusion protein of any one of claims 1-22.

24. An expression vector comprising the nucleic acid of claim 23.

25. A cell transformed with the expression vector of claim 24.

26. A composition comprising the recombinant fusion protein of any one of claims 1-22 and a carrier.

27. A pharmaceutical composition comprising the recombinant fusion protein of any one of claims 1-22 and a pharmaceutically acceptable carrier.

28. A kit comprising the composition of claim 26 or 27 and a label comprising instructions for a use.

29. A method of inducing superovulation of a subject in need thereof, comprising applying the pharmaceutical composition of claim 27 to in vitro fertilization-embryo transfer (IVF-ET), gamete intrafallopian transfer (GIFT), zygote intrafallopian transfer (ZIFT), intracytoplasmic sperm injection (ICSI), or in vitro fertilization.

30. A method of treating anovulation, hypogonadism, or polycystic ovary syndrome in a subject in need thereof, comprising administering the pharmaceutical composition of claim 27 to the subject.

31. The method of claim 29 or 30, wherein the composition is administered subcutaneously or intramuscularly to the subject.

32. A long-acting human follicle stimulating hormone (hFSH) composition comprising the recombinant fusion protein of any one of claims 1-22 as an active ingredient.

33. The long-acting hFSH formulation of claim 32, wherein the long-acting hFSH formulation is applied to in vitro fertilization-embryo transfer (IVF-ET), gamete intrafallopian transfer (GIFT), zygote intrafallopian transfer (ZIFT), intracytoplasmic sperm injection (ICSI), or in vitro fertilization, to induce superovulation of a subject.

34. The long-acting hFSH formulation of claim 33, wherein the long-acting hFSH formulation is used for treatment of anovulation, hypogonadism, or polycystic ovary syndrome.

35. The long-acting hFSH formulation of claim 33, wherein the long-acting hFSH formulation is administered subcutaneously or intramuscularly.
